(19) United States
(12) Patent Application Publication
(10) Pub. No.: US 2002/0102674 A1
(43) Pub. Date: Aug. 1, 2002

(54) STABILIZED MICROPOROUS MATERIALS

(76) Inventor: DAVID M ANDERSON, AMHERST, NY (US)

Correspondence Address: David M. McConoughey, Stoll, Miskin, Hoffman and Badie, 350 Fifth Avenue, Suite 6110, New York, NY, 10118, US

(21) Appl. No.: 08/272,334

(22) Filed: Jul. 7, 1994

Related U.S. Application Data
(63) Continuation-in-part of application No. 08/156,386, filed on Nov. 22, 1993, now abandoned.
Application No. 08/156,386 is a continuation of application No. 08/058,045, filed on May 4, 1993, now abandoned.
Application No. 08/058,045 is a continuation of application No. 07/809,231, filed on Dec. 17, 1991, now abandoned.
Application No. 07/809,231 is a continuation of application No. 07/564,695, filed on Aug. 7, 1990, now abandoned.
Application No. 07/564,695 is a continuation of application No. 07/292,615, filed on Dec. 30, 1988, now abandoned.
Application No. 07/292,615 is a continuation-in-part of application No. 07/052,713, filed on May 20, 1987, now abandoned.

(51) Int. Cl. C12N 11/00; C12N 11/16

(52) U.S. Cl. 435/174

(57) ABSTRACT

The invention involves a polymeric, microporous membrane material characterized by a continuous-triply-periodic, highly branched and interconnected pore space morphology having a globally uniform, pre-selected pore size, characterized by high porosity. And further involves several related methods for forming mircorporous membrane materials; including polymerization of the hydrophobic component in a ternary surfactant/water/hydrophobe cubic phase, and other thermodynamically stable or metastable phases of phase-segregated systems, especially systems which are substantially ternary or binary, and particualarly directed to applications of the novel material in: immobilization, encapsulation, and/or controlled release of biologically active agents, and other applications where a controlled pore size is necessary or advantageous.

### REFERENCE TO RELATED APPLICATION

This application is a continuation-in-part of application Ser. No. 08/156,386, filed Nov. 22, 1993 which is a continuation of Ser. No. 08/058,045 filed May 4, 1993 now abandoned, which was a continuation of Ser. No. 07/809,231 filed Dec. 17, 1991, now abandoned, which was a continuation of Ser. No. 07/564,695 filed Aug. 7, 1990, now abandoned, which was a continuation of Ser. No. 07/292,615 filed Dec. 30, 1988, now abandoned, which was a continuation-in-part of Ser. No. 07/052,713 filed May 20, 1987.

### FIELD OF THE INVENTION

The present invention is in the field of microporous membrane materials, especially polymeric membranes,and particularly the use of such materials in connection with biologically active agents, in critical filtrations, and in applications involving microstructure such as critical phase transition measurements, microelectronics etc.

The past 20 years has seen tremendous growth in the applications of polymeric membranes, not only in filtration—microfiltration (MF), ultrafiltration (UF), and hyperfiltration or reverse osmosis (RO)—but also in a variety of other areas such as fuel cells and batteries, controlled-release devices as for drug or herbicide metering, dialysis and electrodialysis, pervaporation, electrophoresis, membrane reactors, ion-selective electrodes, and as supports for liquid membranes, to name some important areas. Furthermore, modification of neutral polymer membranes can yield ionomeric or ‘ion-exchange’ membranes which are finding increasing application in many chemical, electrochemical, filtration and even biochemical processes. In many applications the availability of a membrane with precisely-controlled porespace and high porosity would represent a significant technological advance.

### BACKGROUND ART

The ultimate membrane would have identical, highly interconnected pores comprising a porespace with perfect three-dimensional periodic order. This ideal has been approached in the development of polymeric microporous membranes but never achieved. The simplest type of sieve is a net filter, where each layer in the filter is a woven mesh. The geometry of the pore space in a given layer is thus a close approximation to a finite portion of a doubly-periodic net, the latter being a mathematical idealization with perfect regularity within the plane. Note that if, in addition, these double-periodic layers are stacked at regular intervals with all layers in vertical registry, the resulting sieve is triply-periodic. Woven mesh filters are not available with pore sizes less than about 60 microns, so they cannot be used for reverse osmosis, ultrafiltration, nor even microfiltration.

Another doubly-periodic geometry that is achieved in some filters is that of hexagonally close-packed cylindrical pores. For example, glass capillary bundle filters are made from close-packed arrays of parallel glass capillaries. Capillary arrays can also be formed from hollow fibers of organic polymers, although these are not yet available commercially. A major drawback of cylindrical-pore filters is the lack of porespace branchings and reconnections, which leaves only one pathway for a fluid particle entering a given pore; thus clogging becomes a serious problem, as does sensitivity to handling. Of course, cylindrical pores can provide a narrow distribution of pore sizes without necessarily lying on a doubly-periodic lattice; for example, nucleation-track filters have randomly placed parallel cylindrical pores. But this randomness means that the number of pores per unit cross-sectional area must be kept small to maintain monodispersity, so that these filters have the additional drawback of low porosity and thus low filtration rates. Nevertheless, nucleation-track filters are considered the best membrane filters available for sieving below 60 microns, despite these obvious drawbacks.

U.S. Pat. No. 4,280,909 describes a microporous membrane which is, strictly speaking, triply-periodic, but the topology of the porespace is exactly the same as in the capillary array membranes, namely the flow channels are strictly linear and there are no porespace branchings or reconnections. The periodicity in the third dimension refers only to the vertical stacking of tapered pores of equal height, so that the cylindrical pores of the capillary array membrane have become instead tubular pores with a periodically varying diameter. This membrane does not satisfy one of the most important desired features, namely the intricate yet controlled porespace. A precisely defined porespace with branchings and reconnections, in which each identical pore body connects to exactly the same number of other pore bodies through identical pore throats, is important in:

a) reducing clogging, as when the membrane is used for filtration, for example;

b) enhancing mixing, as when the membrane is used in catalysis or ion exchange, for example; and

c) providing accessible channels and pore bodies of specific shape, as when the membrane is used in the preparation of metal microstructures (Jacobs et al., Proceedings of Bremen Workshop of Sep. 22-24, 1982, Elsevier Scientific Pub. Co., Amsterdam), for example.

Sintered-particle membranes have intricate three-dimensional porespace with many interconnections, but have oddly-shaped and polydisperse pores as well as low pore density, the latter drawback being the primary reason they have been generally replaced by membrane filters. Most sintered-particle filters have retention ratings at or above 0.7 microns.

The membrane that is most commonly used in particle filtration has high porosity but a random, irregular porespace that makes it generally unusable as a sieve. Distributions of pore radii in cellulose nitrate membrane filters have been measured using mercury porisimetry, and the distributions are very broad: the full-width at half-maximum (FWHM) of the distribution is about equal to the average radius (Brock, T. D., Membrane Filtration: A User's Guide and Reference Manual, Science Tech, Inc., Madison Wis., page 57 (1983)).

In the realm of nonpolymeric sieves, zeolites provide fairly well-controlled, triply-periodic pore networks, but the free diameters of apertures governing access to channels are generally less than 2 nm, and in fact nearly always less than 1 mm (Barrer, R. M., Zeolites and Clay Minerals As Sorbents And Molecular Sieves, Academic Press, London, (1978)); also the porosities of zeolites (defined as cc's of water per cc of crystal) are nearly always less than 50%. Furthermore, most zeolites selectively absorb polar molecules because most are themselves highly polar, having high local electrostatic fields and field gradients (Barrer, R. M., Zeolites and Clay Minerals As Sorbents And Molecular Sieves, Academic Press, London, (1978)). Perhaps most importantly, the macroscopic size of zeolite crystals has very serious practical limitations making such materials unsuitable for forming reasonably large membrane-like structures with the necessary degree of continuity.

These and other difficulties with prior materials and methods have obviated in a novel and inventive manner by the present invention.

### SUMMARY OF THE INVENTION

The invention involves a polymeric, microporous membrane material characterized by a continuous, triply-periodic, highly branched and interconnected pore space morphology having a globally uniform, pre-selected pore size. The pore size ranges from two nanometers to sixty microns, preferably in the range off two nanometers to one micron and particularly preferably on the order of ten nanometers. The material of the invention is characterized by high porosity: greater than fifty percent and, for certain applications, greater than ninety percent. The invention involves controlled variation of the pore characteristics, particularly the electro-chemical characteristics.

The invention involves several related methods for forming microporous membrane materials, including polymerization of the hydrophobic component in a ternary surfactant/water/hydrophobe cubic phase, and other thermodynamically stable or metastable phases of phase-segregated systems, especially systems which are substantially ternary or binary.

In one aspect the invention is particularly directed to materials developed from an equilibrium cubic phase of a binary or ternary system (hydrophobic/hydrophilic/surfactant) in which any of the oil, aqueous, or surfactant phases is polymerized after equilibration.

A further aspect of the invention is particularly directed to applications of these novel materials in: immobilization, encapsulization, and/or controlled release of biologically active agents such as enzymes, other proteins, cell fragments, and intact cells, especially making use of biocompatible materials; critical filtrations including chiral separations, affinity-based separations, dialysis, protein sieving, and active transport; processes such as measure of critical phase transitions; and in microelectronics, molecular electronics, and bio-electronics; and other applications where a controlled pore space is necessary or advantageous.

### DESCRIPTION OF THE DRAWINGS

FIG. 1 shows small-angle x-ray scattering data from membrane material according to the present invention. Individual marks represent recorded intensities at each channel. Vertical lines indicate theoretical peak positions for a structure of space group Im3m and lattice parameter 11.8 nm. the label on the abscissa is s=2 sin (theta)/lambda, where theta is one-half the scattering angle and lambda is the wave length off the radiation used. The large peak at s=0.0025/Angstrom is due to the main beam, and is not a reflection.

FIG. 2 shows an electron micrograph of membrane material according to the invention. Dark regions correspond to PMMA, and light regions to void. Regions of particularly good order are outlined. (Magn. 1,000,000).

FIG. 3 is the optical diffraction pattern of the negative used to make FIG. 2. The eight-spot pattern indicated with circles provides further demonstration of cubic symmetry.

FIG. 4, A, B and C are computer-generated pictures of a theoretical model structure, from Anderson, 1986, the applicant's doctoral thesis. The surface has constant mean curvature, and divides space into two interpenetrating labyrinths, one threaded by graph A and the other by graph B.

A) (upper). Computer graphic, viewed approximately along the (110) direction.

B) Projection in the (111) direction.

C) (lower). Line drawing, without hidden line removal, from an oblique angle.

FIG. 5A and B show digitized electron micrograph of:

A) a bicontinuous cubic phase in a star-block PI/PS copolymer, and

B) a prediction using a bicontinuous model from the applicant's doctoral thesis, Anderson, 1986.

The model used was determined by the constant-mean-curvature surface of the ‘D’ family (Pn3m symmetry) which matches the volume fractions of the sample. A computer was used to send projection rays through the theoretical model, and the grey level at each pixel calculated.

FIG. 6 combines the views of FIGS. 5 A and B for clearer comparison.

FIG. 7 sets out three equations used in the calculation of the behavior of block copolymers.

FIG. 8 illustrates some results from evaluation of size and dispersity of pore sizes in certain cubic phases by thermoporimetry.

FIG. 9 illustrates some results from evaluation of size and dispersity of pore sizes in certain cubic phases by thermoporimetry.

FIG. 10 A and B are computer graphics illustrating the microstructure of the present invention.

FIG. 11 is a ternary phase diagram of the system {didodecyldimethylammonium bromide/water/hexene} at 25° C.

FIG. 12 is a computer graphic of the I-WP minimal surface.

FIG. 13 A, B, and C are computer line drawing off three representatives of the I-WP family of constant mean curvature surfaces.

FIG. 14 shows small-angle X-ray scattering data from a polymerized cubic phase.

FIG. 15 is a transmission electron micrograph of a polymerized cubic phase.

FIG. 15B is a computer simulation of the micrograph of FIG. 15A.

FIG. 16 is a split-screen image, with a TEM micrograph of a cubic structure in a (polystyrene/polyisoprene) star diblock copolymer on the left half, and a computer simulation using structure indicated in FIG. 10 on the right half.

FIGS. 17-19 are phase Diagrams showing the location of the L3 phase.

FIG. 20 is a mathematical idealization of the surfactant bilayer in cross-section.

FIG. 21, A, B, C and D, FIG. 22, FIG. 23 A and B and FIG. 24 are phase diagrams showing the location of the L3 phase in various systems.

FIG. 25 shows schematically the relation between the areas A_{EO} and A_{HC}, drawn as spherical caps, and the spontaneous radius curvature R_{O}=1/H_{O}.

### CLARIFICATION OF SOME TECHNICAL TERMS.

Membrane. This word has two quite distinct meanings, but fortunately these can easily be distinguished from the context. One meaning relates to a microporous material, generally fabricated to be of very small thickness, but much larger in the other two dimensions. The other meaning is much more microscopic, and originates from biological contexts. This second meaning is that of a lipid bilayer (into which are incorporated enzymes), which serves to separate different regions of the cell, or to enclose the cell itself, or more generally it refers too the generic bilayer independently of any biological function it may serve (such as used by theoreticians who study surfactant bilayers and their properties).

Mean curvature, Gaussian curvature. At each point on a smooth surface, there are two directions along which the normal curvature is greatest and least. The values of these curvatures (which are reciprocals of radii of curvature) are called the principle curvatures. One-half the sum of these curvatures is called the mean curvature, and the product of these curvatures is the Gaussian curvature. In bicontinuous cubic phases, at most points on the midplane surface the surface is saddle-like, with principle curvatures in opposite directions, so that the Gaussian curvature is negative and the mean curvatures is generally small in magnitude (due to a partial cancellation when summing the two curvatures).

Minimal surface, constant mean curvature surface, spontaneous mean curvature. A surface which has zero mean curvature at each point is called a minimal surface, by definition. A surface which has the same value of mean curvature at each point on the surface is called a surface of constant mean curvature (or an ‘H-surface’ for short). H-surfaces are important for two reasons: first of all, they minimize surface area under a volume fraction constrain; second, and more importantly here, the balance of stearic, van der Waals, and electrostatic forces between surfactant molecules (and other molecules which may penetrate into the surfactant film) determines a “preferred” or “spontaneous” mean curvature of the film, which in most interpretations is registered at the polar/apolar interface at or just inside of the surface describing the location of the surfactant head groups; since the composition of the surfactant film is rather homogeneous in most cases, a surface of constant mean curvature is a very good representation of the interface.

Bicontinuous. A material in which two or more components are continuous simultaneously. Most authors define continuous in terms of the existence of sample-spanning paths in all three directions. Thus, the lamellar phase is not bicontinuous, because there are no sample-spanning paths in a direction perpendicular to the lamellae. Some authors use a much stronger definition, namely that it is possible, for either component, to connect any two points lying in the same component (say, water) with a path through only that component. The bicontinuous cubic phases satisfy both definitions, so that this difference in definitions does not pose any difficulty. It should be noted that in a ternary surfactant/oil/water bicontinuous phase (e.g., a cubic phase, microemulsion, or L3 phase), the surfactant is also continuous by necessity, and thus the structure is actually tricontinuous; however, this latter term has not been adopted by the community.

Triply-periodic. Possessing periodicity in three directions, which are linearly independent; that is, none is simply a linear combination of the other two (thus, the third vector points outside of the plane determined by the first two). An infinitely wide checkerboard would be doubly-periodic; a lattice of gold atoms is triply-periodic (in the present context we do not require infinite extent).

Birefringent. Having different refractive indices in different directions. This property is, with transparent materials, very easy to test for, because birefringent materials placed between polarizing lenses oriented at right angles allow light to pass through, and usually give rise to beautiful colors and textures through such crossed polars. The lamellar and hexagonal phases are generally birefringent, because there is an orientation of carbon-carbon bonds of the hydrocarbon tails with respect to the optic axis (which is normal to the lamellae in the lamellar phase, and the long cylinders in the hexagonal phase). The (unstrained) cubic phases are non-birefringent by virtue of the equivalence of the principle directions.

Vesicle; Liposome. If a surfactant bilayer closes up to form a closed, often roughly-spherical, sack enclosing an aqueous interior and also having an aqueous exterior, then this is called a unilamellar vesicle (ULV). A nesting of such vesicles is called a multilamellar vesicle (MLV). By convention, when such structures are made from lipids they are called liposomes. Most liposomes have diameters measured in microns. Most are also rather dilute in surfactant, although under certain conditions the separation between the bilayers can become approximately the same as the bilayer thickness itself, so that the volume fraction of surfactant is on the order of one-half within the liposome, and in some such cases x-ray diffraction exhibits Bragg peaks indicating periodic order in the lamellar spacing.

Highly-connected. A surface which has a property, that any closed loop on the surface can be reduced to a point by continuously shrinking the loop without ever leaving the surface is called simply-connected. More complicated surfaces are not simply-connected, the simplest multiply-connected surface being a circular annulus; the annulus is in fact doubly-connected, because a single cut in the surface (such as a radial cut) can reduce the surface to a simply-connected one. The surface which describes the midplane of the bilayer in a surfactant/water bicontinuous cubic phase is very highly-connected, and in fact the unbounded, triply-periodic idealization of this surface is infinitely-connected.

### DETAILED DESCRIPTION OF THE INVENTION

A bicontinuous morphology is distinguished by two interpenetrating, labyrinthine networks of ordinarily immiscible substances (Scriven, L. E., Nature 263:123(1976)), in which macroscopic phase separation is prevented by one of at least two possible means: 1) chemical linking between the two components, as in block copolymers; or 2) addition of surfactant. A triply-periodic bicontinuous morphology (TPBM hereafter) is further distinguished by long-range three-dimensional periodic ordering conforming to a space group. TPBMs were proposed in the late 1960's and 1970's as possible microstructures in binary surfactant/water ‘cubic phases’ (Luzzati et al. Nature 220:485 (1968); Luzzati et al. Nature 217:1028 (1968); Lindblom et al., J. Am. Chem. Soc. 101(19):5465 (1979)),and in ternary surfactant/water/oil cubic phases (Scriven, L. E., Nature 263:123 (1976)) (cubic phases are also known as ‘viscous isotropic phase’ liquid crystals). This has been fairly well established for certain binary cubic phases (Longely et al., Nature 303:612 (1983); Rilfors et al., Biochemistry 25(24):7702 (1986)), but until this disclosure, demonstrated with less certainty in the case of ternary cubic phases (Anderson, D. M., Ph.D. thesis, Univ. of Minnesota (1986); Fontell et al., Acta Chem. Scand. A40:247 (1986); Rilfors et al, Biochemistry 25(24):7702 (1986)). TPBM's have also been demonstrated in phases of cubic symmetry occurring in block copolymers (Alward et al., Macromolecules 19:215 (1986); Hasegawa et al., Macromolecules (1986). Described herein is the first polymeric microporous membrane with a highly-branched, triply-periodic network of submicron pores, which has been produced by radical chain polymerization of the oleic component (e.g. methyl methacrylate) of a ternary surfactant/water/polymerizable oil cubic phase.

### “Binary” and “Ternary”

In this description, it should be noted that when the terms “binary system” or “ternary system” are used, they are not meant to exclude systems in which additional components are present but do not affect the development of the desired phase-segregation. For example, components may be present in such small relative quantities that the system is equivalent to a binary or ternary system for the purposes of this invention. Furthermore, one component may consist of sub-components which present nearly identical phase characteristics or which together present a single phase characteristic without departing from this invention. Thus, for example the definition includes a ternary hydrophobe/water/surfactant system whose water portion is a 50-50 mix of water and denatured water and/or whose hydrophobic component is a mix of sub-components which segregate substantially together under the fabrication conditions to be applied.

The procedure used to produce the first example began with a mixture of 1 gm of the surfactant didodecyldimethylammonium bromide (DDDAB; the registry number of DDDAB is 3282-73-3), 1.4 ml of distilled water, and 0.26 ml of methyl methacrylate (MMA) which had been purified by vacuum distillation and to which had been added 0.004 gm/ml of azobisisobutyro-nitrile (AIBN). The mixture was stirred vigorously with a magnetic stir bar in a capped vial (when styrene was used instead of MMA, stirring had to be very gentle). After a few minutes magnetic stirring became impossible because of high viscosity, which together with optical isotropy as checked by observation between crossed polarizing lenses indicate a cubic or ‘viscous isotropic’ phase. At approximately the same volume fractions but with alkanes such as decane or dodecane, cubic phases have been reported by Fontell et al. (Fontell et al., Acta Chem. Scand. A40:247 (1986)) and by the present author (Anderson, D. M. 1986 Ph.D. thesis, Univ. of Minnesota), verified in both cases by small angle x-ray scattering. After equilibrating for a week at 23C, the mixture was smeared onto the end of the plunger into a 1.5 mm i. d. x-ray capillary. After loading and sealing of the capillary, the sample remained clear and optically isotropic. The optical isotropy of cubic phases is due to the equivalence of the three principle directions; other liquid crystalline phases are birefringent.

The capillary was then placed in a photochemical reactor having four UV lights, emitting radiation at 350 nm. The sample was exposed for 36 hours, to bring about radical chain polymerization of the MMA via the decomposition of AIBN into initiating radicals. By the end of this time the sample was opaque white in appearance.

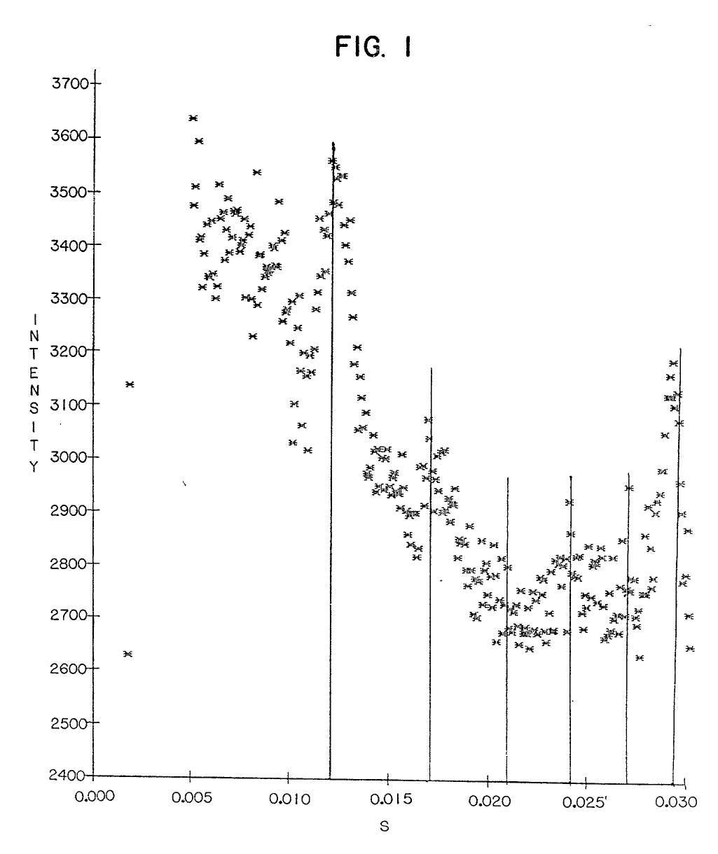

The sample was first examined by Small Angle X-ray Scattering. A Kratky small-angle camera equipped with a position-sensitive detector was used, with tub power set at 1000 watts, and data collected for five hours. The result is shown in FIG. 1, and it is clear that distinct Bragg peaks are recorded.

This verifies that the sample has long-ranged periodic ordering. In FIG. 1 are indicated the theoretical peak positions for a body-centered cubic space group, Im3m, and it is seen that the theoretical peaks are represented by the data.

Recent self-diffusion measurements on DDDAB/water/dodecane cubic phases at approximately the same composition (Fontell et al., Acta Chem. Scand. A40:247 (1986)) indicate that the cubic phase is bicontinuous. This was also the conclusion of the present author, with decane as oil (Anderson, D. M. Ph.D. thesis, Univ. of Minnesota (1986)) that SDS micelles can be swollen with monomeric styrene, and with perceptible change in diameter after polymerization.

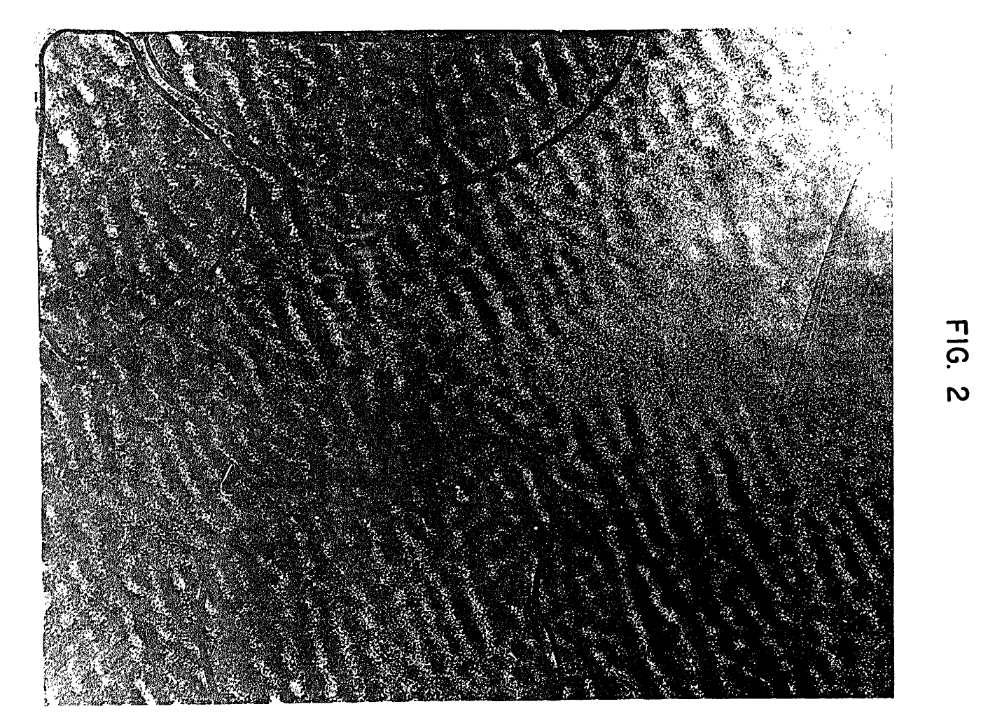

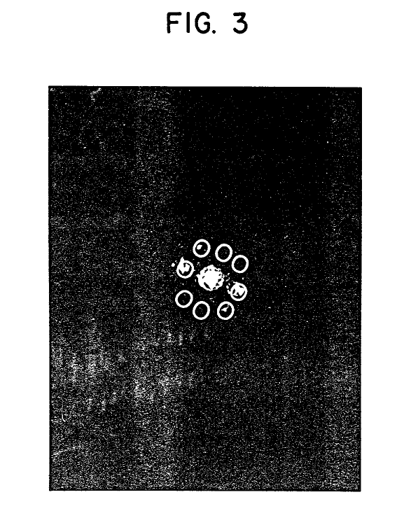

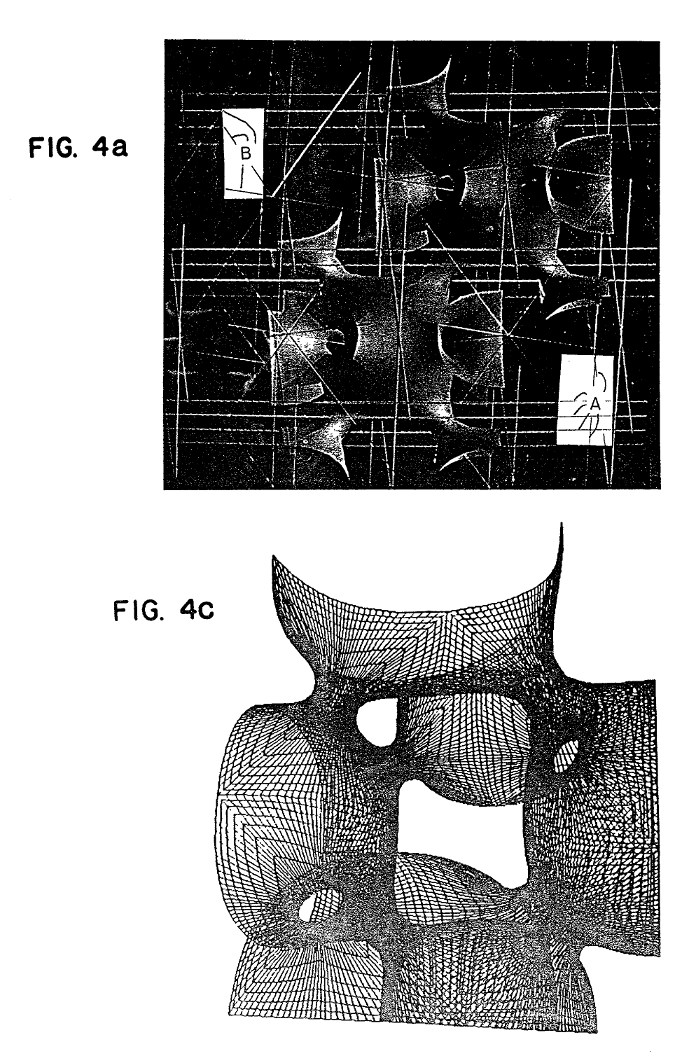

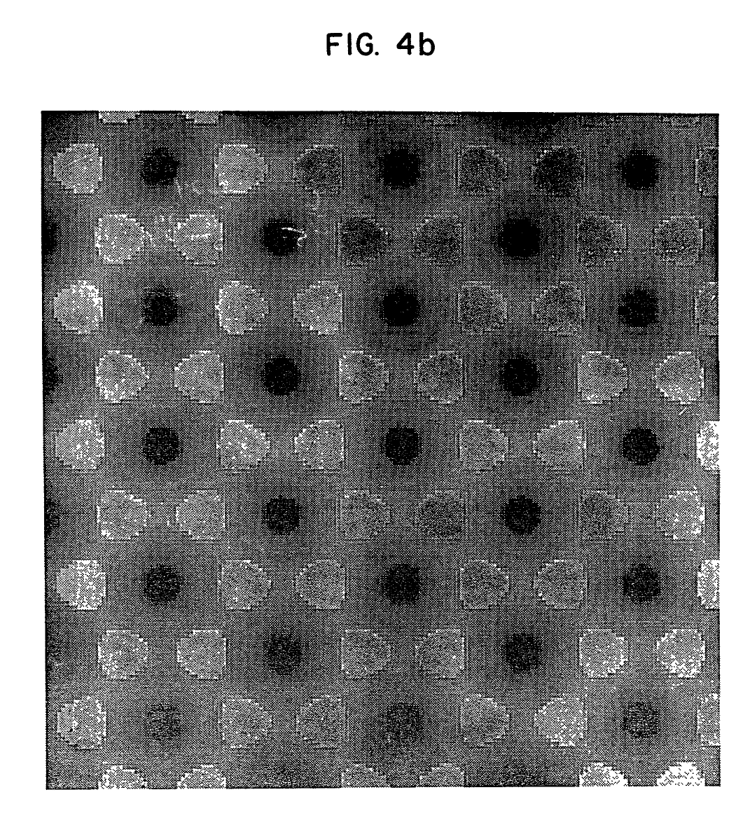

A portion of polymerized sample was dried in a vacuum oven, ultramictrotomed, and examined with an electron microscope. The forces of surface tension on drying would be expected to deform the porous PMMA structure, as would the stress induced by the microtome blade. In spite of this, the electron micrograph in FIG. 2 (magnification 1,000,000×) clearly indicates regions of period order, and this is substantiated by FIG. 3 which is an optical transform of the negative used to make FIG. 2. Cubic symmetry is indicated in FIG. 3 by the eight spot diffraction pattern. FIG. 4 shows a theoretical model of a TPBM of Im3m symmetry that was discovered by the present applicant (Anderson, D. M., Ph.D. thesis, Univ. of Minnesota (1986) see also Nitsche, J. C. C., Arch. Rat. Mech. Anal. 89:1 (1985)), the region lying on the same side of the surface as the graph A in FIG. 4a should be envisioned as being occupied by the surfactant tails and the MMA, with the region lying on the same side of the surface as the graph B containing the water and counterions, and surfactant polar groups located near the dividing surface; after polymerization, the PMMA forms a solid matrix where the MMA was located, this matrix being threaded by the graph A. The (111) projection in FIG. 4b provides a good representation of the ordered regions in FIG. 2.

The same structural model was used to explain SAXS peak positions and relative intensities for a cubic phase with decane as oil, in the present author's thesis (Anderson, D. M., Ph.D. thesis, Univ. of Minnesota (1986)). Since the model represents a bicontinuous structure, it is consistent with the high self-diffusion rates measured for the same phase (Fontell et al., Acta Chem. Scand. A40:247 (1986)), and with the high viscosity of the sample. This high viscosity plays an important role in preventing rearrangement of the microstructure during polymerization.

The fact that the polymerized sample can be dried and microtomed and observed under the electron beam is proof in itself that the MMA has indeed polymerized into a continuous polymeric matrix, because the microtoming was done at room temperature and MMA is a liquid at room temperature. Further proof was provided by the following experiment. The X-ray capillary was broken open and the contents put in methanol, which is a solvent for MMA but a precipitant for polymerized MMA (polymethyl methacrylate, or PMMA). In the 1.5 mm i.d. capillary, the sample was 23 mm long, so that its total volume was 40.6 cubic mm. This 23 mm section of capillary was broken up in a large volume of methanol. Since water and DDDAB are very soluble in methanol, these two components, as well as any unpolymerized MMA, were able to pass through a filter paper. However, the PMMA and the glass from the broken capillary are not soluble and did not pass through. The broken glass and the white precipitate that were stopped by the filter paper were found to have a total weight of 0.008 gm. The weight of 23 mm length of glass capillary is 0.004 gm, so that the amount of precipitate was 0.004 gm. Since the density of MMA is 1.014 gm/ml, and that of both water and DDDAB is 1.00, the mass of MMA in the 40.6 cubic mm of sample investigated should have been 9.7% of that sample, which corresponds to 0.004 gm, as observed. Note that since MMA increases in density by 20% on polymerization, the volume fraction of PMMA in the capillary is only 8%. Yet the PMMA is continuous as evidenced by its integrity; a single connected piece has remained intact floating in methanol for many weeks.

The opaque white appearance of the porous polymer arises from the fact that the microcrystallite sizes are on the order of the wavelength of light, and exhibit tremendous multiple scattering due to the large refractive index difference between the matrix, which is PMMA (n=1.4893 at 23C), and the other subspace, which is either water (n=1.33) or void (n=1 for vacuum, and approximately 1 for air), depending on whether or not the membrane has been dried. It is well known that cubic phases often have large microcrystallites, as evidenced by spotty x-ray patterns (e.g., Balmbra et al., Nature 222:1159 (1969)), and in some cases even by optical microscopy (Winsor, P. A., in Liquid crystals and plastic crystals. vol. 1, G. W. Gray and P. A. Winsor eds, Ellis Harwood Ltd., Chichester (1974)), so that 500 nm would not be unusually large.

It is, of course, possible to dry the membrane without subjecting the matrix to forces of surface tension, by a process known as critical point drying. In general this is not necessary, however, because the membrane can be kept wet at all times during use.

The membrane type described herein can be fabricated in many ways. As mentioned above, bicontinuous microstructured phases (of cubic symmetry) occur also as equilibrium morphologies in block copolymers, and chemical erosion of one component can result in a similar membrane type. It has been shown (Alward et al., Macromolecules 19:215 (1986)) that the lattice size scales as the ⅔ power of the molecular weight of the copolymer, if the ratio of the two components is fixed. Since anionic polymerization reactions can produce star-block copolymers with extremely narrow molecular weight distributions, fabrication with copolymers provides a means of producing a membrane of prescribed pore size.

The surfactant DDDAB was chosen for the fabrication of this first example because it has been shown to form bicontinuous phases with many oil-like compounds: hexane through tetradecane (Blum et al., J. Phys. Chem. 89:711 (1985)); alkenes (Ninham et al., J. Phys. Chem. 88:5855 (1984)), and cyclohexane (Chen et al., J. Phys. Chem. 90:842 (1986)); brominated alkanes (present author, unpublished); and mixtures of alkanes (Chen et al., J. Phys. Chem. 90:842 (1986)). However, an extensive study of cubic phases (Rilfors et al., Biochemistry 25(24):7702 (1986)) indicates that bicontinuity is the rule rather than the exception. Therefore there exists a wide variety of ternary systems that provide possible paths to the type of membrane described herein. In addition, binary water/polymerizable surfactant cubic phases could provide another route, although it is doubtful whether porosities of 90% could be obtained in this manner, since binary cubic phases generally occur near 50/50 surfactant/water. Zadsadsinski, J. A., Ph.D. Thesis, Univ. of Minnesota (1985), has synthesized a polymerizable phospholipid, and produced lamellar phase liquid crystals which retained the same periodic spacing after polymerization, as checked by electron microscopy Zadsadsinski, J. A., Ph. D. Thesis, Univ. of Minnesota (1985), and by SAXS (present author, unpublished). Alternatively, a similar end product can be obtained by chemical alteration of a cubic phase formed from block copolymers, as mentioned above. One aspect of the present invention relates to the final product irrespective of the particular process used to derive it. The polymerization of the oleic component of a binary or ternary hexagonal phase, or chemical alteration of a block copolymer cylindrical phase, to yield a membrane with a doubly-periodic arrangement of cylindrical pores, would also be an useful modification of the present invention, as would the polymerization of a microemulsion containing a polymerizable component (for the definition of a microemulsion, see Danielsson et al., Colloids and Surfaces 3:391 (1981).

Other modifications of the process could produce membranes with special properties. For example, proper choice of monomer which forms an ionomer on polymerization would result in a membrane with electrically charged tunnels. Or the monomer could be chosen to form a conducting polymer on polymerization. Or if the matrix were made with opposite ion-selective properties on its two sides (as should be possible in principle with ternary cubic phases using a polymerizable surfactant, since one side of the surfactant-laden interface is polar while the other is nonpolar), then a bipolar membrane with a great deal of surface area would be obtained. In other words, in some cases two distinct, interwoven but disconnected porespace labyrinths are created, each of which is continuous, highly regular, highly branched and interconnected with itself, each having globally uniform effective pore size; the distinct porespace labyrinths being separated by a continuous stabilized dividing wall, the wall having two distinct surfaces, each surface facing one respective porespace labyrinth. The distinct surfaces may be given different properties. Another possible means of achieving the same end would be to form a cubic phase using a triblock copolymer. Thus, in addition to providing a range of pore sizes that overlaps with that provided by zeolites but extends to much larger sizes, the new membrane type provides the possibility of high porosity, high coordination number, triply-periodic porous media with either nonpolar or polar characteristics.

### MATERIALS AND PROCESS VARIATIONS

There are many potential processes and combinations of materials that could produce polymeric membranes with triply-periodic, submicron porespaces from thermodynamically stable or metastable bicontinuous triply-periodic phases. Possible routes to the fabrication of such a membrane will now be discussed, with an eye toward different membrane applications and the membrane characteristics called for by each. These routes fall into two general classes:

1) polymerization or solidification of a component or components of a surfactant-based triply-periodic fluid phase; and

2) chemical degradation of one or more blocks in a multiblock or graft copolymer-based triply-periodic phase.

There are some important similarities between these two approaches as well as distinctions; for nonionic surfactants can be made which have as few as 20 carbons (see Kilpatrick, P. K., Ph.D. Thesis, Univ. of Minn. (1983) for a discussion of the minimum carbon number for these amphophilic alcohols to be true surfactants), or with molecular weights of thousands when they are referred to as block copolymer polyol surfactants (Vaughn et al., J. Am. Oil Chemists' Soc. 28:294 (1951)), and it is possible that there is a continuum of bicontinuous cubic phases with increasing surfactant- molecular weight that at low Mw yield membranes after a polymerization reaction, and at high Mw yield membranes on the removal of other component(s). Following a discussion of the two classes, methods will be discussed for fabricating triply-periodic ionomeric membranes by similar means or by modifications of neutral membranes of the type described.

Finally, a hybrid process will be discussed in which a membrane formed by a type 1) process (or less likely a type 2) process) is infiltrated with a polymerizable material that is then polymerized, after which the original material is eroded away. In such a process the initial membrane would be of low porosity, say 10%, so that a 90% porosity membrane would finally result, and there would be a great deal of freedom in choosing the final monomer since the triple-periodicity would already be imposed by the initial membrane. A further variation of this process would be to infiltrate with a polymer that is above its melting temperature, and then allowing the polymer to solidify; the polymer that formed the original matrix would then be dissolved away by a method such as those discussed in this section.

### Class 1) Processes

In the first general class of procedures, a surfactant or mixture of surfactants is needed, which may or may not be polymerizable, and except in the case of a binary polymerizable surfactant/water mixture, another nonaqueous, usually oil-like or at least hydrophobic component which must be polymerizable if the surfactant is not. Since the working definition of a surfactant is an amphophil which is capable of cooperativity such as that needed to form a liquid crystal, any amphophilic compound or mixture of compounds that can form a triply- periodic fluid phase together with water and/or another nonaqueous component would have to be considered a surfactant, whether or not that title or some other title such as cosurfactant, amphophil, block copolymer or alcohol were traditionally used for the compound or mixture (recall that cubic phases are considered ‘liquid crystals’ by convention). For example, recent work in Sweden [Guering and Lindman 1983] has shown that bicontinuous microemulsions can be formed with alcohols that are normally used as cosurfactants. Also, work in that same group (Lindman, private communication, 1986) has shown that bicontinuous phases can be formed without water, using water substitutes; because the same is probably then true of bicontinuous cubic phases, and because it should be possible to form bicontinuous cubic phases without any water-like component such as with a binary surfactant/oil mixture, water should not be considered essential to the process although it will nearly always be involved (it is interesting that there has been nearly as much work done on surfactant/oil/pseudo-water microemulsions as on binary surfactant/oil liquid crystals, largely because of the long equilibration times necessary in the latter case).

Another possible variation of process type 1) would be to form a bicontinuous triply-periodic phase with a surfactant, water, and a polymer above its melting point. Once the phase has been annealed it would be brought down below its melting temperature and the solidified polymer would then exhibit triply-periodic porosity. Such a variation of the process would allow a much larger variety of polymers since they could be synthesized beforehand under any desired conditions. The applicant has done work to [Anderson et al. 1987] in which a calculation of the thermodynamics of bicontinuous cubic liquid crystal morphology is compared with that of the competing morphologies—lamellar, normal and inverted hexagonal, and normal and inverted discrete cubic phases—to predict phase behavior based on certain molecular parameters. The dominant geometry-dependent energies are the so-called curvature energy, which results from the packing of the surfactant molecules at the hydrophilic/hydrophobic interface, and the entopic energy of stretching or compression of the surfactant tails, the two energies also considered dominant in a qualitative discussion by Charvolin, J., J. de Physique 46:C3-173 (1985). The publication will indicate that the bicontinuous cubic phase structure should be expected for a wide variety of systems, because such structures can satisfy curvature requirements while simultaneously keeping stretching energies small. For example, for the family of constant-mean-curvature surfaces (which minimize area under the constraint of a given volume fraction) with the double-diamond symmetry (space group Pn3m) [see Anderson, D. M., Ph.D. thesis, Univ. of Minnesota (1986)), the author has shown that the standard deviation in the distances which the surfactant molecules must reach is only 7% of the average distance. Furthermore, it is known that addition of oils to surfactant/water mixtures can change phase behavior by relieving stretching energy costs (Kirk et al., J. Physique 46:761 (1985)), so that bicontinuous cubic phases should be expected to arise on the addition of a third component, as in the case of DDDAB/water.

As mentioned elsewhere in this disclosure, polymerizable surfactants have been synthesized (Zadsadsinski, J. A., Ph.D. Thesis, Univ. of Minnesota (1985)), and liposomes made with the surfactant in water showed no change in structure on polymerization, as measured by both x-ray diffraction and electron microscopy. The particular surfactant synthesized was a double-tailed phospholipid, with each tail containing one polymerizable double bond. Recently a great deal of interest has arisen in the chemical and biological sciences in the idea of using polymerizable surfactants to study surfactant microstructures. As more types of polymerizable surfactants become available and more is learned about using them, the choices of materials available for fabricating a membrane of the type described herein from binary polymerizable surfactant/water triply-periodic phase will continue to broaden. It is now firmly established that phospholipids form bicontinuous cubic phases (Longley et al., Nature 303:612 (1983); Lindblom et al., J. Am. Chem. Soc. 101(19):5465 (1979); Hyde et al., Z. Krist. 168:213 (1984); for a review see Rilfors et al., Biochemistry 25(24):7702 (1986)). A membrane formed by polymerizing such a cubic phase would be zwitterionic.

Bicontinuous cubic phases have also been formed with a variety of ionic surfactants. In fact the first proposed bicontinuous cubic phase was in a binary soap system, potassium laureate/water (Luzzati et al., Nature 215:701 (1967)). Other examples of binary bicontinuous cubic phases formed with anionic surfactants are: sodium laureate, and relatives with other chain lengths (Luzzati et al., Nature 220:485 (1968)); potassium octanoate, and with other chain lengths; and sodium ethylhexyl sulfosuccinate (Aerosol OT)/water (Lindblom et al., J. Am. Chem. Soc. 101(19):5465 (1979)). An example of a binary bicontinuous cubic phases with cationic surfactants is dodecyltrimethyl ammonium chloride/water (Bull et al., Mol. Cryst. Liq. Cryst. 28:155 (1974)). It has also been long known that many soaps, such as the strontium and cadmium soaps, form single-component cubic phases in which the hydrocarbon and ionic regions are each continuous [Luzzati et al., Nature 215:701 (1967); Luzzati et al., Nature 220:485 (1968)). Calcium p-ethyl-w-undecanoate forms such a structure at room temperature (Spegt, P. A., Ph.D. thesis, Univ. Strasbourg (1964)). Such a structure is to be considered bicontinuous in that the hydrocarbon and ionic groups in the anhydrous crystal are normally dispersed in such a way that either the polar groups or the hydrocarbon tails are segregated into discrete domains. Chemical attack on one of these moieties could yield a triply- periodic microporous solid, with either polar or nonpolar channels depending on the nature of the chemical erosion.

While all of the well-established bicontinuous triply-periodic phases are in fact of cubic crystallographic symmetry (in equilibrium; viz., in the absence of stress forces), there is no reason to believe that triply-periodic structures of other symmetries such as tetragonal, hexagonal, orthorhombic or other could be found. Although it has never been demonstrated with scientific rigor, a bicontinuous phase of tetragonal symmetry, space group I422, was proposed by Luzzati et al., Nature 217:1028 (1968)). In fact, triply-periodic minimal surfaces, of the type invoked in the modern treatment of bicontinuous liquid crystals, having three-dimensional noncubic space groups are discussed by Schoen, A., Nasa Technical Note TN D-5541 (1970), and in the applicant's thesis (Anderson, D. M., Ph.D. thesis, Univ. of Minnesota (1986)). The ‘R’ phase proposed by Luzzati et al. has not been substantiated but if such a structure did exist it would be well represented by the triply-periodic minimal surface of hexagonal symmetry discovered by Schwarz (Gesammelte mathematische Abhandlungen, Springer, Berlin, 2 vols. (1890)) and called H′-T by Schoen (Nasa Technical Note TN D-5541 (1970)), or by a surface of constant, nonzero mean curvature of the same space group and topological type [see Anderson, D. M., Ph.D. thesis, Univ. of Minnesota (1986)). Other models of bicontinuous structures, satisfying the very strong constraint of a constant-mean-curvature interface (the area-minimizing configuration), which are triply-periodic but have noncubic space groups, are presented in the author's thesis.

It should not be surprising that binary surfactant/water cubic phases have shown the ability to solubilize various hydrophobic or amphophilic components. The cubic phase in the 1-monoolein/water binary system has been shown to solubilize diglycerides (Larsson, K., Z. Phys. Chem. (Frankfurt am Main) 56:173 (1967)), protein, and cholesterol up to a molar ratio of 1:3 with monoolein. Interestingly, a bicontinuous cubic phase in the dioleoylphosphatidyl glycerol/water system can actually solubilize the anesthetic dibucaine (Rilfors et al., Biochemistry 25(24):7702 (1986)). DDDAB and water can solubilize up to 11% dodecane in a bicontinuous cubic phase, and also styrene and methyl methacrylate as shown herein, as well as other alkanes (Fontell et al., Acta Chem. Scand. A40:247 (1986)). The soap sodium caprylate with water forms cubic phases with a variety of organics solvents including haptene, decane, and p-xylene (Balmbra et al., Nature 222:1159 (1969)). And a bicontinuous cubic phase has been found in the ternary sodium octanoate/octane/water system (Rilfors et al., Biochemistry 25(24):7702 (1986)). Thus there are substantiated examples of ternary bicontinuous cubic phases with zwitterionic, cationic, and anionic surfactants.

Bicontinuous phases also occur in ternary phase diagrams as islands which don not contact the binary surfactant/water edge—that is, they cannot be obtained by addition of a third (usually oleic) component to a binary cubic phase. This is easy to understand, in that removal of the third component forces the surfactant tails to reach to regions far from the hydrophilic/hydrophobic dividing surface, regions that could otherwise be filled by the third component (Kirk et al., J. Physique 46:761 (1985)). Thus no cubic phase occurs in the DDDAB/water binary system, even though the addition of only a few percent oil can yield a bicontinuous cubic phase.

It is quite possible that very inexpensive yet effective surfactants, produced from vegetable oils, will soon become available. Acylated ester sorbitol surfactants have recently been made using lipase enzymes in organic solvents such as pyridine (Klibanov, A. “Enzymatic processes in organic solvents”, presentation at U. Mass. Amherst, Feb. 20, 1986), and surface tension and emulsification experiments showed a high degree of surfactant behavior, higher in fact than analogous synthetic surfactants. In view of the surplus of carbohydrates in the United States, this method may prove to be a very economical source of surfactants in the near future. Since interfacial tensions as low as 0.1 dynes/cm have been measured between hexane and water using such a surfactant, it is likely that fluid microstructures, such as microemulsions, are forming in a narrow interfacial region. It is now generally agreed that bicontinuous microemulsions are responsible for the lowest oil/water interfacial tensions, so that these surfactants appear to have a sufficiently well-balanced HLB to form bicontinuous phases, including perhaps bicontinuous cubic phases.

Block copolymer polyol surfactants were first manufactured under the trade name PLURONIC by BASF Wyandotte Corporation in 1950. Among the epoxides used as the hydrophobic blocks are [U.S. Pat. No. 3,101,374]: propylene oxide, butadiene monoxide, 1,2-butylene oxide, styrene oxide, epichlorohydrin, cyclohexene oxide, tetrahydrofuran, and glycidyl alkyl ethers; these epoxides satisfy the condition that the oxygen to carbon ratio is not greater than 0.4. And among the epoxides used as the hydrophilic blocks are: ethylene oxide, glycidol, butadiene dioxide, all of which have a oxygen to carbon atom ratio at least 0.4. The molecular weight of these surfactants can be as low as 767 (‘PE 71’) or can be in the thousands. As mentioned above, the ethoxylated alcohol C12E8 is of low molecular weight but is a true surfactant (Kilpatrick, P. K. Ph.D. Thesis, Univ. of Minn (1983)). Therefore there is a variety of chemical units, and a wide range of molecular weights that can yield these types of surfactants, and there exist at least three means by which such a surfactant could be used to obtain a membrane of the present type: a) a cubic phase could be formed with a polymerizable third component (or second component if water is unnecessary) and this component polymerized; b) the surfactant itself could be made polymerizable; or c) if the molecular weight of the block copolymer surfactant were high enough, the copolymer could provide the membrane matrix, after removal of one of the blocks by chemical erosion or of one or more additional components such as the water and or a third component, which might not call for any chemical erosion. The key point about the tremendous range of molecular weights over which the polyol surfactants are available is that the pore size of the resulting membrane can be controlled over a very large range, possibly into the range of thousands of Angstroms.

In the third part of this section are discussed possible methods for converting a neutral membrane of the present type into an ion-exchange membrane, but another possible means to achieve the same end would be to choose a monomer that on polymerization would yield the desired ion-exchange characteristics. Polymethacrylic acid and polyacrylic acid are weak-acid cation-exchange polymers, for example, and since methyl methacrylate (which is quite polar) is easily incorporated into the DDDAB/water cubic phase, it is possible that the same process could yield an ion-exchange membrane.

Plasma is another means by which polymerizations could be carried out in cubic phases, and it is known that hydrophobic monomers such as 4-picoline and 4-ethylpyridine can become hydrophilic polymers on plasma polymerization.

Photoinitiation by, for example, ultraviolet light is a very inexpensive means to polymerize a monomer, and also versatile, so that if volatile components were needed the mixtures could be protected from evaporation losses by materials transparent to UV light—such as quartz if thick walls were necessary (which is unlikely since photoinitiation is usually done at atmospheric pressure) or ordinary glass if thicknesses are not large and the UV wavelength is kept at or above 350 nm.

In the actual production of membranes, polymerization by photoinitiation will be much simpler and quicker than in the main example detailed in this disclosure because thicknesses will be on the order of microns rather than millimeters.

It is important to stress that the surfactant should be recoverable from the membrane in a simple post-polymerization step for recycling, using a solvent for the surfactant which is a not a good solvent for the polymer as was done with methanol in the main example. Since the UV light need only penetrate micron-thick layers and since the photoinitiator can be chosen to be much more sensitive to UV light than the surfactant, and since the reaction can be done at room temperature and pressure, the polymerization reaction should have little effect on the surfactant. Another important characteristic of this general process type is that, because cubic phases are equilibrium phases and are extremely viscous, transient conditions that might affect other fluid microstructures (such as low viscosity, temperature-sensitive microemulsions) have much less effect—as evidenced by the retention of cubic lattice ordering after polymerization in the main example—making the fabrication process flexible and reliable. Thus there is no reason why class 1) processes should be limited to polymerization by photoinitiation; initiation could be by thermal decomposition, redox, radiations such as neutrons, alpha particles or electrons, plasma as mentioned above, or even electrolysis (Pistoia et al., J. Polym. Sci. Polym. Chem. Ed. 17:1001 (1979)). It is even feasible for a condensation polymerization to be performed, if the condensate is something like water or a short-chained alcohol that would be incorporated into the water phase or the surfactant-rich interface. From the standpoint of the stability of the finished membrane, it should be remembered that addition polymers generally have greater thermal and chemical stability than condensation polymers.

Particularly in view of the variety of surfactants capable of forming bicontinuous cubic phases, there is a wide range of monomers that have potential for the basis of the matrix material in process type 1). The two monomers that have proven particularly successful are styrene and methyl methacrylate. Thus polar (PMMA) and nonpolar (PS) membranes have been produced. Both PMMA and PS are very inexpensive, about $0.30-$0.60 per pound. As discussed elsewhere, the same surfactant DDDAB forms bicontinuous phases also with alkanes, cyclohexane, brominated alkanes, mixtures of alkanes and, significantly, alkenes. The latter is significant because the presence of carbon double-bonds makes these polymerizable, such as with a Ziegler-Natta catalyst; note that such a polymerization would yield a stereospecific polymer. Isotactic and syndiotactic PMMA can be prepared with Ziegler-Natta catalysts, and these have been used in dialysis membranes (Sakai et al., in Ultrafiltration membranes and applications, A. Cooper, ed., Plenum, N.Y. (1980)). Isotactic polystyrene has high thermal and hydrolytic stability as well as stiffness. Other relatives of PMMA provide potential materials for process 1) membranes, some offering particular advantages for certain membrane applications. As mentioned above, methacrylic acid is a relative of MMA that is the basis of some weak-acid cation exchange membranes, as is acrylic acid. Often copolymers with divinyl benzene are used. Another member of the acrylic family, polyacrylonitrile, is commonly used in UF membranes (usually as a copolymer with a few mole percent of another monomer such as styrene or vinyl chloride), and these are resistant to both hydrolysis and oxidation.

Polyvinyl chloride (PVC) and its copolymers (such as with vinyl acetate) are free-radical initiation polymers which are also important membrane materials. PVC exhibits high stiffness and good solvent resistance, and is inexpensive. Chlorinated PVC is denser and exhibits greater thermal stability. Copolymerization with propylene yields a polymer that is resistant to most acids, alkalis, alcohols, and aliphatic hydrocarbons.

Later in this section we discuss other classes of monomers that can be used in type 1 processes.

The variation of the process described above in which a polymer above its melt temperature—or at least at high enough temperature to allow sufficient mobility for a triply-periodic phase to form—is incorporated into a surfactant-based phase, and the polymer then solidified into a membrane matrix, could be used to form a triply-periodic membrane with other polymeric materials that are particularly well suited for certain membrane applications. Among these are:

polyethylenes (as in Celgard membranes), and its copolymers such as with vinyl acetate or acrylic acid, or with propylene as in polyallomers;

fluorinated polymers, such as polytetrafluoroethylene, polyvinylidine fluoride, polyfluoroethylene-propylene, polyperfluoroalkoxy, and polyethylene-chlorotrifluoroethylene. Membranes made from perfluorinated ionomeric polymers are now more important than all other ionomeric membranes combined;

polyorganosiloxanes (silicones);

cellulose and its derivatives, including cellulose nitrate, cellulose acetate and triacetate (in a binary surfactant/polymer cubic phase, since cellulose is extremely hydrophilic);

polyamides, which fall into three subclasses, fully aliphatic, aromatic, and fully aromatic, all three of which have examples that are used as membrane materials. Membranes made from polypiperazines exhibit long lifetimes and chlorine resistance;

other special polymers, such as polyparaphenylene sulfide which is melt-processable and can readily be made conducting (Baughman et al., in Proceedings of the symposium on membranes and ionic and electronic conducting polymers, May 17-19, 1982 Case Western Reserve University, The Electrochemical Society, New Jersey (1983)). Such processes are now more feasible in light of new research (Charvolin, J., J. de Physique 46:C3-173 (1985)) on naturally-occurring surfactants with very good thermal stability. Alternatively, the polymers could be solidified inside the pore space of a triply-periodic (low porosity) membrane made of dissolvable material, avoiding the necessity to subject the surfactant to elevated temperatures.

### Class 2) Processes.

In this class of procedures, a triply-periodic phase is prepared which incorporates a multiblock or graft copolymer, using a solvent or temperature elevation, or both, to enhance mobility, and one or more of the blocks form(s) the membrane matrix after elimination of one or more component(s) to form the pore space. In general this appears to be a more difficult process than type 1) processes because of the following reasons:

a) expensive anionic polymerizations have been necessary thus far to produce copolymers sufficiently monodisperse to form triply-periodic phases;

b) because of the inherently lower mobility of copolymers relative to small-molecule surfactants, more involved annealing procedures employing solvents and elevated temperatures are generally needed;

c) dissolving away one labyrinth of solidified polymer while leaving another labyrinth intact is generally difficult; and

d) porosities higher than 70% will be extremely difficult to obtain, and higher than even 40% will be difficult, with this process.

On the other hand, in this method, as in some of the variations of type 1) processes discussed above, the polymerization reaction(s) can be carried out before the formation of the triply-periodic phase. The study of the morphologies of phase-segregated block copolymers is quite young and has not received a great deal of attention. Therefore very little is known about the occurrence of bicontinuous cubic phases in block copolymers. Generally speaking, however, the situation is in many ways simpler than in surfactant systems where electrostatic interactions between surfactant head groups play a dominant role in determining microstructure. In diblock copolymers, on the other hand, the morphology is essentially determined by the immiscibility of the two covalently bonded blocks, so that two diblock copolymers, with the same volume ratio between the two blocks, should to first order be expected to exhibit the same morphology. To a large extent this has been borne out by the diblock and star-block copolymers whose phase behavior has been studied; at nearly 50:50 volume fraction ratios between the two blocks, lamellae generally are present; at high volume fraction ratios, approximately 80:20 or higher, spheres are present; and in between one finds cylindrical morphologies or bicontinuous cubic morphologies, the latter generally restricted to a narrow range near 30:70. This is also the situation predicted by simple (Inoue et al., Presentation at the International Symposium on Macromolecular Chemistry, Toronto, Canada, Sept. 5, 1968) and more sophisticated theories (Leibler, L., Macromolecules 13:1602 (1980); Ohta et al., Macromolecules 19:2621 (1986)), except that these theories were developed before the discovery of bicontinuous block copolymer morphologies and so did not include these possibilities. Thus, the proof of the existence of bicontinuous cubic phases in star-block (Thomas et al., Macromolecules 19(8):2197 (1986)) and in linear diblock (Hasegawa (1987)) copolymers indicates that these phases will be found in a variety of copolymers as studies of morphology continue, now that the identity of the phase has been established.

Further indication that bicontinuous cubic phases should be found in many block copolymers near 70:30 volume fraction ratio lies in the fact that the ‘double diamond’ bicontinuous cubic morphology has been found at both: i) 30% polystyrene outer blocks, 70% polyisoprene inner blocks in 6-18 arm star-block copolymers; and ii) 30% polyisoprene outer blocks, 70% polystyrene inner blocks (i.e., interchange PS and PI); as well as in iii) 34% polystyrene, 66% polydiene linear diblock copolymers. It is in fact the case that in the third example, the discoverer (Hashimoto) had many years ago taken SAXS and electron microscopy data on the phase and not understood the data, until hearing of the work by Thomas et al. Thus it is likely that triply-periodic morphologies occur in many block copolymers, although it appears that they are generally confined to narrow volume fraction ranges near 70:30. It also appears that the polydispersity of the copolymer cannot be too high: the studies on bicontinuous cubic phases in copolymers have thus far used only highly monodisperse copolymers (polydispersity indices less than 1.05) prepared by anionic polymerizations, and it is quite possible that such well-ordered morphologies are the result of well-ordered materials!

The preparation of block copolymer TPBMs with polystyrene/polyisoprene is described in (Alward et al., Macromolecules 19:215 (1986) and Thomas et al., Macromolecules 19(8):2197 (1986)). The choice of solvent and annealing temperature will of course depend on the polymers used, but the general procedure will be similar. What has not yet been carried out, however, is the leaching out of one phase to create voidspace. Methods and materials will now be discussed for such a process.

If one of the blocks, call it block A, contains double bonds in the backbone, such as the rubbers polyisoprene and polybutadiene, and the other block(s) do(es) not, then ozonolysis could provide a means to leach block A. Following treatment with ozone to form ozonides, the decomposition of the ozonides can be accomplished in a number of possible ways: 1) they can be oxidized, for example using a reduced platinum oxide catalyst; 2) they can be decomposed by steam distillation, using an alcohol solvent, in which case no reduction step is necessary; 3) a modification of 2) is to carry out the ozonolysis in an alcohol such as methanol; 4) reducing agents such as zinc dust in acetic acid can be used.

If the block A is chosen to be radiation sensitive, with the other block(s) insensitive, then in view of the small thicknesses of membranes it should be feasible to destroy block A with radiation and leave a relatively intact polymer matrix. Many polymers suffer degradation on intense radiation, and in fact some are used in the electronics industry, for example, as negative photoresists due to this property. PMMA is radiation sensitive, for example, and although it has not been tried, PMMA/polyisoprene or polybutadiene copolymers should be capable of forming bicontinuous cubic phases, in analogy with polystyrene.

As in nucleation-track membranes, a combination of ionizing radiation and chemical etching could be used that would be selective to one block. It is known that for every polymer (in fact every substance) there is a lower limit of heavy ion mass below which tracks are not produced. For example, tracks are produced in cellulose nitrate by hydrogen ions, while Mylar (polyethylene terephthalate) requires ions at least as heavy as oxygen. A diblock copolymer selectively tracked in one component could then be immersed in acid or base to etch away pores. Olefin metathesis is another reaction that is used today to degrade polymers. Again what is required is the presence of double bonds in the polymer backbone, so that as in the discussion of ozonolysis the PS/PI block copolymers would be archetypical candidates. In general such reactions require more critical conditions than ozonolysis, and also ozone being a very low MW gas means that penetration through the porespace would be more easily accomplished with ozone. Attack of one block by other chemical means such as with acids is of course possible. For example, polyesters and polyethers can be cleaved under acidic conditions.

Thermal decomposition, by choosing one block with a lower ceiling temperature, is another possible means, which could circumvent the need for reactive chemicals. For example, poly-a-methyl styrene undergoes an unzipping reaction above 50C.

Biodegradable polymers are another possibility, currently of interest because of their application in controlled drug-release. Homopolymers and copolymers of lactic acid and glycolic acid are examples that have been examined for use in the body, but many other biodegradable polymers have been investigated for applications to the dispensing of herbicides and insecticides.

In the last part of this section, possible methods are discussed for modifying neutral polymers to form ionogenic polymers, but of course another possible means to produce an ionomeric membrane is to use a type 2) process in which the block(s) that will determine the membrane matrix is (are) ionogenic. Ionomeric membrane polymers that could be copolymerized with a leachable polymer include random copolymers with etylenically unsaturated monomers containing ionogenic groups. The first such example was a copolymer of acrylic acid with ethylene incorporating inorganic ions [Surlyn]. Other examples include ethylenically unsaturated monomers containing sulfonate groups copolymerized with acrylonitrile, and monomers containing quaternary ammonium or weakly basic groups. Ionomeric step reaction polymers include polyurethanes with quaternary ammonium groups in the backbone, in which case these ionomers are also called ionene polymers. Among other ionomeric materials that could form blocks in a block copolymer are those modifications of neutral polymers discussed in the last part of this section. Generally speaking, the chemistry of block copolymerizations and linking reactions has seen considerable growth in recent years, and in the future the availability of block copolymers with desired block properties will increase.

In order to understand and predict the occurrence of triply-periodic bicontinuous morphologies in block copolymers, the applicant has developed a statistical mechanical theory that compares the free energies of the known morphologies in the strong-segregation limit. The theory combines the results of Ohta and Kawasaki (Ohta et al., Macromolecules 19:2621 (1986)). and de la Cruz and Sanchez (de la Cruz et al., Macromolecules 19:2501 (1986)), and is an improvement over the approach of Ohta and Kawasaki in that the exact expression for the static structure factor of a star diblock copolymer (equation 28 in de la Cruz and Sanchez), which includes the linear diblocks treated by Ohta and Kawasaki as a special case (n=1), is used in the computation rather than an approximation (as in equation 3.19 of Ohta and Kawasaki). Furthermore, and of prime interest here, the triply-periodic bicontinuous morphology named the ‘ordered bicontinuous double-diamond’ in Thomas et al. (Thomas et al., Macromolecules 19(8):2197 (1986)) has been evaluated in the free energy comparison. The calculation will now be described for the free energy competition between bcc spheres, hexagonal-packed cylinders, lamellae, and ordered-bicontinuous double-diamond, as a function of composition, arm number and molecular weight; the model used for the double-diamond morphology is one of the constant-mean-curvature-interface structures of the ‘D’ family discovered in the applicant's thesis. The general approach was introduced by Leibler (1980), and his predictions of phase behavior and scattering curves in the weak-segregation limit have been shown to agree well with experiments (Mori et al., Polymer J. 17:799 (1985)).

Beginning with equation 3.14 in Ohta and Kawasaki, the bilinear term in the free energy was evaluated using Fourier transforms, where the integration becomes a summation because the Fourier transform of a periodic function consists of delta—functions at reciprocal lattice vectors. The static structure factor that is equation (28) in de la Cruz and Sanchez (which reduces to equation 3.15 of Ohta and Kawasaki when n=1) was used in its exact form; the q² term remains the same as that in 3.19 of Ohta and Kawasaki for any arm number n, while the function s(f) of 3.19, which gives the constant in the asymptotic behavior for large q, can be calculated to be:

s(f)=1−(n−1)(1−f)/2+f(1−f)(n−3)/2.

These two terms were subtracted off from the expression (28) since they are included in the short-range free energy contribution in the analysis of Ohta and Kawasaki. The long-range contribution is then evaluated by summing, over all reciprocal lattice vectors, the product of the resulting expression with the square of the form factor;

$${F}_{L} = \frac{1}{2 C} \frac{\sum }{{{q}_{\sim}}_{i}} ( {S}^{-} 1 ( qi ) ) - \frac{1}{2 {N}^{2} f ( 1 - f )} [ \frac{{q}_{j}^{2} N}{2} + \frac{S ( f )}{f ( 1 - f )} ] ) {Ψ}_{qi} {Ψ}_{-} qi$$

the corresponding term in Ohta and Kawasaki's formulation is the A(f) term in equation 3.20 that is multiplied by the square of the form factor. Clearly it is a considerable improvement to use the exact expression (3.15 for linear diblocks, and (28) in de la Cruz and Sanchez for stars), rather than the approximation 3.19 which matches the exact expression only to an accuracy of 4% and has the wrong asymptotic behavior for large q; this can easily be accomplished since the integral becomes a summation in reciprocal space and the series converges rapidly. Note that this approach is equivalent to Ewald's method in the limit of large G. After the summation to yield the long-range free energy contribution, the surface area per unit volume yields the short-ranged contribution just as in Ohta and Kawasaki (using their approximation that the interfacial tension is the same for all morphologies), and the total energy is minimized over the lattice parameter.

It remains to describe the calculation of the form factor for the double-diamond structure; the form factors of spheres, cylinders, and lamellae are all well-known. By using the divergence theorem, the volume integration can be reduced to an integration over the surface Hosemann et al., Direct analysis of diffraction by matter, North-Holland Pub., Amsterdam (1962)):

$${Ψ}_{q} = ⟦ v {e}^{i} {q}_{\sim} \cdot {r}_{\sim} {d}^{3} {r}_{\sim} = \frac{i}{{q}^{2}} ⟧ \partial V {n}_{\sim} \cdot {q}_{\sim} {e}^{i} {q}_{\sim} \cdot {r}_{\sim} ⅆ A$$

The surface in the finite element solution is represented by triangular patches (much as in a geodesic dome), and because the normal direction is fixed over a given triangle in space, this integral can be done analytically over every patch. The surface integral in equation (2), evaluated over a triangle in which the x-y-z coordinates of the three vertices are given by (x1,y1,z1), (x2,y2,z2), and (x3,y3,z3) is exactly:

$$\frac{M}{{q}^{2}} \{ [ COS ( a + b ) - COS ( a + c ) ] / b ( c - b ) - [ cos a - cos ( a + c ) ] / b c$$

where a =(X₁, Y₁, Z₁)·{tilde under (q)}⊃b=(X₂, Y₂, Z₂)·{tilde under (q)}−a, c=(X₃, Y₃, Z₃)·{tilde under (q)} M=|(X₂−X₁, Y₂−Y₁, Z₂−Z₁) X (X₃−X₁, Y₃−Y₁, Z₃−Z₁)|.

A fundamental patch of the surface was represented by 800 such triangular patches; a unit cell of surface can be broken down into 24 identical fundamental patches. The from factor calculated in this way is mathematically exact for the structure so represented. The applicant's thesis contains demonstrations of the accuracy of the finite element representation of these constant-mean-curvature surfaces.

Below are reproduced the computer codes used for 1) the computation of the form factor from the surface (Program FORF); and 2) the summation in reciprocal space and final computation of the total free energy for the candidate structures (Program CRUZ). The bcc spheres were omitted because they are favored only for small values of the volume fraction f (<0.22), and the double-diamond occurs at values of f (or of 1−f) near f=0.3.

The results of the theory are now given for a volume fraction of f=0.644 (the volume fraction for the surface with mean curvature equal to 1.6), as a function of arm number; this is the volume fraction of the inner or core blocks of the star. There is also a dependence on molecular weight (which is not predicted by Ohta and Kawasaki because of their use of the approximated structure factor), and this is described by the parameter N which is the product of the square of the Kuhn step length with the number of Kuhn steps in a single arm, divided by 6. In the experiments of Thomas et al. (i Macromolecules 19(8):2197 (1986)), the unit cell was on the order of 30 nm, and the statistical Kuhn length on the order of 1 nm, so that in dimensionless units this length is 0.33, and since the polymer index was about 160, a good value for this parameter is 0.003. The free energies of the candidate morphologies, as a function of arm number, are as follows:

| arm number | D-Diamond | Lamellar | Cylindrical |
|---|---|---|---|
| 1 | 1.107211 | 1.076124 | 1.074017 |
| 2 | 1.060160 | 1.049548 | 1.048806 |
| 3 | 1.042949 | 1.041448 | 1.041374 |
| 4 | 1.037309 | 1.039309 | 1.039511 |
| 5 | 1.037388 | 1.039869 | 1.040130 |
| 6 | 1.040689 | 1.041883 | 1.042074 |

These energies are in the same units as those in Ohta and Kawasaki. Thus it is seen that double-diamond is calculated to occur at higher arm numbers, as was observed in the experiments of Thomas et al.

The key to these results is that no assumptions were made about the specific chemistry of the copolymer, such as the interaction parameter, as long as this interaction parameter is large enough for the strong-segregation to assumption to be valid. Thus the ordered bicontinuous double-diamond morphology is predicted to occur in a wide variety of block copolymer systems. It should be emphasized again that the statistical mechanical treatment underlying this theory has been shown to agree well with experiments.

Conversion of Neutral Polymers to Ionomers.

The commercial importance of ionomeric polymer membranes has stimulated research on methods of converting neutral polymers to ionomers, both before the formation of a membrane and as a post membrane formation step. Methods of incorporating ionomers into membranes with triply-periodic submicron porespaces have been described in this section and include:

a) conversion of a neutral polymer membrane produced by polymerization of a component of a small-molecule triply-periodic phase via a process of type 1);

b) formation of a triply-periodic phase incorporating an ionogenic polymer above its melting point, followed by subsequent solidification of the polymer;

c) infiltration of a (low porosity) triply-periodic membrane with either an ionomer (above its melt temperature), or a monomer that can be polymerized, and modified if necessary, to form an ionogenic polymer; and

d) formation of a triply- periodic morphology with a block or graft copolymer one component of which is ionomeric.

The two most important classes of ionomeric polymers in membranology are the styrene-type and perfluorinated ionomers, and the primary focus of this part will be on these, although other classes of ionomers may be found to be compatible with the types of processes described herein. Reactions for grafting ionogenic polymers or oligomers to neutral polymers will be briefly discussed; such reactions are the subjects of investigations in present-day polymer research and promise to open up new possibilities for the grafting of ionogenic polymers in a post membrane formation process. In addition, such graft copolymers might be used as the basis for type 2) processes, for recent evidence (Hasegawa (1986)) indicates that graft copolymers can form bicontinuous cubic phases.

Styrene polymers, and copolymers with, for example divinyl benzene and/or ethyl vinyl benzene, are excellent starting materials for the formation of ionomers, because of the reactivity of the aromatic rings for chloromethylation, nitration, and particularly sulfonation. Such polymers can be converted to strong acids by sulfonation with sulfuric or chlorosulfonic acid, and this can be followed by conversion to the sodium form by addition of a slight excess of alkali. Weak-acid cation exchange polymers can be made by with acrylic or methacrylic acids, as mentioned above. These reactions can be performed after the formation of the membrane with the neutral polymer.

Strong-base anionic-exchange polymers can also be produced from styrene-based polymers or copolymers in a post membrane formation step. Chloromethylation by methyl chloromethyl ether, followed by amination with a tertiary amine, yields strong-base polymers even in pure polystyrene. Amination of the same chloromethylation product with primary or secondary amines yields weak-base anion-exchange polymers. Redox membranes, which are oxidation and reduction agents lacking actual charged groups, can be produced by addition polymerization of styrene, divinyl benzene, and esterified hydroquinone.

Perfluorinated ionomers are presently the most important cation-exchange membrane polymers, primarily because of their strength and to chemical stability. As an example of the possibilities of production of these types of ionomers, consider starting with a copolymer of tetrafluoroethylene and perfluoro 3,6-dioxa-4-methyl-7-octene-sulfonyl fluoride. The sulfonate groups can be converted to the sulfonic acid form by nitric acid, after which oxidation in n-butyl alcohol followed by hydrolysis with sodium hydroxide yields a polymer suitable for use as an electrolysis membrane. Reaction with vaporous phosphorous pentachloride followed by treatment with triethylamine and immersion in a solution of water, dimethyl sulfoxide and potassium hydroxide, or by treatment with aqueous ammonia, also yield ionomeric polymers suitable for electrolysis. Polyol surfactants can be subjected to reactions that induce an ionic character. The terminal hydroxyl groups can be converted to various functional groups (Lundsted et al., in Block and Graft Copolymerization, vol. II, R. J. Ceresa, ed., John Wiley and Sons, New York (1976)), such as to a halide and subsequently to a tertiary amine by reaction with a substituted amine. This in turn can be converted to an amine oxide, by reaction with hydrogen peroxide, or to a cationic quaternary surfactant by reaction with an alkylating agent. Polyurethane can be obtained by reacting with diisocyanate. Anionic surfactants can be produced by addition of epichlorohydrin and sodium sulfite, or by reaction with an oxygen-containing acid or acid anhydride. And cationic surfactants can also be produced from block copolymeric surfactants by reaction with ethylene or propylenimine, or by methylation.

A great deal of recent research has focused on conducting polymeric membranes. Electroactive polymer films have been produced by electropolymerization of aromatic heterocyclic compounds (Diaz et al., in Proceedings of the symposium on membranes and ionic and electronic conducting polymers, May 17-19, 1982 Case Western Reserve University, The Electrochemical Society, New Jersey (1983)). Highly conducting membrane polymers have been produced by iodine-doping (Schechtman et al., in Proceedings of the symposium on membranes and ionic and electronic conducting polymers, May 17-19. 1982, Case Western Reserve University, The Electrochemical Society, New Jersey (1983)), and by electrochemical reactions (Huq, R. et al., in Proceedings of the symposium on membranes and ionic and electronic conducting polymers, May 17-19, 1982 Case Western Reserve University, The Electrochemical Society, New Jersey (1983)); in fact, polyacetylene can be reduced or oxidized to compositions that have the electronic properties of metals.

Grafting of neutral but potentially ionomeric materials onto neutral membrane polymers, particularly as a post membrane formation step, is another proven source of ionomeric membranes. Polyacrylate ester can be grafted onto cellophane, and subsequently hydrolyzed to produce a weak-acid cationic-exchange membrane. Similarly polystyrene has been grafted onto polyethylene and sulfonated, to form a strong-acid cationic-exchange membrane. For post membrane formation grafting reactions, the creation of free radicals on the pore surfaces to act as initiation sites for polymerization of added monomers is attractive, in that monomers could diffuse easily to these sites. Free radicals can be produced for grafting sites by peroxides or redox catalysts, or by exposure to electrons, gamma rays or UV radiation.

### Industrial Applicability

As previously mentioned, the past 20 years has seen tremendous growth in the applications of polymeric membranes, not only in filtration—microfiltration (MF), ultrafiltration (UF), and hyperfiltration or reverse osmosis (RO)—but also in a variety of other areas such as fuel cells and batteries, controlled-release devices as for drug or herbicide metering, dialysis and electrodialysis, pervaporation, electrophoresis, membrane reactors, ion-selective electrodes, and as supports for liquid membranes, to name some important areas. Furthermore, modification of neutral polymer membranes can yield ionomeric or ‘ion-exchange’ membranes which are finding increasing application in many chemical, electrochemical, filtration and even biochemical processes. In many applications the availability of a membrane of the type described herein with precisely-controlled porespace and high porosity represents a significant technological advance.

Traditionally membranes have been associated with filtration processes for purification or concentration of fluids, or recovery of particles as in the recovery of colloidal paint particles from spent electrolytic paint particle suspensions, and the very important application of recovering of lactose-free protein from whey. The use of reverse osmosis and electrodialysis in removing trace pollutants from industrial waste streams is increasing each year, as the cost of these processes is often less than other alternatives (Spatz, D. D., in Synthetic membranes, vol. II, A. F. Turbak, ed. ACS Symposium Series, Washington D.C. (1981)); because these processes are being applied for waste treatment in agricultural, chemical, biochemical, electrochemical, food, pharmaceutical, petrochemical, and pulp and paper industries, the development of this technology will have a significant impact on the environment.

The earliest, and still the most frequently mentioned, use of RO (also known as hyperfiltration) is in the desalination of salt water and brackish. Desalinated water obtained from RO of seawater could be an important solution to the fresh water shortages that are projected over the next few decades. The literature on desalination by RO is extensive. From the point of view of the present invention, the two characteristics that distinguish the RO membrane from UF and MF membranes—namely smaller pore size (less than 10 Angstrom) and lower porosity—would result from the polymerization of the surfactant of a binary surfactant/water bicontinuous cubic phase. As discussed earlier, the very concept of bicontinuity first arose in experiments on binary surfactant/water cubic phases, and there are now many such binary cubic phases believed to be bicontinuous, most of which occur near 50% volume fraction water and with channel diameter less than 4 nm. Alternatively, RO membranes of intermediate porosity, roughly 70%, would result from chemical erosion of one component of a block copolymer cubic phase of low molecular weight. In his discussion of RO membranes, Kesting (Synthetic polymeric membranes, John Wiley and Sons (1985)) lists narrow pore size distributions as the first criteria for an effective membrane.

Reverse osmosis is finding new applications every year. RO and UF are being investigated (Drioli et al., in Synthetic membranes, vol II, A. F. Turbak, ed. ACS Symposium Series, Washington D. C. (1981)) for the treatment of must and wines without the addition of sulfur dioxide, which is routinely added to remove certain enzymes that would otherwise cause an oxidized taste. The concentration of tomato juice by RO has been applied on a semicommercial scale, and results in enhanced taste and color over conventional processes (Ishii et al., In Synthetic membranes, vol. II, A. F. Turbak, ed. ACS Symposium Series, Washington D.C. (1981)). A recent study (Farnand et al., in Synthetic membranes, vol. II, A. F. Turbak, ed. ACS Symposium Series, Washington D.C. (1981)) has shown that RO can also be used to separate inorganic salts from nonaqueous solvents such as methanol; the latter solvent is of particular importance in that methanol is being investigated as an alternative fuel.

As pointed out by Spatz (in Synthetic membranes, vol. II, A. F. Turbak, ed. ACS Symposium Series, Washington D.C. (1981)), there is in reality no fine line between Ro membranes and UF membranes, but rather the pore size in the UF membrane is generally larger, so that the UF membrane does not reject small molecule salts as does the RO membrane. A typical UF membrane will reject over 99% of the organics over 200 molecular weight and over 98% of monosaccharides such as dextrose and glucose. Size fractionation is the basis of many UF processes, and narrow pore size distributions are often critical, as in hemofiltration for the treatment of renal failure (Kai et al., in Synthetic membranes, vol. II, A. F. Turbak, ed. ACS Symposium Series, Washington D.C. (1981)); the increased discrimination of hemofiltration with UF membranes over that of hemodialysis with respect to the rejection of solutes larger than uric acid has been proposed as the reason for the success of hemofiltration for hemodialysis-difficulties patients.

Ultrafiltration is of importance in the separation of viruses, which by virtue of the fact that they are much smaller than bacteria generally pass through microfiltration membranes, unless the latter are treated so as to be positively charged (Brock, T. D., Membrane filtration: a user's guide and reference manual, Science Tech, Inc. Madison, Wis. Data on page 57 courtesy of Oxoid Ltd., Basingstoke, England (1983)). This leads to failure when contaminants neutralize the charge, after which the retention or passage will depend only on the pore size (Raistrick, J., Proceedings of the World Filtration Congress III, London (1982)). The virus known as human T-lymphotropic virus III (HTLV-III; also called human immunodeficiency virus or HIV) is a sphere of diameter roughly 1,000 Angstroms, now believed to be responsible for the disease AIDS as well as other neurological disorders and perhaps even the cancers. The potential importance of a membrane of the type disclosed herein is demonstrated by the fact that some hemophiliacs developed AIDS after receiving infusions of a plasma preparation called Factor VIII, which had been passed through a filter that was fine enough to remove bacteria but not virus particles (Gallo, R. C., Scientific American, January 1987).

In dialysis, solute permeates through a membrane from a more concentrated to a less concentrated solution; thus it differs from UF in that in the latter the solute flux is coupled to the solvent flux. The dialysis of blood to remove urea and creatinine from uremia patients, known as hemodialysis, is believed to be presently the largest single application of membranes to separations. Dialysis is also used in the pharmaceutical industry to remove salts, in the rayon industry, and in the metallurgical industry to remove spent acids. Since dialysis membranes are generally very finely porous—with molecular weight cutoffs of around 1,000—the present invention could be applied in these areas; in the case of hemodialysis, where human suffering is involved, advantages offered by a more precisely controlled membrane could well justify a higher cost, if the present invention were more expensive than the extruded cellulose hydrogels that are presently used.

Another medical application for membranes is in controlled drug-delivery systems. The simplest description of these is that a drug is imbibed into the pores of a membrane, and released slowly so as to approximate a constant concentration over time in the body (zero-order release), or a concentration that fluctuates in response to physiological conditions (first-order release). In some cases biodegradable polymers are used, such as lactic acid and glycolic acid homopolymers and copolymers. In the case of first-order systems for the release of insulin in the treatment of diabetes, a glucose-sensitive membrane is being investigated (Kost, Y. “Internally and externally-controlled drug-release membranes”, presentation at U. Mass. Amherst, Jan. 15, 1987) in which the enzyme glucose oxidase is immobilized in a poly-N, N dimethylamino-methyl methacrylate/poly-HEMA copolymer. so far the membrane has shown the ability to release ethylene glycol in response to glucose concentration, but porosity of greater than 50% is required to release insulin. Some other drugs which are being investigated for membrane release are nitroglycerine, progesterone, and epinephrine, to name only a few examples. The importance of high porosity and therefore high concentration in the membrane, and of well-defined pores has lead to the use of phase-inversion membranes prepared by the so-called thermal process; the diameters of the cells in these membranes are between 1 and 10 microns, with porosities of roughly 75%. Membrane metering devices are potentially of great utility in the release of other effectors such as fragrances, insecticides, and herbicides.

Polymer UF membranes provide supports for liquid membranes, in which the liquid is immobilized in the porespace of the solid microporous membrane by capillarity. The immobilized liquid membrane offers the advantages over solid membranes of higher diffusivities, higher solubilities, and in many cases very high selectivity. Concentrated CsHCO₃ aqueous solutions can be use to recover carbon dioxide from gaseous mixtures (Ward, W., in Recent developments in separation science, N. Li, ed., vol. I, CRC Press, Boca Raton, Fla. (1972)). Liquid membranes are also used to recover carbon dioxide from the products of carbon dioxide-based tertiary oil recovery methods, and to remove ammonia from waste water. Immobilized liquid membranes have been proposed for the removal of toxic materials such as dichromate ions from electroplating rinse waters (Smith et al., in Chemistry and water reuse, W. Cooper, ed., Ann Arbor Science Pub., Ann Arbor Mich. (1981)). UF membranes also provide possible supports for so-called dynamically-formed membranes. The homogeneity of such a membrane is highly dependent on the degree of order in the porespace of the support; carbon black has been used but due to the presence of large pores; the homogeneity and permselectivity have not been good. The two most important physical characteristics of the most desirable support would be a high degree of order and a pore size less than 1 micron, both of which are satisfied by the present invention. Dynamically-formed membranes can be used to separate small molecules and ions, and have been shown to be effective in the desalination of water (Kraus et al., Desalination 1:225 (1967)).

Chromatography is a separations process that is of great importance in analytical chemistry. In gel-permeation chromatography (GPC), separation of chemical mixtures is based on differences in passage times through a mobile liquid phase filled with porous polymeric particles. Separations on the basis of molecular weight could be enhanced by a polymer with monodisperse pores.

Pervaporation is a membrane-based separations process capable of separating complex azeotropic mixtures. It also circumvents the problem in RO of high osmotic pressures that oppose flux in attempts to concentrate a solute to high purity. Pervaporation has been shown to be capable of separating linear hydrocarbons from olefins, and from branched hydrocarbons (Binning, et al. Ind. Eng. Chem. 53:45 (1961)). Thus interest in membranes with precisely controlled porespaces has arisen in the petroleum industry. Diffusion of the components through the membrane is the rate-limiting step, and thus high porosity and uniform pores are important in pervaporation as well as in the recent modification of the process known as membrane-aided distillation.

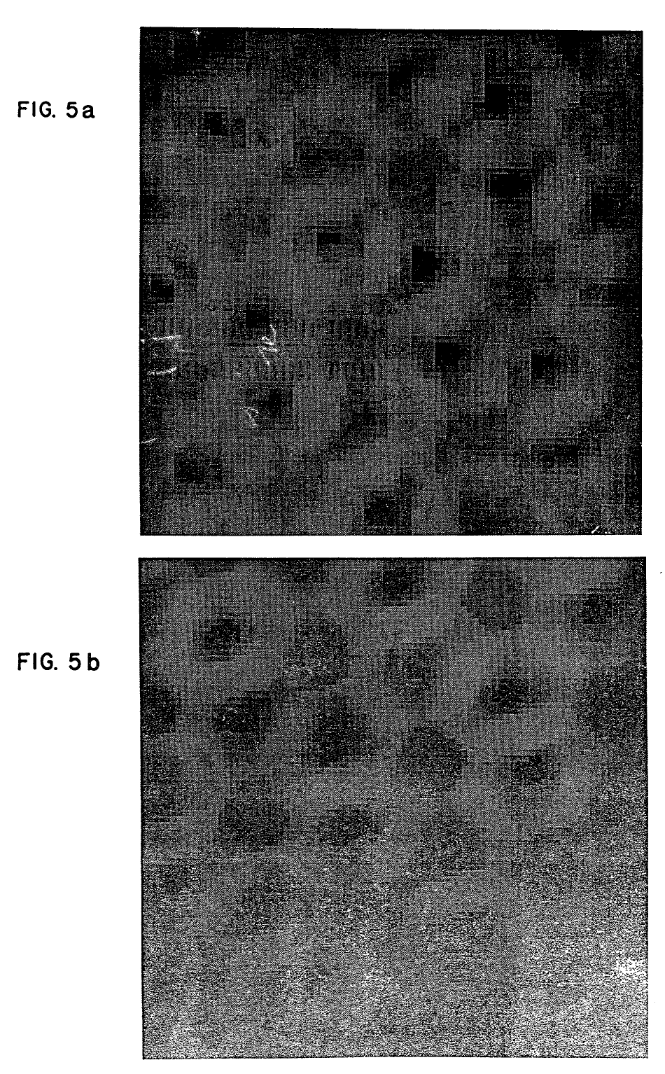

Electrophoresis is a separations process for macromolecules such as proteins which is based on an imposed electric field, where a porous membrane must be used to frustrate remixing via thermal convection. Finely porous membranes such as agarose or polyacrylamide gels with pore sizes on the order of 1,000 Angstroms result in enhanced separation over that of cellulose acetate membranes with pores on the order of 1 micron, due to a combination of both the electrophoretic effect and sieving. Electrophoresis is an important tool today in biological and bioengineering research, and it is anticipated that it will be realized in large scale separations processes, and in three dimensions, in the near future. Certainly in cases where sieving is a significant contribution to the separation, a membrane with triply-periodic submicron pores may be of importance. The applicant has demonstrated (Anderson, D. M., Ph.D. thesis, Univ. of Minnesota (1986)) that the progressions of structures that occur in phases of cubic symmetry should also include structures that consist of interconnected sphere-like domains, which would be the perfect geometry for an electrophoresis membrane. The electron micrograph of FIG. 2, and the model structures in FIG. 4 indeed indicate an interconnected-sphere structure. Also, the model that is to date the best model for the cubic phase occurring in the star-block copolymers of Thomas et al. (Thomas et al., Macromolecules 19(8):2197 (1986)) is based on a surface of constant means curvature from the author's thesis which is shown in the thesis to be very accurately described by interconnected, nearly-spherical domains. At present, studies are underway to determine more precisely the exact shape of the domains. FIG. 5 shows the comparison between a (digitized) electron micrograph of a star-block copolymer cubic phase and the theoretical prediction from the constant-mean-curvature-interface model.

Selective membrane electrodes are chemically-specific probes in which a reference electrode is separated from the test solution by a selective membrane; the species to be detected diffuses through the membrane and reacts so as to produce an ion that is measured by an ion-selective electrodes. A wide variety of membranes is used, including both neutral and ionomeric membranes, and enzymes immobilized in microporous membranes. Selective membrane electrodes are used to detect carbon dioxide in blood and fermentation vats, ammonia in soil and water, sulfur dioxide in stack gases, foods, and wines, sulfur in fuels, nitrite in foods, and hydrogen cyanide in plating baths and waste streams, for some examples.

### Ionomeric Membranes.

Methods have been described herein for fabricating ionomeric, or ‘ion-exchange’ membranes with the triply-periodic porespaces that distinguish this invention. In view of the fact that the surface area of the membrane analyzed earlier is 3500 sq. meters/gram, such a membrane would be of potential impact in the general field of ion-exchange membranes and resins—in particular in applications where precise porespace characteristics are required, such as when ion-exchange or electromembrane processes are enhanced by or combined with sieving. As in the case of neutral membranes, the field of ion-exchange membranes and resins is large and ever-expanding, so that only a brief overview of the applications with respect to the present invention can be given here.

Electrodialysis is the most important electromembrane process, used in the concentration or removal of electrolytes, metathesis reactions, and the separation of electrolysis products. Ion replacement is also important in, for example, citrus juice sweetening where citrate ions are replaced by hydroxyl ions. Electrodialysis for ion-exchange of Na+ to CA+, K+, or Mg+ is being investigated as a source of low-sodium milk. Because the resistance to solvent flow is important in problems of anomalous osmosis and incongruent salt flux, a membrane with uniform pores would enhance the predictability of the process. Although there is debate about the exact origin of anomalous osmosis (Schlogl, R., Z. Phys. Chem. (Frankfurt) 3:73 (1955)), there is some evidence that it is due at least in part to inhomogeneities in the porespace (Sollner, K., Z. Elektrochem. 83:274 (1932)). Also, electrical conductance is lower in heterogeneous membranes than in homogeneous polystyrene-based membranes, for example (Kedem et al., in Industrial membrane processes, AIChE Symposium Series 248(82)4:19 (1986)).

Ion-exchange membranes are used in batteries in part because their electrical conductances are higher than in the silver halides of conventional solid-electrolyte cells. They are also used in fuel cells such as the Bacon cell, in which hydrogen and oxygen are combined to form water with the release of heat and electricity. Efficiencies of these chemical reactions can approach 100%. Because of the high reactivity of hydrogen, the Bacon cell can be operated at relatively low temperatures, opening up the possibility of using an ion-exchange membrane as a solid-state electrolyte. The ideal electrolyte would be permeable to only one ionic species, and if this were to be accomplished or aided by membrane sieving, very uniform pores would be required. In view of this, and of the other advantages offered by membrane electrolytes over metal electrolytes such as small unit thickness, immunity to carbon dioxide impurities in the hydrogen feed, and the ability of the membrane to also serve as the gas separator, the present invention could prove to be the best possible electrolyte in such a cell.

Both neutral and ionomeric membranes of the type described herein could be used in a variety of other reactions, for example by doping the membrane with a catalyst or by controlling a reaction rate precisely by diffusion limitation. The large specific surface, 3500 sq. sites could allow for a greater degree of control than has been possible with prior art membranes.

### Differences from the Prior Art—Statement of Six Advances in Membrane Technology Represented by the Present Invention

1. Because the source of the structure in the present invention is characterized by thermodynamic equilibrium, all cells (pore bodies), as well as all pore throats, are substantially identical in both size and shape, and the sizes and shapes are controlled by the selection of the composition and molecular weights of the components, over a size range which includes that from about 10 Angstroms to about 250 Angstroms pore diameter and in some cases beyond the micron range, and cell shapes which cover a range including that from substantially cylindrical to spherical, and cell diameter to pore diameter ratios which cover a range including that from 1 to 5, and connectivities which cover a range including that from 3 to 8 pore throats emanating from each cell.

2. The porespace comprises an isotropic, triply-periodic cellular structure. No prior art microporous polymeric material, and no prior art microporous material of any composition with pore dimensions larger than 2 nanometers, has exhibited this level of perfection and uniformity.

3. In certain forms of the invention, the microporous polymer creates exactly two distinct, interwoven but disconnected porespace labyrinths, separated by a continuous polymeric dividing wall, thus opening up the possibility of performing enzymatic, catalytic or photosynthetic reactions in controlled, ultrafinely microporous polymeric materials with the prevention of recombination of the reaction products by their division into the two labyrinths, and with specific surface areas for reaction on the order of 10³-10⁴ square meters per gram, and with the possibility of readily controllable chirality and porewall surface characteristics of the two labyrinths.

4. The microporous material exhibits in all cases a precisely controlled, reproducible and preselected morphology, because it is fabricated by the polymerization of a periodic liquid crystalline phase which is a thermodynamic equilibrium state, in contrast to other membrane fabrication processes such as that in Castro et al. which are nonequilibrium processes. (Castro et al. U.S. Pat. 4,519,909.)

5. Proteins, in particular enzymes, can be incorporated into the cubic phase bilayer and then fixated by the polymerization, thus creating a permanent reaction medium taking advantage of the precision of the present invention, and maintaining to the highest possible extent the natural environment of the protein. As shown by K. Larsson et al. (J. Disp. Sci. Tech., 3:61-66 (1982)), a very hydrophobic wheat fraction, gliadin, can be dispersed in the biological lipid (surfactant) monoolein, and a bicontinuous cubic phase formed on the addition of water. Examples of other proteins and enzymes which can be incorporated into bicontinuous cubic phases are reviewed in (B. Ericsson et al., Biochim. Biophys. Acta, 729:23-27 (1983)), and several other examples are detailed below. The present invention presents a stabilized form of such phases.

6. The components can be chosen so that the material is biocompatible, allowing use in controlled-release drug-delivery and other medical and biological applications that call for nontoxicity. Furthermore, in dialysis, immunoadsorption processes, or other blood applications, where traditional membranes such as Cuprophan induce complement activation and collagen membranes activate clotting, membranes made by polymerization of cubic phases can immobilize enzymes (such as protein A) and effect the adsorption of antibodies through a combination of adsorption and size-fractionation, without activating clotting and with less complement activation than even polyacrylonitrite membranes.

### Brief Example of the Significance of the Differences Noted Above

1. Clearly one important application of microporous materials in which the effectiveness is critically dependent on the monodipersity of the pores is the sieving of proteins. In order that an ultrafiltration membrane have high selectivity for proteins on the basis of size, the pore dimensions must first of all be on the order of 25-200 Angstroms, which is an order of magnitude smaller than the smallest pore dimensions of the microporous material described in the patent of Castro et al. In addition to this, as emphasized in that document one important goal in the field of microporous materials is the attainment of the narrowest possible pore size distribution, enabling isolation of proteins of a very specific size, for example. Unless, as in the present invention, the pores are all exactly identical in size and shape, then in any attempt to separate molecules or particles on the basis of size, the effectiveness will be reduced when particles desired din the filtrate are trapped by pores smaller than the design dimension or pores which are oddly-shaped, and when particles not desired in the filtrate pass through more voluminous pores. This is particularly important in hemodialysis and microencapsulation of functionally specific cells.

2. Certain studies of superfluid transitions require microporous materials exhibiting long-range, triply-periodic order. In the Laboratory of Atomic and Solid State Physics at Cornell University, a group lead by Dr. John D. Reppy has been investigating the critical behavior of liquid ⁴He in microporous media (preprint available). Certain theoretical treatments have predicted that the critical exponents characterizing the fluid-superfluid transition are different for disordered than for periodic porous media. The experiments described in the paper now being submitted for publication were performed using disordered media: Vycor, aerogel, and xerogel. The group is now proceeding on to a parallel set of experiments using the ordered microporous medium of the present invention, supplied by the applicant. Thus an early practical use of the present invention is as a scientific standard

3. One cubic phase structure has two enantiomorphous channels separated by a continuous surfactant—or in some cases water—matrix. It is now known that in some such cases, such as the system monoolein/cytochrome/water, these two channels do not have the same composition, most likely due to the fact that the cytochrome, which is chiral, locates in the water network with left-handed screw symmetry. Therefore, if this phase is made with a polymerizable surfactant, then the polymerization creates, remarkably, a chiral membrane filter, with all pores having the same chirality. Purifications involving chiral separations are notoriously difficult and, therefore, expensive, but such a filter could lead to tremendously simpler and more efficient chiral separations.

4. As pointed out in the patent of Castro et al., the microporous material disclosed which is formed through a nonequilibrium process, is subject to variability and nonuniformity, and thus limitations such as block thickness, for example, due to the fact that thermodynamics is working to push the system toward equilibrium. In the present invention, the microstructure is determined at thermodynamic equilibrium, thus allowing uniformly microporous materials without size or shape limitations to be produced. As an example, the cubic phase consisting of 65% dodecyldimethylamine oxide in water is stable over a temperature range of more than 80° C., so that addition of monomer into the water (e.g., acrylamide) or the hydrocarbon component followed by thermal initiation produce uniform microporous materials of arbitrary size and shape. Further, recent work has shown that the DDAB/methyl methacrylate/water cubic phase disclosed above is stable at least to 55° C., and furthermore at least 25% monomeric acrylamide can be incorporated into the aqueous phase, so that polymerization of either the oleic component or the aqueous phase via a thermally initiated polymerization produces uniform microporous materials of arbitrary size and shape. Also, monoolein cubic phase in water is stable from less the 20° C. to over 90° C.

5. Inherent in the present invention is a direct means to incorporate proteins with enzymatic or catalytic activity, for it has been shown that many proteins and enzymes, in particular, are readily entrapped in cubic phases, this being a thermodynamic equilibrium state, and the preparation of such a cubic phase with polymerizable surfactant, or with an aqueous-phase monomer, followed by polymerization would then fixate these proteins forming a stable, reusable reaction or detection medium. To name a single example in the growing field of immobilized enzymes for medical assays, the enzyme glucose oxidase can be used to detect concentrations of glucose in serum, and glucose oxidase can be entrapped in the monoolein/water cubic phase (C. Tilcock et al., Biochim. Biophys. Acta, 685:340-346 (1982)). It is known that the effectiveness, stability, and insensitivity of inhibitors or immobilized enzymes is in general optimized when the enzyme is in an environment which most closely resembles its natural environment, and fixation into a lipid bilayer represents a significant advance in this respect.

6. Cubic phases can be used in controlled-release drug delivery. Polymerized drug-bearing cubic phases provide for controlled-release applications with high stability. The combination of the biocompatibility and entrapping properties of many cubic phases with the increased stability upon polymerization leads to new delivery systems, and even first-order drug release—release in response to physiological conditions—by incorporating proteins and enzymes, as described elsewhere, as biosensors.

A very promising technique should be mentioned in connection with controlled-release applications. Since we can polymerize our samples by light, we can take spherical (say) particles of the cubic phase, and polymerize just long enough to create a polymeric outer coating. This would open up at least three new possibilities. First of all, one can use this to modulate the release rate and profile. Second, consider the following scheme for creating a first-order release material. One can polymerize an outer coating on a particle which would contain glucose oxidase immobilized in a cubic phase. When glucose levels in the blood got high, then this would cause a drop in pH due to the action of the glucose oxidase on glucose. Methods are then known for using a pH change to cause release of insulin. And third, one can encapsulate very large things such as cells, viruses, etc. by surrounding them with cubic phase and then polymerizing; the polymerized-bicontinuous-cubic-phase coating would then control which components would get access to the encapsulated material and which would not. For example, pancreatic islets can be encapsulated and protected from the body's immune system while insulin and glucose could pass freely into the islets. The chemistry of this last example is discussed at more length elsewhere in this application.

### Further Background, Discussion and Example

This section discusses potential applications of the present invention in catalysis, immobilized enzymes, separations, and other areas in greater detail, focusing in particular on applications where the technological advances listed above open up new possibilities which clearly are not possible with prior art microporous materials and in particular with the material described in the patent of Castro et al. As discussed above, the present invention represents a synergistic combination of many previously unattainable qualities in microporous polymeric materials for use in catalysis, including precisely controlled pore size and shape, fixed coordination number, and a biocompatible and highly versatile matrix material, together with high specific surface areas, high porosities, and uniform and selectable porewall characteristics. In actuality, the term ‘biocompatible’ is a considerable understatement, because in the realm of solid microporous materials a polymerized lipid bilayer represents the environment that is closest to the natural environment of the protein-rich lipid bilayer of the living cell; this lipid bilayer is the site of a myriad of biochemical reactions and transport processes, and it is well- established that the optimal environment for the functioning of proteins and enzymes in technological applications is that which most closely resembles the protein environment in vivo.

Furthermore, a remarkable and unique feature of certain forms of the present invention is the presence of two continuous, intertwined but disconnected aqueous networks in the case of a binary surfactant/water cubic phase, or as in the cubic phases described by (Scartazzinin and Luisi (1988)), hydrophobic networks. To date, isotropic microporous materials have been of one of two types; A) the porespace (except for isolated, inaccessible pores) is connected into one labyrinthine subspace, as in the material described by Castro; or B) two distinct labyrinths are present which are very different in porewall characteristics, for instance one polar and the other apolar. The latter type would result from the polymerization of the surfactant in a ternary cubic phase such as the DDAB cubic phase described in the present application; as mentioned above, the present applicant has synthesized a polymerizable analogue of DDAB, so that both of these classes of materials are attainable in the present invention. However, in addition, cubic phases offer the unique opportunity to create a new, third type of microporous polymeric material, displaying exactly two aqueous labyrinths, as present in many biological systems (there in unpolymerized form, of course) such as the thylakoid membranes, the endoplasmic reticulum, and possibly also in the digestion of fats (Patton (1981)). Indeed, some of the potential applications of such a material are suggested by biological processes in plant and animal cells: catalytic reactions, particularly those involving proteins, creation of membrane potentials as in photosynthesis), and separations of high specificity through the fixation of trans-bilayer proteins which facilitate the transport of certain molecules, to name some examples. Other applications do not appear to have precedent in biological processes, such as the separation of enantiomers by the creation of a chiral filter.

### Catalytic Reactions Which Have Been Performed in Micelles

In one embodiment of the present invention, some or all of the surfactant is polymerized and is thus present along the porewalls, making it very straightforward to take advantage of the known catalytic properties of surfactant aggregates. Clearly this is not the case with other microporous materials such as those described in the patent of Castro et al., nor with the other prior materials. In fact because of these catalytic properties, the present invention would be very valuable even if its sole novel feature were a surfactant-lined porewall. Also in such applications the extremely high specific surface area of the present invention, as well as the precisely controlled morphology, are important and valuable qualities. For applications in which the present technology calls for the solubilization of catalysts or coenzymes in micellar phases, it is likely that the same catalysts could also be solubilized in cubic phases, in stable or metastable states.

Micelles are extremely dynamic structures, and in fact the average residence time of a molecule in a micelle is on the order of 0.1 microseconds. Thus in many applications the chemical and structural fixation of the cubic phase by polymerization would be a significant improvement. This is particularly true for case in which the present technology involves continuous nonaqueous solvents and thus inverted micelles, because it is a well-known principle that inverted micelles are more easily disrupted by the addition of solubilizates than normal micelles. In many applications of surfactant aggregates catalysis, the effect of the surfactant is largely due to the electrostatic field present at the head group region. However, in other cases the catalytic action of micelles is crucially dependent on penetration of the substrate into the hydrocarbon core of the micelle (or the aqueous core of the inverted micelle). In such cases a polymerization of the surfactant could interfere with or actually ruin the catalytic potential of the cubic phase. This is not necessarily the case, though, because even bulk polymers are penetrable to many substances, especially when swollen, this in fact being the basis for the use of many polymers in ultrafiltration membranes, of course. Furthermore, the rate of penetration of a substance through a polymerized monolayer or bilayer will obviously be much faster than that through a bulk polymer. Moreover, the bicontinuous nature of the cubic phases of the present invention offers access to both hydrophobic and hydrophilic regions, in contrast with closed micellar aggregates in which the surfactant layer must be crossed in order to access the component in the interior of the micelle.

Another difference between the cubic phase and the micellar phase is the mean curvature of the microscopic interface, generally much smaller in magnitude in the cubic phase, and it is known that the rates and efficiencies of catalysis in surfactant microstructures is dependent on this curvature. For example, the lamellar phase (zero mean curvature interface) has a greater effect on the hydrolysis of procaine than the alkyl betaine/benzaldehyde/water system is reduced most in lamellar phases over micellar.

The use of micelles in catalysis have been reviewed in a book by Fendler. There are some spectacular examples, such as a rate enhancement of five million-fold for the equation of [Cr(C₂O₄)₃]^{3 -}— through the use of octylammonium tetradecanoate micelles. Certain hydrolysis reactions show rate enhancement of more than 20,000 with the surfactant phosphotidylethanolamine, relative of which are known to form bicontinuous cubic phases. Inverted swollen micelles made with Aerosol OT (sodium ethylhexyl sulfosuccinate), octane, and water increase the rate of imidazole-catalyzed hydrolysis of p-nitrophenyl acetate, and in the phase diagram of Aerosol OT/isoctane/water there is a cubic phase region of rather large extent, and this cubic phase is known to be bicontinuous (Fontell et al., Acta Chem. Scand. A40:247 (1986)).

In general, the use of surfactant microstructures in catalysis is an extremely promising area, and substrate specificity is frequently very high. We have just scratched the surface of the potential for phase transfer catalysis. The material of Castro et al. is not suited for such applications, whereas the present invention may represent an important breakthrough in many such applications, particularly where the precise size and shape (and in some cases, chirality) of the pores would enhance the process by rejecting unwanted or non-participating species, or by optimizing the registry between the substrate and catalyst through the pore geometry.

### Photocatalytic Reactions

Water-in-oil microemulsions have been demonstrated to have the ability to provide a reaction medium for coupled redox reactions which mimic the photosensitized electron-transfer processes in photosynthesis, with the surfactant interface effecting the separation of the redox species and thus preventing the thermodynamically favored back-reactions (Willner, Otvos, and Calvin (1981)). In one reaction, the photosensitizer tris (2,2′-bipyridine)-ruthenium (II) (Ru(bipy)3 2+) was dissolved in the aqueous cores of dodecylammonium propionate/toluene/water inverted micelles, along with the electron donor ethyendiamine-N,N,N′,N′-tetraacetate (EDTA); the primary acceptor benylnicotinamide, being amphophilic, located itself at the surfactant-laden interface, but upon oxidation relocated in the continuous organic phase because of charge removal. Once in the organic phase the reduced benzylnicotinamide was converted by an azo dye, 4-dimethylamino-azobenzene, to the surface-active form again, upon reducing the azo dye to a colorless hydrozoa compound. The reduction of the dye was established spectroscopically. Following illumination with light, after four minutes 80 per cent of the dye had been reduced. In a similar manner, a photoinduced oxidation was accomplished, thus determining two complementary half-cells of a model photosynthetic reaction. The eventual goal of such cells is the evolution of hydrogen and oxygen as fuels, and in this respect, it is significant that the oxidation of water by Ru(bipy)3 2+ in the presence of metal oxides has been accomplished (Lehn, Sauvage, and Ziessel (1979)), as well as coupling to hydrogen evolution (Kalyanasundaram and Gratzel (1979)).

The ternary polymerizable surfactant/oil/water cubic phases of the present invention could offer important advantages over the inverse micellar solution utilized in the experiments of Willner et al. Microemulsions are in general very sensitive to changes in temperature and composition, and in any case are rearranging on the scale of microseconds. In particular, inverted micelles have a very short lifetime and are often poorly-defined in contrast to textbook figures which show highly-organized spherical entities. Also, in larger-scale applications where the aim is to establish a continuous flow of reactants and products, and avoid saturation of concentration gradients, clearly the bicontinuous nature of the present invention is advantageous. And when sensitizes which are closer to (or identical with) those occurring naturally are used, then the lower-curvature surfactant interface of the present invention will provide an environment which is more stable and closer to the natural in vivo environment of the sensitizer.

Bicontinuous microemulsions also have continuous oleic and aqueous labyrinths and low interfacial curvatures, but as in micellar solutions the structure is undergoing constant thermal rearrangement on microsecond timescales. Furthermore, the viscosity of a microemulsion is very low, orders of magnitude lower than that of the cubic phases. Therefore, it is not surprising that a recent attempt to polymerize a bicontinuous microemulsion failed to preserve the bicontinuity due to a fundamental change in structure during the polymerization (Candau, Zekhnini, and Durandi (1988)). This appears to be inevitable since polymerization generally takes hours, whereas the time scale for rearrangement of a bicontinuous microemulsion is on the order of nanoseconds. As discussed in greater length above, the more regular packing and higher viscosity of the cubic phase makes fixation of the structure possible via polymerization. The importance of polymerizing the cubic phase in the applications discussed herein is made clear by the fact that most bicontinuous cubic phases occur between other liquid crystalline phases (usually between lamellar and hexagonal or inverted hexagonal phases), so that they cannot tolerate compositional changes in the unpolymerized state. For example, the cubic phases discovered by Scartazzini and Luisi exist only at a very specific water content, for a give organic solvent. Thus, in order to retain the cubic structure in the presence of water or aqueous solution (such as blood), the cubic phase must be polymerized.

As pointed out by Willner et al., their model system is of a fundamentally different type than the photosynthetic system of the thylakoid membrane. Rather than a surfactant monolayer as in the inverted micellar solution, the lipid in the thylakoid membrane is in the form of a bilayer, separating two aqueous compartment, with the stroma side of the bilayer acting as a cathode and the interathylakoid side acting as an anode. Tien (1981) states that the chlorophyll dispersed in the lipid bilayer acts as a semiconductor, in that the absorption of light excites an electron to the conduction band and leaves a hole in the valence band. There are at least twos reasons why the separation of the aqueous phase into two distinct compartment is important in natural photosynthesis: first, as well as providing an appropriate environment for the pigments, the bilayer acts as a barrier to prevent back-reactions; and second, with the two systems of accessory pigments located in distinct parts of the membrane, each electron/hole pair can be generated by two photons, thus providing an upgrading of the photon energy. In the process of the electron-transfer reactions during photosynthesis, a membrane potential of about 160 mV is created across the bilayer, as well as a pH gradient of about-1 pH unit, and the energy of the flow of protons created by this electrochemical proton gradient is used by the transmembrane protein complex ATP synthetase to synthesize ATP from ADP and Pⱼ. In the language of Tien, the semiconducting bilayer separates two highly-conducting aqueous solutions, creating electrical fields of more than 100,000 volts per cm. With these facts in mind, it is clear that the property of one form of the present invention, of dividing space into two aqueous labyrinths, is not an esoteric nor a trivial feature but quite the contrary a feature of potentially great importance. Permeating the bilayer-based cubic phase to fix the structure would generally be important for industrial-scale processes utilizing this property, both to create a solid medium and because the unpolymerized cubic phase is in general very sensitive to changes in temperature and composition. Also, as discussed below transport proteins which would facilitate the processes can be fixated into the polymerized bilayer. The polymerization of the bilayer will not affect the flow of protons and electrons, for example, whereas the flow of other, larger, molecules will be affected, and this may be favorable in some processes and unfavorable in others.

Besides photosynthesis, photocatalytic reactions involving semiconductors have many other potential applications. Photo-Kolbe reactions using semiconductors could be applied to the treatment of waste streams, giving methane and other alkanes as fuels (Tegner (1982)). For example, the purification of waste streams by semiconductor-photocatalyzed (solar) oxidation of CN⁻ and SO₃²— is a spontaneous process. I₂, Br₂, and C₁₂ can be produced over irradiated platinized suspensions of n-doped TiO₂ (Reichman and Bjork (1981)). Hydrogen and oxygen can be formed photochemically on a TiO₂—RuO₂ catalyst using 310 nm light (Kawai and Sakato (1980)).

### Immobilized Enzymes

There are many potential uses of enzymes immobilized in porous materials. Immobilized enzymes offer many advantages over enzymes in solution, including dramatically increased stability in many cases as well as higher activity and specificity, broad temperature and pH ranges, reusability, and fewer interferences from activators and inhibitors. Many of these advantages can be traced to the fact that enzymes in vivo are usually not in solution but instead function in environments for which they are specifically adapted, this very often being in or near a lipid bilayer. Above, it was discussed that the present invention is of potential importance in immobilized enzyme and related applications, such as selective membrane electrodes or ‘biosensors’, controlled-release applications, and extracorporeal circuits. An enzyme immobilized in a polymerized cubic phase of the present invention is in a precisely controlled environment, chemically, geometrically, and electrostatically. As emphasized above, the chemical environment of the enzyme has a crucial effect on the enzyme's activity and stability, and a polymerized bilayer is very close to the natural environment in which the enzyme functions in vivo. The precise geometrical environment provided by the present invention can be utilized to bias the registry between the enzyme and the substrate toward the optimal orientation and proximity, in addition to providing additional control of the chemicals environment through selection on the basis of size. And the electrostatic environment would be very homogeneous due to the strong tendency for charged or zwitterionic surfactant head groups to maintain an optimum separation, this electrostatic environment again being closest to that of the enzyme in vivo, and it is known that the specificity of many enzymes is sensitive to changes in net charge and nearest-neighbor effects (Guilbault (1984)). And on the practical side, another advantage of the present invention in the immobilization of enzymes for biosensors and other applications is the versatility due to the macroscopic physical properties of the cubic phase, namely that it is a viscous liquid crystal and therefore can easily be applied as a cream at the site of application (on the tip of a pH meter probe, for example), and then polymerized.

Studies by Kare Larsson and coworkers at Lunds Universitet have shown that cubic phases, using biocompatible surfactants, can incorporate a wide variety of proteins and enzymes. As mentioned above, there is a large cubic phase region in the phase diagram at room temperature of monoolein/water/lysozyme, extending to over 30 per cent lysozyme. The same lipid with water can also form equilibrium cubic phases incorporating glucose oxidase, a-lactalbumin, soybean trypsin inhibitor, myoglobin, pepsin, bovine serum albumin, conalbumin, and diglycerides. It is known that many biological lipids form bicontinuous cubic phases, including monoelaidin, monolinolein, monopalmitin, monostearrin, monoarchidin, palmitoyllysophosphotidyl choline (PLPC), N-Methylated dioleoylphosphotidylethanolamine (N-methylated DOPE), phosphototidyl choline (PC), egg lysophosphotidyl choline (egg LPC), monoglucosyldiglyceride (MGluDG), diglucosyldiglyceride (DGDG), egg lecithin, glycerol monooleate, dioleoyl monoglucosyldiglyceride (DOMDG), monogalactosyldiacylglycerol (MGalDG), phosphototidic acid with chlorpromazine, lauroyl phosphotidylcholine (LaPC), or replace lauroyl with myristoyl, palmitoyl, stearoyl, oleoyl, or linoleoyl, and polar lipid extracts of Pseudomonas fluorescence and of Sulfolobus solfataricus. Recent work has also shown (Shyamsunder, Gruner, Tate, Turner, and So (1988)) that dioleoylphosphotidyl choline, which does not form equilibrium cubic phases, nevertheless forms metastable cubic phases upon temperature cycling, by repeatedly raising and lowering the temperature above and below the lamellar/inverted hexagonal phase transition and in biological membrane processes, and suggest that other biological membrane-forming lipids might also exhibit metastable cubic phases. Concerning polymerization, a recent review of polymerizable liposomes includes a listing of 10 lipids (not counting variations in chain lengths) which have been polymerized into liposomes (Regen (1988)), as well as 28 other polymerizable surfactants.

Beside polymerizable surfactants, another means to immobilize enzymes within the present invention is to incorporate them into a hydrophobic or hydrophilic polymerizable component. Work in the applicant's laboratory has shown that over 20 per cent of the water in the cubic phase of the C₁₂E₆/water system can be replaced by monomeric acrylamide (AM) and polymerized by UV initiation, and results indicate that the same can be done with the DDAB/dodecane/water cubic phase. Polyacrylamide gels have been shown to have the ability to entrap enzymes, and for many such entrapped enzymes there is very little loss in activity after three months of storage (Hicks and Updike (1966)).

Of course it is possible in the present invention, as in other microporous materials, to immobilize enzymes by more traditional processes such as by absorption or covalent bonding, as a post-membrane formation steps. However, these processes suffer from serious drawbacks. Absorbed enzymes easily desorb upon changes in pH, temperature, ionic strength, etc., seriously limiting their versatility and stability. The main drawback with covalently bonded enzymes is the harsh chemical conditions to which the enzymes are generally exposed during the bonding process, conditions which often lead to seriously reduced activities, and cause significant losses of expensive enzymes. Recently a new process has been found for covalently linking enzymes to collagen, in such a way as to avoid exposing the enzyme to harsh chemical conditions (Coulet and Gautherm (1981)). However, collagen is a powerful platelet antagonist, activating fibrin and leading to immediate clotting, and this makes it totally unsuitable in applications involving contact with blood. Furthermore, neurological complications can result when collagen is used with chemotherapeutic agents, such as Cisplatin (Quinn, Frair, Saff, Kavanagh, Roberts, Kavanagh, and Clark (1988)).

In view of these facts, the present invention could have important research and clinical applications in immunoabsorption processes, which have been tried in cases of systemic lupus erythematosus, rheumatoid arthritis, Guillain-Barre syndrome, pemphigoid, and myasthenia gravis, and represent the method of choice in congenital and acquired hemophilia with inhibitor and goodpasture's syndrome (Freiburghaus, Larsson, Sundqvist, Nilsson, Thysell, and Lindholm (1986)). Such processes are also being investigated for use in the treatment of cancer (Wallmark, Grubb, Freiburghaus, Flodgren, Husberg, Lindholm, Thysell, and Sjogren (1984)), where it has been demonstrated that tumor growth can be inhibited by immunoabsorption. In a prevalent immunoabsorption process, plasma is passed through a column loaded with beads of agarose, to which Staphylococcal protein A (SpA) has been covalently bonded. SpA is known to bind over 90 per cent of the human immunoglobulin IgG, an immunosuppressive factor. The cost of SpA is a major deterrent to its routine clinical use: in Sweden, for example, where much of the research on hemofiltration is conducted, such a treatment costs approximately 200,000 SEK. The present invention could conceivably be used to reduce this cost, because as stated above, the covalent bonding of enzymes involves significant losses, whereas the fixation by polymerization of surrounding lipid does not impose any chemical changes directly on the enzyme. Furthermore, the protein SpA normally functions in a bilayer environment. And other means of enhancing or replacing the SpA adsorption process are made possible by the present invention, such as by removing the immunoglobulin via fractionation, or by enhancing the IgG-removal process by a combination of sieving and adsorption. IgG has a molecular weight of 153,000, which lies well within the range of molecular sizes which can be sieved with the present invention; whereas in the case of the material described by Castro et al., the smallest pore size alluded to is 0.05 microns=500 Angstroms diameter, which is an order of magnitude too large to allow IgG to be separated from the blood components having molecular weights lower than that of IgG.

### Other Blood Applications

Immunoabsorption processes are examples of extracorporeal circuit processes, which also include hemodialysis, membrane plasmapheresis, cardiopulmonary bypass, filtration leukopheresis, and hemoperfusion. A significant complication with these treatments is the activation of complement, causing side effects that are well-known in the field of clinical hemodialysis; fever, sweating, respiratory distress, chest pain, nausea, vomiting, hypotension, and hypoxemia. The complement C5a can lead to pulmonary leuko-embolization which can eventually trigger respiratory distress syndrome (RDS) (Jacob (1980)). Other complications are interleukin-1 production, liberation of blood granulocyte proteases, and the generation of free oxygen radicals. Furthermore, patients undergoing hemodialysis for more than 5-10 years can develop dialysis-induced amyloidosis, in which deposits of amyloid (the primary constituent of which is β2-2-macroglobulin) are present in the joints, synovium, capsula, subchondral bone and vertebral disks, for example; in fact the amyloidosis may be systemic (Bardin, Zingroff, Kuntz, and Drueke (1986)), for small vascular deposits have been demonstrated in rectal mucosa of dialysis patients, as well as in the heart, liver and lungs.

It is now well-established that the characteristics of the dialysis membrane—in particular the selectivity, thickness and adsorption characteristics—are critical in determining the extent of these complications. The pore uniformity and biocompatibility of the present invention could reduce or circumvent these complications. As mentioned above, the present invention opens up the possibility of developing a hemodialysis or hemofiltration technique which would utilize the monodispersity and resulting selectivity on the basis of molecular weight. The membranes used to date in hemodialysis have had wide pore-size distributions. The primary therapeutic effect of hemodialysis appears to be the removal of urea and creatinine, which have molecular weights of 60.1 and 131.1 respectively, and thus should be able to pass through a microporous membrane with pores small enough to reject typical proteins. Thus, application of the present membrane could very well eliminate complications associated with transfer of larger molecules such as complements, antibodies, and other proteins. In general it is clear that the availability of a precisely-controlled membrane with a high degree of biocompatibility could be invaluable in the research and development of hemodialysis treatments aimed at more control over the exact blood constituents whose concentrations are affected. The immediate goal of such studies would be the reduction of side effects which cause suffering and illness in patients undergoing dialysis treatment; the long-range potential benefits could include improved and more affordable treatments for uremia, hemophilia, rheumatoid arthritis, and perhaps even cancer.

In addition, it is known (Ven der Steen (1986)) that polymethylemthacrylate, the polymer comprising the membrane which is served as one of the main examples in the applicant's disclosure, is significantly more biocompatible than the Cuprophan membranes that are currently the most widely used in hemodialysis. The in-vitro complement activation after 240 minutes of hemodialysis was approximately 10 micrograms/ml (c3b, c) using a PMMA membrane, considerably lower than the 75 micrograms/ml measured using a polyacrylonitrile membrane. It is well-established that membrane-induced leukopenia is complement mediated. As discussed above the level of biocompatibility that can be achieved in the present invention is very high, and furthermore since it has been demonstrated that membrane thickness should be kept to a minimum in order to minimize complement activation (Van der Steen (1986)), the high degree of uniformity of the present invention could be important in allowing reductions in thickness without reductions in efficiency or selectivity.

Microencapsulation of cells such as pancreatic islets followed by implantation in the body is an attractive alternative to organ transplants, which is now the fastest growing area in diabetes research. The islets are protected from the body's immune system by encapsulation using a semipermeable membrane which allows the free diffusion of insulin and glucose into and out of the islets, but isolates the islets from the antibodies and lymphocytes of the host. Considering that the molecular weight of insulin is 11,466, while that of a typical IgG-fraction antibody is about 150,000, and making a crude estimate of the effective ‘diameter’ D of the protein by setting (pi/6)D³ equal to the volume of the protein, we see that this ‘diameter’ is about 33 Angstroms for insulin and 78 Angstroms for the antibody. These estimates are, of course, very crude in that, for example, the shape of IgG is more of a T-shape, but qualitatively the conclusion is that the pore size requirement is of very monodisperse pores, preferably with significantly less that a 2:1 ratio of the largest to smallest pores, an average pore diameter of about 50 Angstroms. As mentioned above, this diameter is an order of magnitude smaller than the smallest pore alluded to in the patent of Castro et al., and even when the top and bottom 15 per cent of the BET adsorption curve were neglected and in the definition of the S-valve of that document, an S value of 2 is approaching the limit of monodispersity in the disclosed material. It is also know that there is a need for improved biocompatibility in the encapsulating material (Sun (1987)), and from the point of view of all these criteria, the best encapsulating material can be formed by the polymerization of a cubic phase formed by a polymerizable analogue of a biological lipid such as those mentioned above, which would in many cases have natural pore diameters close to 50 Angstroms. Microencapsulation has also been suggested for use in other disorders requiring cell transplants, such as diseases of the liver, pituitary, and parathyroid.

### Separations Using Transport Proteins

Another exciting potential application of the fixation of proteins into cubic phases is in separations of high specificity, using transbilayer proteins which allow passage of only certain molecules, often against considerable concentration gradients. For example, the linear polypeptide antibiotic Gramicidin A allows small monovalent cations to cross a lipid bilayer, by forming channels (Chappell and Crofts (1965)). The fact that many biological functions rely on such proteins in controlling molecular transport points to some important potential medical applications for the present invention. The viability of taming such transport processes in vitro has been demonstrated recently in experiments in which synthetic bilayers were loaded with proteins isolated from cells, and functioning transport systems thus reconstructed. Included in this study were so-called band III proteins, which appear to play a fundamental role in the exchange of oxygen for carbon dioxide. Apparently the band III protein creates a transbilayer channel of just the right charge and size to pass Cl— and HCO3-. In the cell bilayer, many proteins have fairly high lateral diffusion rates; measurements of the lateral diffusion coefficient in the bilayer of rhodopsin, for example, indicate values of roughly 5×1−⁻¹³ m²/sec. Based on such figures it might seem that polymerization of the lipid, which will reduce the lateral diffusion rate by at least an order of magnitude, would interfere with the activity of the protein. However, many membrane proteins are actually restricted in their lateral mobility, at their active sites. Thus, rhodopsin has been incorporated into polymerized liposomes of 1, 2-bis (octadeca-2, 4-dienoyl)-sn-glycero-3-phosphocholine plus dioleoylphosphotidyl choline (DOPC), and shown to have retained its photochemical and enzymatic activity (Tyminski, Latimer, and O'Brien (1985)). The protein F₀F₁-ATPase from Rhodospirillum rubrum has been polymerized into synthetic vesicles, and interestingly its activity actually increased upon polymerization (Wagner, Dose, Koch, and Ringsdorf (1981)). Of course, this is not to say that all proteins retain their functionality upon fixation of the bilayer.

A wide variety of ions and small molecules are transferred across bilayers through transport proteins which open and close in response to specific ligand-binding, (ligand-gated channels) and others in response to changes in membrane potential (voltage-gated channels). These offer additional mechanisms by which the molecular transport could be regulated in the context of the present invention. Interestingly, the protein-free phospholipid bilayer is highly permeable to water but impermeable to ions (the permeability coefficient of Na⁺ across a lipid bilayer is on the order of 10⁻¹² cm/sec, for example). This could have implications of the present invention in the desalination of water, for example.

### As a Scientific Standard

The geometric precision and perfect lattice ordering of the present invention leads to important potential applications as a scientific standard, and, in fact, as mentioned above, there are now experiments under way in which the invention is being used as such. Certain areas of science and technology call for experiments in which there is need for precisely-controlled microenvironments on the length scale of the pores of this invention, and a few such areas are now discussed to illustrate the potential importance of this invention. Also discussed are the shortcomings, in many cases, of the material disclosed by Castro et al. and prior art microporous materials in such applications.

In the study of critical phenomena, it is know that fluctuations which have important effects on critical behavior can be induced by confining the system (fluid, fluid mixture, magnetic material, etc.) in a disordered porous material. There is a need in many cases to eliminate this source of fluctuations, and confine the system instead in a porous medium which has not disorder over a length scale greater than the correlation length of the system. In the superfluid helium experiments of Dr. John Reppy and coworkers at Cornell for example, the desire is to work close enough to the critical point that this correlation length is on the order of nearly a micron. The study of superfluids and super conducting fluids, and the phase transitions they exhibit, are an extremely active topic at present, and there is clearly tremendous potential in these systems. Another system of enormous potential technological benefit in which critical behavior appears to play a crucial role is in the use of microemulsions for tertiary petroleum recovery; it has been suggested that ultralow interfacial tensions (on the order of millidynes per cm.) between certain microemulsions and both oil and water are the result of near-critical behavior (Pouchelon, Chatenay, Langevin, Meunier (1982)).

In the study of fluids and fluid mixtures, it is known that the adsorption characteristics and phase transition temperatures are affected by porous materials. For example, there is an effect known as capillary condensation, in which the effect of pores is to cause thin films of condensate to develop on the pore walls. Obviously, in studies of such phenomena it is advantageous to eliminate pore size and shape as a variable. Recently it has been demonstrated theoretically, and in experiments on the heat of adsorption in zeolites, that the adsorption characteristics as well as the ability of porous media to crack hydrocarbons in zeolites of different structures were in remarkable agreement with the theory, which predicts a linear dependence of the heat on the average Gaussian curvature of the porous medium (Thomasson et al., Chem. 10:1056 (1987)). Experimental data on heats of adsorption of hydrocarbons in zeolites of different structures were in remarkable agreement with the theory, which predicts a linear dependence of the heat on the average Gaussian curvature over the surface of the zeolite porespace. This is then used to interpret the effectiveness of the zeolites in the cracking of petroleum. In the present invention, as in zeolites, the average Gaussian curvature can be precisely set by the pore size and geometry, and is of course uniform from unit cell to unit cell. The advantages, in many cases, of the present invention over zeolites have been discussed above.

### Choosing Pore Morphology and Size

As a note concerning pore shape, the applicant has demonstrated that transmission electron microscopy can be valuable in determining pore morphology in polymerized cubic phases. There are other experimental techniques which are useful in this respect; in particular, in recent years there have been many Scanning Electron Microscopy micrographs published, particularly of so-called ‘lipidic particles’, which are most likely cubic phases in actuality (Rilfors, Eriksson, Arvidson, and Lindblom (1986)). These SEM photos are obtained by fast-freezing the sample and then replicating the surface, although there have been serious criticisms of this technique as introducing artefacts. In addition, Luzzati and coworkers (Luzzati et al. (1988)) have recently developed a new technique of x-ray analysis which yield good-resolution electron density maps. The present application has shown (Anderson, D. M., Ph. D. Thesis, Univ. of Minnesota (1986)) how to compute candidate structures with interfacial surfaces of constant mean curvature, and predict the scattering intensities, for comparison with experiment, and shown that the method works well when applied to the DDAB cubic phase. These constant-mean-curvature structures were demonstrated, in the case of cubic phases in block copolymers, to be necessary for the correcting of this morphology based on both TEM data and thermodynamic calculations (above; also Anderson and Thomas, Macromolecules, in press).

In these determinations of pore shape and size, it is of prime importance that we are dealing here with an equilibrium morphology, and furthermore, a periodic morphology. In the nonequilibrium process of Castro et al., there is no hope that the pore shape could be determined to the same degree of accuracy. In fact, as stated on line 18 of page 17 of the Castro et al. patent, the manner in which the pores are formed is not even understood. A careful examination of the adsorption curves reveals that the size distribution of the pores, although much narrower than other microporous materials, is far from monodisperse: the most impressive of these curves is that in FIG. 30, and it can be seen that there is a significant volume of porespace tied up in pores of close to 0.8 micron diameter, as well as in pores of less than 0.2 microns (and since the latter pores are much less voluminous than the 0.8 micron pores, this means that their number density must be significant).

In many of the potential industrial, clinical, and research areas discussed herein, it will be of obvious advantage to extend the range of pore sizes in the present invention to the range of hundreds of Angstroms and even into the micron range. Above long-chained surfactants were discussed in this respect. For example, there are cubic phases in long-chained ethoxylated alcohol surfactants. for example, the surfactant C₇₀E₁₇—with a hydrocarbon chain of 70 carbons and 17 ethylene oxide groups—forms a cubic phase in water with a lattice parameter of approximately 500 Angstroms. This was determined by X-ray, which gives no direct information about bicontinuity. However, the ratio of hydrocarbon groups to ethylene oxide groups (or, equivalently, the hydrophile-lipophile balance or HLB) is approximately the same for this surfactant as for C₁₆E₄, which forms bicontinuous cubic phases (Mitchell, Tiddy, Waring, Bostock and McDonald (1983)). Both theory (D. M. Anderson and E. L. Thomas, Macromolecules, in press) and experiment (Alward et al., Macromolecules 19:215 (1986)) indicate that the lattice parameter scales as the 2/3 power of the molecular weigh, so that for example scaling the C₇₀E₁₇ surfactant to C₂₈₀E₆₈ can yield a cubic phase with a lattice parameter of approximately 0.125 micron. Indeed, lattice parameters well over 0.1 micron have been observed in block copolymers of polystyrene and polybutadiene (Hasegawa, Tanaka, Yamasaki and Hashimoto (1987)).

In addition, another means to produce cubic phases with very large lattice parameters—although in the metastable state—is to use very dilute surfactant concentrations. Lecithin is a component of certain cell bilayers (eggs and soybeans are common sources), and since the lattice parameters observed in prolamellar bodies and ER membranes are on the order of 0.1 micron or more, it is not surprising that these large lattice parameters can be created in vitro as well.

Another equilibrium microstructure which is very closely related to the cubic phase and often reaches characteristic length scales larger than 0.1 micron is the so-called “L3 phase” or “anomalous phase” (the French use the nomenclature “L2^{*} phase”). Work by the present author and coworkers (D. M. Anderson, H. Wennerstrom, and U. Olsson, J. Phys. Chems., submitted) has shown that the phase behavior, scattering, and NMR data on L3 phases can be explained by invoking microstructure for the L# phase which is essentially a disordered ( or “melted”) bicontinuous cubic phase. At low water contents, which are often attained in these L3 phases, the length scale of the microstructure can be greater than 0.1 micron even with short-chained surfactants. It is understandable that at such high dilutions, where the interactions between surfactant films become less important and therefore less of a stabilizing influence, that the structure should become more disordered, while still maintaining the basic topological characteristics of the ordered cubic phases. Thus in the C₁₆E₄/water system, for example, at approximately 40 percent surfactant and 70° C., the above-mentioned bicontinuous cubic phase appears, and is joined by a small two-phase region to an L3 phase region which extends to lower water contents. In related systems such as C₁₂E₅/water C₁₀E₄/water, this phase region extends to a few percent surfactant, and at these low concentrations length scales on the order of 0.1-0.3 micron are indicated both by a bluish visual appearance, and by rapid relaxation rates in NMR experiments (Nilsson and Lindman (1984)).

Specifically, our proposed microstructure for the L3 phase is locally a bilayer, which is highly-connected and topologically complicated as in the bicontinuous cubic phases but unlike the cubic phase is undergoing constant thermal disruption and thus does not posses long-range order. We then describe the bilayer by a base surface S, which is the mid-surface of the bilayer (the location of the ends of the hydrocarbon tails of the surfactant molecules), and the polar/apolar interface then consists of two parallel surfaces displaced a constant distance L on either side of S, where the length L is the bilayer half-thickness. By deriving the Euler-Lagrange equation for the curvature energy as a functional of the base surface S, it can be shown that S must tend toward a minimal surface (zero mean curvature) in order to minimize the curvature energy, registered at the polar/apolar interface. In binary bicontinuous cubic phases, it is now well-established that the base surface S is indeed a minimal surface, such as the so-called “Schwarz Diamond minimal surface” (Schwarz, H. A., Gesammelte mathematische Abhandlungen, Springer, Berlin, 2 vols. (1890)) or one of its relatives.

A key observation is that when the relation between the volume fraction of surfactant and the mean curvature at the polar/apolar interface is written, the properties of the minimal surface enter in a particular dimensionless number which is found to be nearly the same numerical value for all of the well-characterized minimal surfaces. This dimensionless number is the ratio of the third power of the surface area of a unit-edged unit cell to the Euler characteristic, multiplied by −2/pi. For all of the cubic-symmetry minimal surfaces with Euler characteristics less than 16 in magnitude for which the surface is known, this dimensionless number is within 8 percent of 2.2. Using the value 2.2, and assuming that the L3 phase can only occur when the mean curvature calculated from the resulting formula is equal to the “preferred” or “spontaneous” mean curvature dictated by the intermolecular forces between surfactant molecules, yields accurate predictions for the positions of the L3 phase regions over a range of surfactant/water systems. Thus, by virtue of the apparent universality of this dimensionless number, many of the properties of the L3 phase can be estimated without a more detailed knowledge of the exact microstructure. It can then be shown that the length scale, or “pseudo-lattice parameter”, of the microstructure varies inversely with the surfactant volume fraction (this pseudo-lattice parameter is defined as the edge-length of a cube which, on the average, enclosed a surfactant film with Euler characteristic of approximately −4). In the present context this is a key result, in that very large pseudo-lattice parameters can be found at very low surfactant concentrations, and our analysis indicates that even with short-chained surfactants such as C₁₀E₄, characteristic lengths on the order of 0.2 microns can easily be attained.

The theory also has the power to predict the location of cubic and L3 phase regions in phase diagrams based on molecular parameters of the surfactant. Using equation (47) of a paper by Cantor (R. Cantor, Macromolecules, 14:1186 (1981)), the degree of water penetration into the head group region of the surfactant bilayer can be estimated from a knowledge of the Flory-Huggins interaction parameter between the polar moiety and water. For ethylene oxide head groups, for example, this interaction parameter is known from experiments by Kjellander and Florin (1981). values for the number of water molecules per ethylene oxide (EO) group penetrating into the EO region of the surfactant film computed with the Cantor formula, using this interaction parameter, agree well with values estimated from NMR experiments. The theory of Cantor also predicts the dependence of the spontaneous (or “preferred”) means curvature on temperature, which can be linearized to a very good approximation. These equations are then combined with the equation described above linking the volume fraction in the bilayer (including the water penetration), phi_{B}, with the mean curvature H at the polar/apolar interface, namely phi_{B}²=−2.2HL (the minus sign is the convention for curvature toward water), to solve for the curve in the surfactant/water phase diagram along which the spontaneous mean curvature of the interface is exactly satisfied by a cubic phase geometry, or approximately satisfied for a disordered L3 phase geometry. The calculated curves agree well with experimentally observed L3 phase regions in ethoxylated alcohol surfactant systems. The theory also gives the correct shape of the L3 shape regions in phosphoryl surfactant and glycerol surfactant systems, although the lack of data on the interaction parameters for these polar groups precludes the possibility of a quantitative fit. And the theory provides a very good fit of the L3 phase region in a ternary system, C₁₂E₅/tetradecane/water.

This theory is thus a significant extension of the results of earlier work by the present author (D. M. Anderson, S. Gruner, and S. Leibler, Proc. Nat. Acad. Sci., in press), in which the variances in means curvature and bilayer width were computed for model cubic phase structures, showing conditions under which the cubic phase should be expected to most closely satisfy the curvature tendencies of the interface. Together they provide a means to predict, to some extent, temperatures and compositions at which cubic phases or L3 phases would be likely to exist. The theory of Cates et al. (Cates, Roux, Andelman, Milner and Safran (1988)) represents another attempt to interpret the location of L3 phases, but it suffers from two serious flaws:

1. the entopic contributions to the free energy for the L3 and lamellar phases, which are central in the theory, are computed by entirely different means in the two cases, and thus the comparison is not very meaningful; and

2. it is assumed in that paper that the spontaneous mean curvature of the interface is zero, whereas the present author has shown (D. M. Anderson, H. T. Davis, and L. E. Scriven, J. Chem. Phys., submitted) that in fact the mean curvature of the interface in their model is toward the solvent (e.g., water). On the contrary, in our theory, simple mathematical arguments show that a bicontinuous structure is a simple consequence of spontaneous mean curvature toward water in a bilayer structure and it is demonstrated that the locations of L3 phases in surfactant/water phase diagrams strongly indicate spontaneous curvature toward water.

If indeed it is true that L3 phases are bicontinuous, then they provide another means to produce microporous materials in the manner of the present invention, and a polymerized L3 phase would possess many of the favorable and novel features of a polymerized cubic phase with the exception of triple-periodicity. A primary technical complication in the actual production of such a material would be the fact that as in microemulsions, the structure is thermally roiled and undergoing continual rearrangement on microsecond timescales, so that the structure could easily rearrange significantly during the polymerization process; recall that, as noted above, a recent attempt to polymerize a bicontinuous microemulsion resulted in a loss of bicontinuity (Candau, Zekhnini and Durandi (1988)).

### Affinity Based Separation

In the study of proteins, the potential importance of the present invention is clear from all that has been said here. Precise control of the environment of the protein to be studied, chemical steric, and electrostatic, uniformly over the entire sample cannot be overestimated. One more word can be said, however, and that concerns an important laboratory technique—which also has potential technological and clinical applications—that is known as affinity-based separation. In this process, the target biomolecule to be separated from solution attaches to a ligand with specificity toward the target molecule. the ligand+target is then separated from the other proteins in the solution by ultrafiltration, and the target and ligand are then dissociated and ultrafiltration is used again to separate these. Presently the use of this technique is limited by the fact that a ligand must be chosen which is much larger than the target molecule: the rule of thumb presently is that the ligand should be at least 10 times larger than the target, due to the polydispersity of present ultrafiltration membranes. Clearly the present invention has the potential to drastically reduce this requirement and to permit simpler, more efficient, and more available separations for biomolecules, for subsequent study in the lab, or application in industry or medicine.

### Creating Asymmetry

For many of these potential applications, it will be necessary to create an asymmetry between the two labyrinths—chemical, electrical, or geometrical —in order to effect a separation between reactants, reaction products, catalysts, or filtrates. At present, the precise mechanism is not known by which this asymmetry is created in living cells. Nevertheless,the very nature of the bioprocesses, such as photosynthesis, which rely on this asymmetry prove that chemical asymmetry is indeed created, and in the case of the thylakoid membrane and the prolamellar body there electron microscopy data which demonstrate geometrical asymmetry. For example, measurements made from micrographs or prolamellar bodies—which are known to have cubic symmetry —indicate that the surface areas of the two head group surfaces differ by approximately 30% (Israelachvili and Wolfe (1980)). It is possible to mimic this mechanism to create the desired asymmetry within the context of the present invention, namely through the use of polymerizable surfactants. There are already several possible means by which asymmetry between the two labyrinths can be created:

1. As mentioned above, in the most common cubic phase microstructure, of Ia3d space group, the two labyrinths are of opposite chirality, and it has recently been shown that a chiral protein, cytochrome, locates solely in one labyrinth and not in the other (Luzzati, Mariana, and Delacroix (1987)). This asymmetry should change the space group of the structure and indeed a change in space group was observed. This demonstrates the feasibility of creating asymmetry through chirality effects. Furthermore, it could in fact lead directly to microporous polymeric material with the ability to separate enantiomers, because the polymerization of the surfactant in such a structure would leave only one labyrinth, exhibiting a chiral porespace. Presently, the separation of enantiomers is generally a very expensive and inefficient process in the chemical industry and in research, and the availability of such a filter is a major advance made easier by the present invention. The material disclosed in Castro is not suited for such applications.

2. Recently, epitaxial relationships have been demonstrated between bicontinuous cubic phases and hexagonal lamellar phases (Klason 1984; Rancon and Charvolin (1988)). In the binary C₁₂E₆ systems, in which monodomain cubic phases can be grown with very little effort, it has been shown in two research groups that upon lowering the temperature from the cubic phase region to the hexagonal phase region, the hexagonal phase microcrystallites grow in a precise epitaxial relationship to the cubic phase. Specifically, the cubic phase is of the Ia3d type discussed in the previous paragraph, and the cylinders of the hexagonal phase grow along and directions given by the ‘tunnels’ of the cubic phase. If such a system is polymerized, this creates accesses to the two labyrinths of the cubic phase through two distinct systems of hexagonal phase cylinders distinguished by their orientations. This would be in close analogy with the microstructure in the endoplasmic reticulum, in which the smooth ER is a finely porous network, observed in some electron micrographs to possess cubic symmetry (Albers, Bray, Lewis, Raff, Roberts, and Watson (1983)), that connects to the rough ER of much coarser structure and simpler topology. Examples of epitaxial relationships between cubic phases and other liquid crystalline phases have been observed in electron micrographs of bicontinuous cubic phases which are apparently involved in digestion, and this has lead to a variety of speculations about the role of cubic phases in digestion (Luzzati (1987)).

3. Even though the mechanism leading to asymmetry in vivo is not yet understood, it can be reproduced, by substituting polymerizable phospholipids into extracts from biological cubic phase systems. The feasibility of such a scheme is demonstrated by experiments in which liposomes produced from phosphotidyl choline have been fused to broken thylakoid membranes (Tien (1981)). In addition, lipids extracted from prolamellar bodies have been shown to aggregate into branched tubular structures similar to the (asymmetric) in vivo bicontinuous cubic phases of the prolamellar body (Kesselmeier and Budzikiewicz (1979)). This scheme could open up some extremely exciting possibilities in capturing the basic processes of the cell for study or for the synthesis of biological compounds, or the harnessing of photosynthesis, for example.

Other methods are available for obtaining large cubic phase domains and/or domains of a desired orientation. It is well-known that electric or magnetic fields can be used to orient liquid crystals. For example, the C₁₂E₆/water cubic phase was observed to orient in the magnetic field of an NMR spectrometer in experiments of Klason (1984); upon lowering of the temperature into the hexagonal phase region, the hexagonal phase microcrystallites were all in one of four tetrahedrally-related orientations, bearing a precise relation with the applied magnetic field. This latter observation points to another possible means, namely that cubic phases of large, oriented domains could be obtained by cooling or heating an oriented lamellar or hexagonal phase—and it is well-known that the latter phases are rather easily aligned by shear and by the effect of walls. In addition, temperature cycling is also an effective method for increasing crystallite size in cubic phases. This could be related to the observation (Shyamsunder, Gruner, Tate, Turner and So (1988)) that cubic phases in dioleoylphosphotidylethanolamine (DOPE) can be induced by temperature cycling.

In reaction involving charged species, the reaction products, confined to the two separate labyrinths, could be routed in opposite directions through the use of an imposed electric or magnetic field. A related possibility would be to take advantage of the opposite chiralities of the two labyrinths in the Ia3d cubic phase by imposing a rotational electric or magnetic field which would induce opposite net flows in the left- and right-handed screw networks.

### Microdevices and Molecular Electronics

As mentioned above, the triple-periodicity of the present invention combined with the small length scale attainable—considerably less than 0.1 micron—brings up potential applications in metal and semiconductor microstructures, and indeed the frontiers of microfabrication are now moving into the range of molecular dimension where this microporous device provides the only triply-periodic microenvironment available, except for zeolites which are limited to 2 nanometers or less. At these length scales, quantum effects become pronounced and in such a medium with extremely high surface-to-volume ratios properties are often dominated by the surface condition. According to M. J. Kelly (1986: “The physics of fabricated microstructures represents the current frontier of condensed matter physics . . . . Once two or more of the length dimensions of a structure are 0.1 micron or smaller, the mode of operation of any device becomes qualitatively different from that of the larger devices in current use. . . . The ability to tailor three-dimensional nanometer scale structures in a wide range of materials may lead to synthetic solids with more desirable device properties than those provided by nature. . . . ”

The potential importance of surfactant microstructures in quantum-based devices has been shown in experiments on polymerized Langmuir-Blodgett films (Larkins, Thompson, Ortiz, Burkhart and Lando (1983)). These workers demonstrated superconductivity and Josephson effects at 4.2K in polymerized LB films of vinyl stearate and diacetylene. As discussed by Roberts (1985), this indicates potential applications in the control of the critical current, switching speed and energy gap parameters in low temperature devices. Roberts also discusses possible applications of magnetically ordered polymerized LB films as switches in superconducting junctions.

Molecular electronics is predicted by some to be emerging within the next few decades, and surfactant microstructures have been discussed as providing potential memory and switching devices because they involve a great deal of self assembly, and also because electro-optical and photochromic effects are higher in organic than in inorganic materials. For example, polymerizable conjugated diacetylene surfactants become intensely colored upon polymerization (for example, by UV light), and electronic memories based on such photochromic effects have been speculated (Wilson 1983). Also, primary pyroelectricity has been reported in LB films (Blinov, Mikhnev, Sokolova and Yudin 1983), and this has led to speculations concerning possible incorporation of IR-sensitive surfactant films into electronic devices for imaging or sensor applications. The non-centrosymmetricity of X and Z type LB films can give rise to optoelectrical effects, and in this respect it is of potential importance that the cubic phase incorporating cytochrome c, discussed above, possesses a non-centrosymmetric space group. One should also note that cytochrome c is a colored protein which acts as an electron carrier in the electron-transport chain of cell.

While such applications are highly speculative at this point in time, they have lead to a great deal of research recently on LB films, monomeric and polymerized, at low temperatures, with metal ions or enzymes incorporated, in non-centrosymmetric configurations and between semiconductors and metal electrodes, for some examples. For some of these potential applications, the polymerized cubic phase of the present invention could be important in providing a periodic, three-dimensional microstructure with a very high surface area and a single continuous surfactant film, together with enhanced quantum effects due to confinement in nanometer-sized pore bodies.

### Further Experimental Results and Projections

1. Cross-linked Cubic Phases:

We have produced cross-linked polymerized cubic phases, which we intend to characterize by scanning electron microscopy, after drying by supercritical drying. SEM offers several advantages to TEM in this respect: first, since microtoming will not be necessary, there will be less disturbance to the sample during preparation for the microscopy; and second, this will give direct information concerning the structure of the material at the macroscopic surface, which is all-important in determining flow properties. The particular cubic phase we have prepared for this experiment is a DDAB/styrene+cross-linker/water cubic phase, which has very good physical integrity and which undergo a glass-rubber transition during the supercritical drying (as would PMMA, for example). The mechanical integrity of the final material was very good; it is at the bottom of a vial, and ethanol can be used to fill the vial and the vial can be shaken without apparent disturbance of the material.

2. Sieving Particles:

Two membranes can be prepared by the polymerization of two cubic phases at slightly different compositions, and we can sieve particles or macromolecules of a narrow and precise size fraction. The DDAB/styrene+cross-linker/water cubic phase exhibits an increase in lattice parameter of approximately 3 Angstroms per percentile of water, so that the pore sizes in the two membranes can be chosen to be, say 90 to 110 Angstroms. A solution containing microspheres of several sizes, say 100 and 125 Angstroms diameter, will be passed first through the 110 Angstroms membrane, and the filtrate then passed through the 90 Angstroms membrane, so that the 125 Angstroms spheres should be rejected by the first filter and the 100 Angstroms spheres by the second. Similarly, a mixture of a wide MW range of polymers or proteins can be passed through the two filters sequentially and the fraction rejected by the second filtration can be checked for polydispersity index by standard techniques.

3. Near-critical Behavior:

As mentioned above, the group of John Reppy at the University of Cornell has indicated that they will have a BET adsorption isotherm done on the specimen that we have provided them. This will then be tested as a highly-ordered microporous material in experiments on the near-critical behavior of superfluid 4He.

4. Single-crystal:

The C₁₂E₆ cubic phase can be polymerized to obtain a monodomain (or “single crystal”) specimen. This can be then characterized by single-crystal X-ray techniques; the orientation of the lattice would be known from the preparation. This would be an aqueous-phase polymerization, because the aqueous phase is a single labyrinth whereas the surfactant is divided into two, disjoint continuous networks. We have been able to incorporate 25 percent monomeric acrylamide into the aqueous phase.

5. Enzyme Incorporation:

Using a polymerizable surfactant, an enzyme such as glucose oxidase can be incorporated into a cubic phase, smeared onto the tip of a pH meter probe, and fixed by polymerization. The probe is then dipped into a glucose solution and the pH measured as a function of time. A drop in the pH would indicate the oxidation of glucose by the immobilized enzyme.

6. Cytochrome-c Incorporation:

We can incorporate cytochrome c into a cubic phase as in the experiments of Luzzati and coworkers, except with the polymerizable analogue of monoolein. After polymerization, racemic mixtures of different compounds would be passed through the membrane, and the filtrate tested for optical activity. It is not expected that every sized molecule can be separated by chirility in this manner, but for molecules with sizes slightly smaller than the pore size, the separation of enantiomers should be possible in many cases, with the separation increasing with the number of passes through the membrane.

7. High Organic Concentration:

Samples are now being prepared of the type described by Scartazzini and Luisi for SAXS analysis, to determine if indeed they are cubic phases. Since these occur at very high concentrations of organic and very low concentrations of water, they would open up many interesting systems in composition regimes which are relatively unexplored.

8. Large Lattice Parameters:

The cubic phases of very large lattice parameters investigated by Helfrich and coworkers can be investigated for possible polymerization and characterization. In this case the characterization should be made much more straightforward because these structures are visible in the optical microscope.

9. Photocatalysis:

We can perform the photocatalytic experiments described by Willner et al. but in polymerized bicontinuous cubic phases, in which the surfactant is the polymerized species. The particular surfactant used can be a quaternary ammonium surfactant similar to DDAB but with two double bonds in each tail (so four polymerizable sites per molecule). We can prepare a cubic phase very similar in composition to the DDAB/decano/water cubic phase examined in the author's thesis (but with toluene replacing decane), because this is a ternary cubic phase with a monolayer of surfactant dividing oleic and aqueous labyrinths, and the oleic regions are necessary in the system used in the Willner et al. experiments.

10. Ionic Pore Walls:

A cubic phase can be formed with styrene, water, and a polymerizable analogue of DDAB first of all because there are many different polymericable quaternary ammonium surfactants in the literature, and second of all because DDAB is a very persistent cubic-phase former, as evidenced by the large cubic phase regions in many ternary DDAB/water/oil phase diagrams, then we can polymerize both the styrene AND the surfactant, so to create a microporous material with ionic porewalls.

11. We will continue to take the DDAB/styrene/water cubic phase to higher temperatures, and at the upper stability limit, perform a thermally-initiated polymerization reaction of a sample of large volume.

12. Acrylamide:

Acrylamide has been added to the water component of

a) the DDBA/water/dodecane cubic phase, and

b) the C₁₂E₆/water cubic phase

13. Enzyme Immobilized in a Lipid-water Cubic Phase:

Proteins can be incorporated, in fairly high concentrations, into bicontinuous cubic phases made with polymerizable lipids that are biocompatible. Glycerol monooleate, or -monoolein, is an uncharged biocompatible lipid (e.g., present in sunflower oil), with one fatty acid chain containing a single double bond. A variant of monoolein with a conjugated diene in the chain is monolinolein, and the monolinolein-water phase diagram is known to be nearly identical with that of monoolein-water. As discussed above, the #212 cubic phase structure has been found in the [monoolein/water/cytochrome-c] system, and the present authors have found the same structure at 6.7 wt % cytochrome, 14.8% water, and 78.5% monolinolein, where the monolinolein contains 0.4% AIBN. After equilibration, this cubic phase was placed in the UV photochemical reactor in a water-jacketed cell and bathed in nitrogen in the usual manner. After 48 hours the sample had polymerized and could be held by a tweezers, and was a deep red color, as in the unpolymerized phase, due to the strongly-colored protein. X-ray of the polymerized sample appeared to be consistent with space group #212, with a lattice parameter of approximately 110 Angstroms, although the Bragg reflections were very weak.

14. Polymerization in a Nonionic System:

Polymerization of the bicontinuous cubic phase in the system [didecy hexaethyleneoxide (C₁₂E₆ water] has also been performed using acrylamide as the aqueous monomer, and the polymerized phase shown by X-ray to have retained its cubic ordering. The acrylamide made up 19.96 wt % of the aqueous phase, and hydrogen peroxide was used as the initiator at 1.1 wt % of the acrylamide. This aqueous phase formed 30.30 wt % of the total mixture. The polymerization was performed in a nitrogen atmosphere at 23° C., via UV irradiation. Following polymerization, the phase was soaked in ethanol for several weeks, to replace all components except the polymer gel. An X-ray analysis was then performed on the polymerized sample, and indexing of the resulting powder pattern revealed a cubic structure of space group #230, with a lattice parameter of 93 Angstroms. At 38 wt % water, 62% C₁₂E₆, Rancon and Charvolin reported the same space group in an unpolymerized phase, with a lattice parameter of 118 Angstroms. In contrast to the latter experiments, no steps were taken to produce a single crystal sample; however, in view of the fact that monodomain cubic phases are relatively easy to produce in this system, a monodomain polymerized cubic phase, exhibiting single crystal texture in X-ray analysis, can be produced.

The successful polymerization of this cubic phase is also of potential importance in that, by keeping the ratio of ethylene oxide to hydrocarbon groups fixed and increasing the molecular weight of the surfactant, it is possible to produce polymerized bicontinuous cubic phases with a continuum of pore diameters up toward the micron range.

In particular, indexing of X-ray patterns from seven [CₙEₘ/water] cubic phases, with n-17 and m-70 (surfactant mixture obtained from Berol, Inc.) between 25 and 55% surfactant, is consistent with the bicontinuous #230 structured discussed above (data courtesy of K. Fontell). The conclusion that these cubic phases made with high molecular weight surfactants are indeed bicontinuous was also demonstrated by NMR self-diffusion measurements. Self-diffusion measurements were performed using the Fourier transform pulsed-gradient spin-echo (FTPGSE) technique, with 1_{H} NMR, on a modified JEOL FX-60 NMR spectrometer, operating at 60 MHz. The method as practiced at the University of Lund has been described in detail in: U. Olsson et al., J. Phys. Chem. 90:a4083-4088 (1986). The self-diffusion constant for the aqueous component (HDO, present in trace amounts in D₂O), after suitable corrections for hydration of the ethylene oxide groups, was 4.0×10⁻¹⁰m²/sec. The surfactant self-diffusion constant was 2.5×10⁻¹⁰m²/sec. For comparison, at much lower molecular weight there exist two cubic phases in the C₁₂E₈/water system, one of which is bicontinuous and one of which is not (the latter is made up of discrete micelles). In the bicontinuous phase case (which has the Ia3d, #230 structure), the surfactant self-diffusion has been found to be 8×10⁻¹⁰m²/sec (Nilsson, Wennerstrom, and Lindman (1983)), whereas in the discrete cubic phase the surfactant self-diffusion rate in the high-MW case is actually higher than that in the low-MW discrete case, and only a factor of three lower than that in the known low-mW cubic phase; the factor of three is of course due to the slower diffusion associated with a higher-MW molecule (larger by about a factor of about six). The high diffusion value for the water component then also demonstrates water continuity, which is not surprising because the sample is high in water content. Thus the X-ray results, indicating a bicontinuous structure, are confirmed by this self-diffusion experiment. These experiments prove that bicontinuous cubic phases exist in high-MW surfactant/water systems, and in fact, as the MW gets higher in these systems, the composition range over which the bicontinuous cubic phase exists get very wide. In this case, it exists from 25 to 55% surfactant at room temperature.

15. Thermoposimetry:

Thermoporometry was used to characterize the pore size distribution of a polymerized cubic phase. This measurement is based on the principle that the melting (and freezing) temperature of water (or any fluid) is dependent on the curvature of the solid-liquid interface, which depends on the size of the pore in which the interface is located. For the melting of ice into water inside a cylindrical pore of radius R (in nanometers), the melting temperature is decreased by an amount of T (in degrees Celsius) given by [Brun 1977]:

T=32.33/(R−0.68) for melting, and

T=64.67/(R−0.57) for freezing.

For a pore with radius R=100 Angstroms, for example, this would be a drop in melting temperature of about 3.47° C., which is easily detectable with a differential scanning calorimeter (DESC). The method applies for pores between 20 and 200 Angstroms in radius. Only in the case of a microporous material with very monodisperse pores does the resulting DSC scan exhibit a peak at this offset temperature, with a return to the baseline before the second peak at 0° C. arising from bulk water around the sample.

The primary advantages of thermoporemetry over other porosimetry methods, such as BET porosimetry, are 1) it is a simple, straightforward measurement made with standard equipment, and 2) the sample does not need to be dried, and thus supercritical drying need not be performed. Thus, the material is investigated under conditions which are most similar to those conditions encountered in normal use.

The cubic phase examined with thermoporometry was a monolinolein/water/cytochrome-c cubic phase prepared according to the method of Mariani, Luzzati, and Delacroix (1988); their preparation used monoolein instead). The resulting sample was in the two-phase region at 23° C., which is an equilibrium between two bicontinuous cubic phases, one with space group #212 and the other, at higher water content, with space group #229. Therefore, the exact composition of the same was not known. However, those authors performed X-rays on four samples in these two regions of the phase diagram and their estimates of the radii of the aqueous channels were in all four cases within 4 Angstroms of R=16.7 Angstroms. Our monolinolein sample contained AIBN as initiator, and was exposed to UV radiation for 48 hours. The polymerization of this lipid has been inconsistent. In some cases, complete polymerization results and the sample is quite solid, while in other cases, several days of exposure does not bring about complete polymerization. The reason for this is as yet unknown, but the elimination of oxygen from the sample seems to be the most difficult step. A partially polymerized sample was examined with thermoporometry. This sample was chosen for the experiment because this cubic phase structure provides the most nearly cylindrical pores upon polymerization, and the equations of Brun are derived under the assumption of cylindrical pores. In more complicated pore shapes, the relationship between the pore size and shapes, the relationship between the pore size and shape, and the mean curvature of the solid/liquid interface, is more complicated.

About 16.5 mg of the specimen was then examined in a Perkin Elmer differential scanning calorimeter, model 1 DSC II¹. On the freezing scan, the freezing began at about 222° K. and the Brun equation yields a pore radius of R=18.4 Angstroms. The maximum corresponds to R=17.7 Angstroms. The melting curve shows more complicated behavior above 240° K. (part of which is due to the melting of free water at 273° K.), which we do not fully understand yet. Since there is a hydrated protein present, some of the melting at high temperatures (=266° K.) is probably due to the water hydrating the protein. Nonetheless, focusing on the hump near 236° K., we again see evidence for monodisperse water-filled pores. The hump starts at about 230° K., which corresponds to about 16.3A. Putting all of this together, we see that the thermoporemetry gives good evidence of monodisperse water-filled pores with radii of approximately 14 to 18 Angstroms, which is in excellent agreement with the radius expected from the X-ray results of Luzzati and coworkers. ¹ See FIGS. 8 and 9.

16. Immobilization of Glucose Oxidase:

The enzyme glucose oxidase was incorporated into the aqueous phase of a cubic phase and this aqueous phase polymerized by the addition of monomeric acrylamide. Except for a slight yellowish color from the strongly colored glucose oxidase, the result was an optically clear polymerized material. The concentration of enzyme in the aqueous phase was 10.3 mg/ml, the acrylamide concentration was 15.4 wt %, and hydrogen peroxide as initiator was present at 0.3 w/w % of the monomer. This aqueous solution was mixed in a nitrogen atmosphere with 24.3 wt % DDAB and 10.93 wt % decane, and the solution centrifuged for one hour to remove any remaining oxygen. This water content, 64.8%, was chosen based on SAXS study of the cubic phase as a function of water content in similar systems. Above about 63 vol % water, the lattice parameter is larger than 175 Angstroms with either decane or decanol, the aqueous regions should be large enough to contain the enzyme.

Two samples were prepared for polymerization. One sample was simply placed in a quartz tube and polymerized for X-ray analysis. The other was smeared onto a nylon backing which had been shaped to fit on the end of a pH probe. Both samples were bathed in nitrogen during UV irradiation. The first sample was about 1.5 mm thick and after polymerization was a clear solid which could easily be handled; this was loaded into a flat SAXS cell with mica windows. Indexing of the resulting peaks to a BCC lattice indicated a lattice parameter of 320 Angstroms. The second polymerized sample was soaked for one day in ethanol to remove the DDAB and decane, and then secured over the tip of a pH probe, and the enzyme was shown by the method of Nilsson et al. (37) to have retained its activity in the polymerized cubic phase. This example is a demonstration of a general application, namely in biosensors. In many cases the substrates to be detected are of a higher molecular weight than glucose and the porespace created by the cubic phase microstructure can be tailored to the size of the substrate.

### Further Details of Materials Incorporating Bio-active Agent

There is an additional advantage of this material over other materials in the physical entrapment method. This is the fact that the pore size, which is determined by the cubic phase microstructure, can be preselected independently of the mesh size of the cross-linked polymer network. Consider the usual method of entrapment, in which a cross-linked polyacrylamide gel is used to entrap the enzyme. In such a case the polymer concentration and the extent concentration and the extent of cross-linking must be such that the mesh size of the gel is a) small enough to entrap the enzyme with a minimum of leakage; but b) large enough to allow flow of the substrate and product(s) in and out of the gel; and c) optimal in terms of the mechanical properties of the gel. Often these are competing requirements and comprises must be made. However with the cubic phase material, the access of the substrate to the enzyme is through the (periodic) pore system created by the cubic phase, and this can be adjusted independently of the concentration of polymer and cross-linker in the aqueous phase.

For example, the DDAB/decane/water+acrylamide+cross-liner system (where the cross-linker is for example N,N′-methylenebisacrylamide), the pores created by the cubic phase microstructure result from the removal of the unpolymerized components, DDAB and decane, and the diameter of these pores can be varied between 60 and 150A by varying the total concentration of water+acrylamide+cross-linker between 35 and 65%. Independently of this, the relative concentrations of acrylamide, water, and cross-linker can be varied so as to adjust the final properties and entrapping ability of the polymer gel.

This property could be made good use of in the case of high-molecular weight substrates, which until now have been very difficult to handle with immobilized enzymes. If one simply entraps the enzyme in a PAM gel with access of the substrate only through and polymer mesh, then this mesh size must be made very large for high-MW substrates, and this means a dilute polymer concentration and low cross-linking and therefore very poor mechanical properties. However, with the present invention one can still have a high polymer concentration and cross-linking because access to the enzyme can be through the porespace created by the cubic phase microstructure, and these pores can be made to have diameters of over 100 Å.]]

There are several other general means by which the present material can be used in the immobilization of enzymes, or biocatalysts in general, besides entrapping the biocatalysts in the polymerized component. In fact, this material is potentially of use in all of the presently-used for immobilization. Besides physical entrapment, which has already been discussed and shown to be feasible, we now consider alternative methods of immobilization and the advantages that could be provided by the present material over and above the advantages traditionally associated with each method.

### Covalent Bonding and Adsorption of Enzymes

When most people hear the term “Immobilized Enzyme”, they think of enzymes which ar covalently bonded to an insoluble support, which is usually polymeric. In the present invention, enzymes can be covalently bonded to the porewall surface of the polymerized cubic phase, thereby inheriting the precision, biocompatibility, and versatility of the invention together with the usual advantages associated with covalently bonded enzymes. These advantages include permanence of the immobilization, so that the product is not contaminated with the enzyme and the enzyme is not lost due to changes in pH, temperature, etc., as in adsorbed enzymes. Also, in some case (though certainly not all) a covalently bonded enzyme exhibits enhanced chemical or physical characteristics over the soluble enzyme, due to the alteration in its actual chemical structure. Furthermore there is a high degree of development in this form of immobilization, so that a wide variety of support polymers can be used and years of experience can be drawn on.

Covalent bonding or adsorption of a biocatalyst to the porewall surface of a polymerized cubic phase would create a reaction medium in which the pore size would be selected so as to allow access to the enzyme only for selected components. This would be of considerable importance in cases where the substrate was not isolated in a simple solution but rather present together with many other components, some of which could be detrimental to the desired reaction. Clearly one important example would be blood, in which immunoglobulins, blood cells, and various macromolecules could be selectively excluded from enzyme contact by the monodisperse pores. In the more general case, it should be possible in many cases to use size exclusion to eliminate inhibitors (such as protein inhibitors) from the site of reaction while still allowing access of the substrate to the biocatalyst.

Several methods have been discussed (high-MW nonionic surfactants, dilute lecithin concentrations, etc.) for producing polymerized bicontinuous cubic phases with very large pore sizes, and the covalent bonding or adsorption of a biocatalyst to the porewall surface of such a material would open up the possibility of reactions with high molecular weigh substrates in highly controlled membrane materials. Enzymes covalently bonded to polymeric particles suffer from the unavoidable steric repulsion of high-MW substrates, so that these substrates have traditionally been difficult to handle with the usual immobilization schemes. However, with the present invention in membrane form, high-MW substrates could be forced through the porespace with pressure as in any ultrafiltration process, and the high porosity and pore uniformity would allow this flow to be established with the minimum possible pressure. With the wide poresize distributions which characterize prior art isotropic membranes, the pressure needed is determined by the smallest pores, and these may be much smaller than the nominal pore size. And hollow fiber bundles or capillary array filters cannot achieve the high porosity, high specific surface area (over 3,000 m²/gm in some cases), and resistance to clogging that are made possible by the highly-interconnected porespace of the present material.

We have formed polymerized bicontinuous cubic phases in which the polymeric matrix is a polyacrylamide (PAM) gel, and it is well-known that PAM is chemically stable, resistant to hydrolysis in the pH range 1-10, does not react with nitrous acid, etc. However, PAM can be modified chemically and subsequently coupled to an enzyme covalently, and in fact this is the most widely used polymer for the covalent bonding of enzymes. Beads of PAM gel are commonly used to covalently bond enzymes, but with beads specific surface areas are on the order of at most tens of square meters per gram, whereas the present material offers hundreds or even thousands of square meters per gram. Furthermore, initiators for the polymerization of acrylamide can be found in biological sources, such as riboflavin.

In many cases it would be advantageous to have the biocatalyst immobilized in a dispersion or suspension of particles, such as when the preparation is to be injected into the body or absorbed through the skin, for example, or to make the enzyme more accessible to the substrate through simple diffusion. There are many possible methods which could be used to produce dispersions of polymerized cubic phase particles, including the following:

a) Winsor and Gray (1974) have described an experiment in which relatively mondisperse, polyhedral-shaped particles of (unpolymerized) bicontinuous cubic phase spontaneously formed and were photographed through an optical microscope. An aqueous preparation of the anionic surfactant “Aerosol TO” was dried in the microscope and when the concentration reached that corresponding to the well-known bicontinuous cubic phase between 78% and 84% AOT (Fontell 1973), polyhedral particles of approximate diameter 10 microns were observed to form. Photographs of these particles were published in the Winsor and Gray volume. At present we are at work to reproduce this experiment with AOT and hopefully, other surfactants and lipids, and eventually to polymerize such particles.

In addition to AOT, glycerol monooleate (monoolein) has been shown to form polyhedral microcrystallites of bicontinuous cubic phase (M. Linstrom, H. Ljusberg-Wahren, K. Larsson and B. Borgstrom 1981). Furthermore, a small amount of sodium cholate can be used to obtain a dispersion which is quite stable. Conjugated bile salts can also be used to disperse the particles. It should also be mentioned that the cubic phase made from sunflower oil monoglycerides and water can incorporate hydrocarbons, at least up to 5:95 weight ratio of hexadecane to monoglycerides, and in principle then also polymerizable hydrophones. Sunflower oil monoglycerides are available for a remarkably inexpensive price: approximately 25 SEK per kilogram.

There exist many ways in which phospholipids can be induced to form bicontinuous cubic phases. We have already discussed the temperature cycling experiments of Gruner, in which a cubic phase was induced by cycling above and below the lamellar/inverted hexagonal phase transition temperature many hundreds of times. Other work by Gruner has shown that small modifications in the polar head group of phospholipids can lead to cubic phase-forming phospholipids. This is primarily a curvature effect, and similarly modifications of the fatty acid chains could be used to create the same result. But another way is the use of mixtures of lipids. To give three representatives examples: 1) monoolein can be added to the DOPC (dioleoylphosphotidyl choline)/water system to induce a bicontinuous cubic phase; 2) sodium cholate can be added to the lecithin/water system, and a cubic phase results in approximately the center of the ternary phase diagram; and 3) although MGDG and DGDG do not form cubic phases in their respective binary phase diagrams, there is a cubic phase in the ternary MGDG/DGDG/water phase diagram.

b) We have produced a dispersion of polymerized bicontinuous cubic phase particles, with estimated sizes of 1 to 10 microns. The starting material was actually the result of what was thought to be an “unsuccessful” experiment. The DDAB/water/styrene cubic phase discussed at length above was prepared using less than 75 styrene and no cross-linking agent. Under these conditions it is not surprising that after polymerization, the polymer could easily be broken up by mechanical disruption, and in fact after 30 minutes of sonication, a very fine dispersion of particles resulted. This sonication was performed after replacing the unpolymerized components with methanol, and sedimentation was then avoided by adding approximately 1.7 parts of 2-chlor-ethanol per 1 part of methanol, in order to match the gravimetric density of the fluid to that of the (microporous) polystyrene particles. The dispersion was white in transmitted light and slightly bluish, and some particles were just large enough to be visible to the naked eye, which together indicate particle sizes on the order of 1 to 10 microns.

Quite probably the sonication breaks up the cubic phase into particles which are each actually a microcrystallite, because it is at the microcrystallite boundaries that the continuity of the polystyrene is probably most disturb, at these low concentrations of styrene in the cubic phase. Together all of these facts suggest that the size of the particles in the final dispersion could be controlled by controlling a) the nucleation kinetics and thus the microcrystallite size; b) the concentration of monomer and, in the particular, of cross-linking agent; and c)the extent of sonication. The density matching is then a relatively simple step, and in cases where particle flocculation is a problem, standard techniques in emulsion science can be used to stabilize the dispersion against flocculation, such as the use of surfactants or adsorbing polymers.

c) Spray techniques can be used, in which for example tiny amounts of lipid or surfactant would be sprayed into a liquid, most likely water or aqueous solution, this method applying at least in cases where the lipid or surfactant forms a cubic phase which is in equilibrium with excess water. For example, the polymerizable lipid glycerol monolinoleate (“monolinolein”, discussed above) forms a cubic phase which is in equilibrium with excess water over a wide temperature range, and therefore if a drop of monolinolein were introduced into and excess of water, it would spontaneously form a tiny clump of cubic phase, this being the equilibrium state. Such clumps could be then polymerized to form the desired dispersion of solid, microporous particles.

d) Another technique is to use a solvent, such as ethanol, in which the surfactant or lipid is soluble, and mix together a dilute surfactant solution with a dilute solution of water in the solvent then evaporate off the solvent. The solvent should of course be more volatile than water. Due to the high dilution of the surfactant, which should be chosen to form a cubic phase in equilibrium with water, nucleation processes result in very small clumps of cubic phase, and these can be polymerized either before or after the evaporation of the volatile solvent. Preliminary experiments at Lunds University have shown that dispersions of monoolein can be prepared in this way, although as yet polymerization has not been performed (e.g., by using monolinolein rather than monoolein) nor has it been demonstrated that the clumps are in fact cubic phase.

In such techniques there are at least two very general ways in which biocatalysts could be incorporated in the cubic phase particles. First, the catalyst could be covalently bonded, or adsorbed, etc., to the porewalls of the cubic phase particles in the dispersed state. And second, the cells or enzymes could themselves act as nucleation sites for the formation of cubic phase microcrystallites. Note that in the latter case the demands on the surfactant-catalyst interactions are very nonspecific, for the simple reason that in general the creation of nucleation sites by “impurities” does not require specific or permanent interactions at these nucleation sites. For example, water of very high purity can be undercooled many degrees below 0° C. whereas any of a wide range of impurities will significantly reduce this undercooling.

The use of such dispersions of polymerized cubic phase particles in first-order controlled-release drug delivery is an exciting possibility opened up by the present invention, as the following example shows. Consider the release of insulin in response to blood glucose levels. Particles could be prepared in which each particle had an outer coating consisting of a bicontinuous cubic phase laden with glucose oxidase. UV irradiation would proceed at least to the point where this outer coating was polymerized. In the presence of high glucose levels, the oxidation of glucose by the immobilized enzyme would cause a lowering of the pH due to the production of hydrogen peroxide. Then, methods are known by which pH changes can be used to effect the release of, for example, insulin.

This latter example illustrates a feature of the present invention which is independent of the primary feature of monodisperse pores. This feature is, namely, the fact that particles of a wide variety can be coated with bicontinuous cubic phase and polymerized to create an outer, microporous coating which can also contain biocatalysts. The high viscosity of cubic phases, together with the fact that many exist in equilibrium with excess water, make it possible to create the cubic phase coating under equilibrium conditions. If one were to try the same procedure with, for example, acrylamide, this would be impossible because the AM would be in solution and not on the surface of the particles.

### Containment of Biocatalysts Within Semipermeable Membrane Cells

Biocatalysts can be immobilized by placing a solution of the catalyst inside a cell which is used in the same way as a beaker but which is capable of continuous operation mode because of the use of a semipermeable membrane. The membrane should allow reactants and products to pass freely but should contain the biocatalyst inside the cell. Clearly the precision of the present microporous material could open up new possibilities in biocatalysis using this approach, both by increasing the effectiveness and reliability of existing processes, and by making feasible new combinations of catalyst and substrate which previously were not separable with existing membranes. As was discussed above, although the molecular weight of typical enzymes is usually considerably larger than that of their corresponding substrates, the effective “diameter” of each of these compounds goes roughly as the one-third power of the molecular weight, so that the ratio of the effective diameters of an enzyme to its substrate is usually much less than 10, and often only two or three. The requirements on the containing membrane are thus in many cases that the pores be substantially monodisperse.

This method is one of the only methods which is effective with high-molecular weight or water-insoluble substrates. Other methods, such as enzymes bound to water-insoluble polymers, have inherently low effectiveness because of the steric repulsion between the polymer and the substrate. In addition, in cases where the action of the enzyme is to breakdown a higher-MW substrate, the high monodispersity of the pores in the present materials can be used to control the molecular weight of the final product exiting from the reactor cell; with a smaller pore size, the substrate would be contained for a longer time in the cell and broken down into smaller fragments, until finally these were small enough to pass through the membrane.

In addition to size exclusion, porewall charge characteristics can be selected so as to retain the enzyme and allow passage of substrates and products. Above, many possible means for producing membranes with anionic, cationic, zwitterionic, polar, and nonpolar porewalls were discussed, and every year the number of successfully synthesized polymerizable surfactants increases, making more choices available for producing such membranes from polymerizable surfactants with desired electrostatic properties.

In this method of immobilization, there is not modification of the enzyme required, and in fact the enzyme is simply put into solution and placed inside the cell. After use, the enzyme solution can be removed and reused. Furthermore, several biocatalysts can be simultaneously immobilized, while minimizing the problems associated with other immobilization methods when faced with several enzymes having different chemical and physical requirements.

A related application of semipermeable membranes in the use of enzyme reactions is exemplified by the glucose prove produced by Yellow Springs Instrument Company. This probe consists of three layers placed in contact with a polarized platinum electrode; this electrode is sensitive to hydrogen peroxide. The glucose oxidase on glutaraldehyde resin particles constitutes the middle layer which lies between a polycarbonate and a cellulose acetate membrane. These membranes not only immobilize the enzyme, but they also minimize the amount of compounds reaching the probe electrode which would otherwise interfere with the measurement. The pore of the polycarbonate membrane allow the passage of glucose and oxygen, but not cells or macromolecules. the inner, cellulose acetate membrane allows hydrogen peroxide to reach the electrode but not glucose and acids such as uric or ascorbic acid. However, in view of the limitations of the cellulose acetate membrane, it is perhaps not too surprising that other substances, such as blood preservatives (Hall and Cook, 1982; Key and Taylor 1983) and certain drugs (Lindh et al. 1982) are able to reach the electrode where they produce spurious results. this example serves to demonstrate the potential importance of the present invention in biocatalysis applications due to its ability to exclude, on the basis of size, compounds which are not inert with respect to the catalysts or with associated probes.

It should also be noted that the importance of having available effective immobilization procedures for enzymes will likely become increasingly more important due to the fact that recombinant DNA technology is now making tailor-made enzymes possible. Other related areas in which the present invention could be of importance in enzyme technology are BioF.E.T.'s, and chemiluminometric assays, which make use of luciferinase enzymes to achieve very sensitive analyses.

For certain enzymes which are particularly sensitive to chemical conditions and might lose considerable activity if exposed to unfavorable conditions during the polymerization step, there are many ways in the present invention to avoid such exposure. discussed above is the process of forming the microporous polymer first, followed by covalent bonding or adsorption of the enzyme according to more or less standard to more or less standard methods. In fact, in the recent literature on polymerizable liposomes synthetic schemes have been reported for introducing functionality in the lipids and subsequently covalently bonding enzymes; for example, polymerizable phospholipids with latent aldehydes in the polar groups can be photopolymerized and subsequent bonded with o-chymotrypsin (S. Regen, M. Singh and N. K. P. Samuel 1984). Another method for bilayer-bound enzymes involves the use of lipids or surfactants which contain a polymerizable group as part of a spacer that extends out from the bilayer into the aqueous phase. Laschewsky, Ringsdorf, Schmidt and Schneider (1987) have synthesized several such polymerizable lipids, including one form that is a phospholipid. Even if radical-generating initiators were used to initiate the polymerization of such lipids, they could be chosen so as to reside in the aqueous phase and thus the exposure of the enzyme to any radicals would be minimal or essentially nonexistent. Two of the lipids synthesized by that group are, except for the polymerizable group, basically the same as the lipid glycerol monooleate (or monoolein), which as discussed at length above forms bicontinuous cubic phases; furthermore, as discussed herein some of these cubic phases are in equilibrium with excess water and thus very versatile and convenient in many respects.

Another method which involves remarkably mild conditions during polymerizin is through the use of lipids or surfactants forming sulfide linkages. Thiol bearing phosphotidylcholine lipids have synthesized (N. K. P. Samuel, M. Singh, K. Yamaguchi, and S. L. Regen 1985), and one variant is a cylic monomer with a disulfide bond. This cyclic monomer undergoes a ring-opening polymerization triggered by 5 mol % dithiothreitol (DTT). These authors claim that this is the mildest synthetic route available for the polymerization of phospholipid membranes. In addition, the fact that the number and type of chemical bonds in unchanged by the polymerization suggests that the change in volume upon polymerization should be very small, although the publications to date on these lipids do not discuss this. An small change in volume on polymerization is important in fabricating precision parts, and in maintaining polymer uniformity with a low density of defects.

These thiol-bearing phosphotidylcholine lipids can be polymerized and depolymerized by a thio-disulfide redox cycle: hence they have been referred to as ‘on-off’ surfactants. This opens up many exciting possibilities, including that of controlled-release applications. One such possibility now being discussed in the literature on liposomes is the controlled-release of antigens/haptens, because their lateral mobility and distribution are believed to play an important role in the immunological system (J. T. Lewis and H. M. McConnell 1978). It has been suggested that the lateral motion of haptens could be tuned through the use of vesicles composed of on-off lipids or surfactants. We suggest here that the same approach using bicontinuous cubic phases could be even more effective because of the inherently higher concentrations in cubic phases and the fact that cubic phases are thermodynamic equilibrium states, and can thus be produced under milder conditions and with more reliability and versatility in the process conditions. We have previously discussed conditions under which phospholipids are expected to form bicontinuous cubic phases.

These polymerizable/depolymerizable lipids are one example of polymerizable lipids which form polymers that are biodegradable. another class of such compounds now being investigated consists of lipids or phospholipids with amino acid groups which polycondensate to form polypeptides. As early as 1948, Katchalsky and coworkers performed a successful polycondensation reaction of octadecyl esters of glycine and analine in Langmuir-Blodgett multilayers. Such studies are now being actively resumed in an attempt to produce biodegradable polymerized vesicles, and as above we argue that similar chemistry, but carried out in the bicontinuous cubic phase instead of in vesicles, can be used to create biodegrable and/or controlled-release materials endowed with the inherent features of bicontinuous cubic phases.

Under the general heading of polymerizable surfactants, the polymerization of counterions is another interesting possibility for the fixation of biocatalyst-containing bicontinuous cubic phases, with a minimum of effect on bilayer-bound catalysts. The polymerization of counterions is similar in spirit to the use, in Nature, of polymeric frames that are attached to cell biomembranes and that lend the biomembrane an added degree of stability and flexibility. In fact, Mollerfeld et al. (J. Mollerfeld, W. Prass, H. Ringsdorf, H. Hamazaki, and J. Sunamoto 1987) showed that the mechanical stability of bilayers of glycerol monooleate (monoolein) can be dramatically increased by the introductions of hydrophobized polysaccharides. Polymerizable counterions, typically containing methacrylate groups, are now being investigated in connection with liposomes. Choline methacrylate counterions (H. Ringsdorf and R. Schlarb 1986) for double-tailed phosphates create analogues to phospholipids with polymerizable counterions. A further step is the anchoring of the resulting polyelectrolyte to the (unpolymerized) lipid by covalent bonding of the polyelectrolyte to some of the lipids. Work at the university of Lund has shown that the polymerization of counteriors leads to a tighter binding of the counterions to the coions, due to the reduced effect of the counterion translational entropy (C. Woodward, B. Jonsson 1988), and this effect could be expected to lead to greater mechanical stability.

### Program Forf

c This is for 21×21 meshes!!

c calculates form factor of a LFR of double diamond at

c reciprocal space lattice vectors. Face centered

c real space lattice used. Note that densities are

c 1-phi(in channels), -phi(in matrix), (and 0 outside LFR).

parameter (nn=20)

parameter (nnp=21)

implicit double precision(a-h,p-z)

dimension q(441),for(nnp,nnp,nnp),phi(4),n(4),gw(2)

2,j(3),amp(nnp nnp,nnp),h1(24,nnp,nnp,nnp),v(4),cv(4),w(4)

3,h2(24,nnp,nnp nnp),h3(24,nnp,nnp,nnp),mult(nnp,nnp,nnp)

dimension fv(3

pi=4.*atan(1.0)

dd=.05

c Jacobian is 1/16 with F43m unit cell.

c But since actual Pn3m region has Jacobian

c of ½ and has,volume 2, this form factor must

c be mutiplied by,4**2=16 to get energy p.u.v.

weigh=.0625/1600.

w(3)=0.0

w(4)=1.0

sr3=sqrt(3.)

gw(1)=(sr3-1.)/(2.*sr3)

gw(2)=(sr3+1.)/(2.*sr3)

open(unit=9,file=‘forle’)

open(unit=4,file=‘dless’)

fv(1)=.319918

fv(2)=.33698

fv(3)=.3560112

do 999 nd=1,3

vf=fv(nd)

c vf=1.0

vfm1.0-vf

read(4,4)(q(nm),nm=1,441)

4 format(3e26.14)

do 5 jj=0,nn

do 3 kk=0,nn

do 1 ll=0,nn

amp(jj+1,kk+1,ll+1)=0.0

c Note that actual Miller indices of for(jj+1,kk+1,ll+1)

c are 2*jj2*kk,2*ll, with fcc unit cell.

1 continue

3 continue

5 continue

do 50 jk1=0,nn

do 40 jk2=0,jk1

do 30 jk3=0,jk2

j(1)=jk1

j(2)=jk2

j(3)=jk3

do 31 n3=1,3

do 29 n2=1,2

mm1=2*n2−3+4*(2-n2)+n3

m1=mm1−3*((mm1−1)/3)

mm2=4*n2−6+4*(2-n2)+n3

m2=mm2−3*((mm2-1)/3

mm3=6*n2−9+4*(2-n2)+n3

m3=mm3−3*((mm3−1)/3)

c Loop over 4 inversions.

do 19 jb=1,4

if(jb.eq.4)go to 43

if(jb.eq.3)go to 33

if(jb.eq.2)go to 23

xm=1.0

ym=1.0

zm=1.0

go to 93

23 xm=−1.0

ym=−1.0

zm=1.0

go to 93

33 xm=−1.0

ym=1.0

zm=−1.0

go to 93

43 xm=1.0

ym=−1.0

zm=−1.0

c Note that wave vector is 2*pi*(2m1,2m2,2m3)

93 numh=6*(jb−1)+3*(n2−1)+n3

h1(numh,j(1)+1j(2)+1,j(3)+1)=4.*pi*j(m1)*xm

h2(numh,j(1)+1j(2)+1,j(3)+1)=4.*pi*j(m2)*ym

h3(numh,j(1)+1j(2)+1,j(3)+1)=4.*pi*j(m3)*zm

19 continue

29 continue

31 continue

30 continue

40 continue

50 continue

Loop over gauss points.

do 500 l1=1,2

ya=gw(l1)

do 400 l2=1,2

za=gw(l2)

phi(1)=(1.−ya)*(1.−za)

phi(2)=ya*(1.−za)

phi(3)=(1.−ya)*za

phi(4)=ya*za

do 300 ne=1,400,

mv=(ne−1)/20+1

mh=ne−20*(mv−1)

n(1)=ne+mv−1

n(2)=ne+mv

n(3)=ne+mv+20

n(4)=ne+mv+21

yl=(mh−1)*dd

zl=(mv−1)*dd

y=yl+ya*dd

z=zl+za*dd

fi=0.0

do 87 lx=1,4

nlx=n(lx)

fi=fi+q(nlx)*phi(lx)

87 continue

Note that this is the larger of w* and 1−w*(v,u)

w(2)=fi

nv=mh

nh=mv

nne=20*(nv−1)+nh

n(1)=nne+nv−1

n(2)=nne+nv+20

n(3)=nne+nv

n(4)=nne+nv+21

fi=0.0

do 88 lx=1,4

nlx=n(lx)

fi=fi+q(nlx)*phi(lx)

88 continue

w(1)=1.0-fi

do 73 k1=0,nn

do 72 k2=0,k1

do 71 k3=0,k2

ksum=k1+k2+k3

neven=ksum−2*ksum/2)

do 70 nf=1,24

hh1=h1(nf,k1+1,k2+1,k3+1)

hh2=h2(nf,k1+1,k2+1,k3+1)

hh3=h3(nf,k1+1,k2+1,k3+1)

a=.25*(hh1*(−y−z)+hh2*(y−z)+hh3*(2.−y−z))

b=.25*(hh1*(1+.y)+hh2*(1.−y)+hh3*(y−1.))

if(abs(a).lt.0.00001)go to 100

if(neven.ne.0)go to 81

do 89 mk=1,4

arg=a*w(mk)+b

v(mk)=(a*a*w(mk)*(1.0-w(mk))+2.0)*sin(arg)/(a*a*a)

2+(1.0−2.0*w(mk))*cos(arg)/a**2

89 continue

go to 80

81 do 84 mk=1,4

arg=a*w(mk)+b

v(mk)=−(a*a*w(mk)*(1.0−w(mk))+2.0)*cos(arg)/(a*a*a)

2+(1.0−2.0*w(mk))*sin(arg)a**2

84 continue

80 amp(k1+1,k2+1,k3+1)=amp(k1+1,k2+1,k3+1)+weigh*

2(vfm*(v(4)-v(2)+v(1)-v(3))-vf*(v(2)-v(1)))

go to 70

100 if(neven.ne.0)go to 64

amp(k1+1,k2+1,k3+1)=amp(k1+1,k2+1,k3+1)+weigh*

2((w(1)**2/2.-w(1)**3/3.)-(w(2)**2/2.-w(2)**3/3.)

3+vfm/6.)*cos(b)

go to 70

64 amp(k1+1,k2+1,k3+1)=amp(k1+1,k2+1,k3+1)+weigh*

2((w(1)**2/2.w(1)**3/3.)-(w(2)**2/2.-w(2)**3/3.)

3+vfm/6.)*sin(b)

70 continue

71 continue

72 continue

73 continue

300 continue

400 continue

500 continue

do 994 jj1=0,nn

do 993 jj2=0,jj1

do 992 jj3=0,jj2

am=amp(jj1+1,jj2+1jj3+1)

write(9,9)jj1,jj2,jj3,am

9 format(3i5,e20.8)

992 continue

993 continue

994 continue

999 continue

end

### Program Cruz

c This program computes, from the form factor

c of a Double-diamond surface, the total free

c energy for the double-diamond, lamellar,

c and cylindrical morphologies.

implicit double precision(a−h,o−z)

double precision MMBSJ1

dimension al(2),ef(2),d(2)

external MMBSJ1

open(unit=4,file=‘forless’)

pi=4.*atan(1.0)

th=1./3.

con=12.**th

print *, ‘enter (real) NO, and arm#’

read *, en0,arm

do 100 mp=1,4

nmax=1769

print *, ‘enter f and area’

read *, f,area

en=en0

ff=f*(1.−f)

al(1)=f

al(2)=1.−f

sf=1.−5*(arm−1.)*al(2)+5**ff *(arm−3.)

sum=0.0

do 90 nd=1,nmax

read(4,4)j,k,l,for

4 format(3i5,e20.8)

np=2

nq=2

nr=6

if(k.eq.0)np=1

if(l.eq.0)nq=1

if(j.eq.k)nr=3

if(k.eq.1)nr=3

if(j.eq.1)nr=1

mult=2*np*nq*nr

qs=4.*pi*pi*float(j*j+k*k+1*l)

x0=en0*qs/2.

do 5 mm=1,2

u=al(mm)*x0

d(mm)=al(mm)*al(mm)*(2./u**2)*

2(u+exp(−u)−1.)

ef(mm)=(1.−exp(−u))*al(mm)/u

5 continue

ep=exp(−al(1)*x0)

gq=(d(1)+d(2)+(arm−1.)*(ef(1)*ef(1)+

2ef(2)*ef(2)*ep*ep)+2.*ef(1)*ef(2)*(1.+

3(arm−1.)*ep))/

4(en0*en0*(d(1)*d(2)+(arm−1.)*(d(2)*ef(1)

5*ef(1)+d(1)*ef(2**ef(2)*ep*ep)-(ef(1)*ef(2))

6**2*(1.+2.*(arm−1.)*ep)))

fac=gq*en*en en*ff*ff/3.−en*en*qs*ff/12

2−en*sf/6.

sum=sum+mult*for*for*fac

90 continue

encub=(16.*sum)**th*con*area**(2.*th)/f

print *, ‘Energy for double-diamond Q* :’

print *, encub

c Now do lamellar phase

mmax=82

sum=0.0

do 95 nd=1,mmax

c Enter form factor here***

for=sin(pi*nd*f)/(pi*nd)

c Note that wave vector is 2*pi/D *(nd,0,0)

qs=4.*pi*pi*float(nd*nd)

x0=en0*qs/2.

do 6 mm=1,2

u=al(mm)*x0

d(mm)=al(mm)*al(mm)*(2./u**2)*

2(u+exp(−u)−1.)

ef(mm)=(1.−exp(−u))*al(mm)/u

6 continue

ep=exp(−al(1)*x0)

gq=(d(1)+d(2)+(arm−1.)*(ef(1)*ef(1)+

2ef(2)*ef(2)*ep*ep)+2.*ef(1)*ef(2)*(1.+

3(arm−1.)*ep))/

4(en0*en0*(d(1)*d(2)+(arm−1.)*(d(2)*ef(1)

5*ef(1)d(1)*ef(2)*ef(2)*ep*ep)-(ef(1)*ef(2))

6**2*(1.+2.*(arm−1.)*ep)))

fac=gq*e*en*en*ff*ff/3.−en*en*qs*ff/12.

2-en*sf/6.

sum=sum+for*for*fac

95 continue

sum=sum*24

enlam=sum**th/f

print *, ‘*’

print *, ‘Lamellar energy=Q* :’

print *, enlam

c Now compute total energy

c for cylindrical phase

sr3=sqrt(3.)

rad=sqrt(2.*f/(pi*sr3))

nmax=50

sum=0.0

do 89 ne=1,nmnax

do 80 nd=0,ne

ns=2

nb=2

if(ne.eq.ns)nb=1

if(nd.eq.0)ns=1

amult=float(2*ns*nb)

c Enter form factor here***

argg=rad*2.*pi*sqrt(float(nd*nd+nd*ne+ne*ne))

bes=MMBSJ1(argg,ier)

for=f*bes/argg

c Note that wave vector is 2*pi*(nd,ne,0)

qs=argg*argg/rad**2

x0=en0*qs/2.

do 15 mm=1,2

u=al(mm)*x0

d(mm)=al(mm)*al(mm)*(2./u**2)*

2(u+exp(−u)−1.)

ef(mm)=(1.−exp(−u))*al(mm)/u

15 continue

ep=exp(−al(1)*x0)

gq=(d(1)+d(2)+(arm−1.)*(ef(1)*ef(1)+

2ef(2)*ef(2)*ep*ep)+2.*ef(1)*ef(2)*(1.+

3(arm−1.)*ep))/

4(en0*en0*(d(1)*d(2)+(arm−1.)*(d(2)*ef(1)

5*ef(1)+d(1)*ef(2)*ef(2)*ep*ep)−(ef(1)*ef(2))

6**2*(1.+2.*(arm−1.)*ep)))

fac=gq*en*en*en*ff*ff*/3.en*en*qs*ff/12.

2−en*sf/6.

sum=sum+for*for*fac*amult

80 continue

89 continue

sum=sum*24.

encyl=(3.*sum/(f*rad*rad))**th

print *, ‘*’

print *, ‘Cylindrical energy=Q* :’

print *, encyl

print *, ‘*’

100 continue

end

### Further Summary Description of Invention

### Polymerization of Lyotropic Liquid Crystals

The polymerization of one or more components of a lyotropic liquid crystal in such a way as to preserve and fixate the microstructure has recently been successfully performed, opening up new avenues for the study and technological application of these periodic microstructures. Of particular importance are so-called bicontinuous cubic phases, having triply-periodic microstructures in which aqueous and hydrocarbon components are simultaneously continuous. It is shown that the polymerization off one of these components, followed by removal of the liquid components, leads to the first microporous polymeric material exhibiting a continuous, triply-periodic porespace with monodisperse, nanometer-sized pores.

The following section focuses on the fixation of lyotropic liquid crystalline phases by the polymerization of one (or more) component(s) following equilibration of the phase. The primary emphasis will be on the polymerization of bicontinuous cubic phases, a particular class of liquid crystals which exhibit simultaneous continuity of hydrophilic—usually aqueous—and hydrophobic—typically hydrocarbon—components, a property known as ‘bicontinuity’(Scriven, L. E., Nature 263:123 (1976)), together with cubic crystallographic symmetry (Luzzati et al., Biological Membranes; pp. 71-123 Academic: New York (1968)). The potential technological impact of such a process lies in the fact that after polymerization of one component to form a continuous polymeric matrix, removal of the other component creates a microporous material with a highly-branched, monodisperse, triply-periodic porespace.

While there have been efforts to polymerize other surfactant mesophases and metastable phases, bicontinuous cubic phases have only very recently been the subject of polymerization work. Through the use of polymerizable surfactants, and aqueous vesicles (Regen et al., J. Am. Chem. Soc. 102):6638 (1980), Fendler, J. H., Acc. Chem. Res. 17:3 (1984), Hub et al, Angew. Chem. 92(11):962 (1980), Johnston et al, Biochim. Biophys. Acta. 602:57 (1980) and Lopez et al., J. Am. Chem. Soc. 104:305 (1982)), surfactant foams (Friberg et al., J. Coll. Int. Sci. 118:543 (1987), inverted micellar solutions (Candau et al., J. Coll. Int. Sci. 101(1):167 (1984)), hexagonal phase liquid crystals (Thunathil et al., J. Polymer Sci. 18:2629 (1980)) and bicontinuous microemulsions (Candau et al., J. Col. Int. Sci. 114(2):398 (1986)). In the latter two cases rearrangement off the microstructure occurred during polymerization, which in the case of bicontinuous microemulsions seem inevitable because microemulsions are of low viscosity and continually rearranging on the timescale of microseconds due to thermal disruption (Lindman et al., Surfactants in Solution, Mittal et al, Eds.; Plenum New York, Vol. 3, p. 1651 (1984). In contrast, bicontinuous cubic phases are extremely viscous in general, and although the components display self-diffusion rates comparable to those in bulk, their diffusion nevertheless conforms to the periodic microstructure which is rearranging only very slowly. In fact, recently cubic phases have been prepared which display single-crystal X-ray patterns (Rancon et al., J. Phys. 48:1067 (1987)). In the author's laboratory, experiments are not performed in which bicontinuous cubic phases are routinely polymerized without loss of cubic crystallographic order. The fact that, in spite of the high viscosity) and high degree of periodic order, bicontinuous cubic phases have only recently been the focus of polymerization experiments can be traced to several causes, most notably: a) cubic phases cannot be detected by optical textures and usually exist over quite narrow concentration ranges; b) the visualization and understanding off the bicontinuous cubic phase microstructures pose difficult mathematical problems; and c) the focus of research on cubic phases has been on binary systems, in particular on biological lipid/water systems, whereas the best cubic phases from the standpoint of straightforward polymerization experiments are ternary surfactant/water/hydrophobic monomer systems.

As is clearly discussed in a recent review of polymerized liposomes (Regen et al., Liposomes: From Biophysics to Therapeudics; Marcel Dekker:

New York, pp. 73-108 (1987)), a distinction must be drawn between polymerized and polymeric surfactant microstructures. In Polymeric microstructures, the polymerization is carried out before the preparation of the phase, whereas the term polymerized means that the microstructure is formed first, and then the polymerization reaction performed with the aim of fixating the microstructure as formed by the monomeric components. Although this chapter deals mainly with polymerized microstructures, polymeric cubic phases are discussed in a separate section at the end.

The next section and the final section on polymeric cubic phases are intended for those readers who seek a more in-depth understanding of the microstructures involved, including the geometrical aspects as well as the physics behind the self-assembly into these structures. These sections may be omitted by the more casual reader.

### The Bicontinuous Cubic Phases: Mathematical Principles

An understanding of the basic mathematical principles that apply to the physics and the geometry of the bicontinuous cubic phases is necessary for full appreciation of what follows. Since 1976 (Scriven, L. E., Nature 263:123 (1976)), it has been known that a complete understanding of bicontinuous cubic phases requires an understanding of Differential Geometry and in particular, of a class of mathematical surfaces known as periodic minimal surfaces (often referred to as IPMS, for infinite periodic minimal surfaces; clearly ‘infinite’ is redundant). A minimal surface is defined to be a surface of everywhere zero mean curvature; the mean curvature at a point on a surface is one-half the sum of the (signed) principle curvatures, so that every point on a minimal surface is balanced saddle point: K₁=−K₂. The utility of periodic minimal surfaces of cubic symmetry, and of their constant mean curvature relatives, in the understanding of bicontinuous cubic phases is now well-established, and we begin with a short introduction to these surfaces. There has been considerable confusion in the literature over these complicated surfaces and even of their fundamental basis in the field of surfactant microstructures, but in the last few years this has become considerably clarified.

The first source of confusion was the fact that minimal surfaces represent local minima in surface area under Plateau (or ‘fixed boundary’) boundary conditions. The importance of this property with respect to cubic phases must be considered to be limited, however, because the surface area of the interfacial dividing surface drawn between the hydrophilic and the hydrophobic regions of the microstructure—is given simply by the product of the number of surfactant molecules, times the average area per surfactant which is strongly fixed by the stearic and electrostatic interactions between surfactant molecules. Therefore this interfacial area does not in general seek a minimum but rather an optimum value, which does not tend to zero because of the electrostatic repulsion between surfactant head groups. Furthermore, the fixed boundary conditions that lead to minimal surfaces are not as appropriate as boundary conditions which result upon enforcement of the volume fractions of the hydrophilic and hydrophobic moieties in the unit cell. Minimization of area under such constraints leads to surfaces of constant mean curvature—or ′H-surfaces—which can possess significantly lower interfacial areas than the corresponding minimal surfaces of the same symmetry and topological type (Anderson, D. M., Ph.D. Thesis, University of Minnesota, Minneapolis (1986)).

The traditional microstructures—spheres, cylinders, and lamellae—all have constant mean curvature dividing surfaces, and, as discussed below, the same appears to be true for bicontinuous cubic phases. However, at the same volume fraction, the different competing microstructures given rise to different values of the mean curvature, and a belief that is now firmly embedded in the study of surfactant microstructures is that the structure which is most favorable under given conditions is that which satisfies most closely the ‘preferred’ or ‘spontaneous’ mean curvature (Helfrich, W., Z. Naturforsch 28c:693 (1973)). The spontaneous mean curvature is determined by the balance of forces—stearic, electrostatic, etc.—between the surfactant head groups, and between the surfactant tails, and thus is sensitive to e.g., salinity, oil penetration, etc. In the liquid crystals of interest here, the surfactant-rich film is tending toward a homogenous state in which each surfactant molecule sees the same local environment, regardless of where on the monolayer it is located and, if this monolayer is one-half of a bilayer, regardless of which side of the bilayer it is on. (In certain biological systems there is a significant asymmetry with respect to the two sides of the bilayer, which is of great importance; however, we are dealing for the moment with the symmetric bilayer). Thus each monolayer is driven toward the most homogeneous state which implies a constant mean curvature.

A second source of confusion that still persists to some extent in the literature is the matter of where the interfacial surface is to be drawn. For those cubic phase structures discussed below in which a bilayer is draped over a minimal surface, this minimal surface describes the midplane (or better, ‘midsurface’) of the bilayer and not the interface between polar and apolar regions; that is, it describes the location of the terminal methyl groups on the surfactant tails, not the dividing point between the hydrophilic head group and the hydrophobic (usually hydrocarbon) tail. The actual polar/apolar dividing surface is displace d from the minimal surface by the length of the hydrophobic tail, on booth side of the minimal surface. While it can be argued as to exactly where in the bilayer profile these two polar/apolar dividing surfaces should be drawn, it is clear than any sensible convention should place them near the first methyl group in the tail and not at the terminal methyl at the tail end. Thus bilayer cubic phases should not be referred to as having a zero mean curvature interface.

Recently, a application of geometry and differential geometry to this problem has treated these matters quantitatively. For the case of a cubic phase whose local structure is that of a bilayer, then it has been shown (Charvolin et al., J. Physique 48:1559 (1987)) that the requirement of symmetry with respect to the two sides of the bilayer, and therefore of the two aqueous networks lying on the two sides of the surface, leads directly to minimal surfaces as midplace surfaces, and through a construction involving projections of surfaces in four-dimensional space leads to the minimal surfaces which describe in known bilayer cubic phases. Concerning the shape of the polar/apolar interface in such structures, the mean curvature cannot be identically zero, and here two cases must be distinguished. In normal cubic phases, which usually lie between lamellar and normal hexagonal phases, the mean curvature of the interface is on the average toward the hydrophobic regions, and these regions are well-described by interconnected cylinders. The axes of these cylinders are the edges of the two graphs (referred to as ‘skeletal graphs’in reference (Schoen, A. H., Infinite Periodic Minimal Surfaces Without Self-Intersections: NASA Technical Note D-5541 (1970); Natl. Tech. Information Service Document N70-29782, Springfield, Va. 22161); see also FIG. 10 below) that thread the two hydropic subspaces. These cylinders satisfy both constant mean curvature at the interface and a constant stretch distance for the surfactant tails (except at the junctions of the cylinders). However, in the inverted cubic phases, usually lying between lamellar and inverted hexagonal phases, the constant mean curvature and constant distance surfaces do not coincide. This situation has been referred to as ‘frustration’ (Charvolin et al. J. Physique 48:559 (1987)). Recently, the constant mean curvature configurations have been computed (Anderson, D. M., Ph.D. Thesis, University of Minnesota, Minneapolis (1986)), and shown to have rather mild variations in the stretch distance (Anderson et al., Proc. Natl. Acad. Sci., in press), which is measured from the minimal surface to the corresponding point on the constant mean curvature surface.

### The Bicontinuous Cubic Phase Microstructures

We have seen that the balance of forces on the hydrophilic and hydrophobic sides of the surfactant-rich film in a bicontinuous cubic phase determines a ‘preferred’ or ‘spontaneous’ mean curvature of the film, measured at the imaginary hydrophilic/hydrophobic dividing surface, so that the optimal shape of this dividing surface is tending toward a homogeneous state of constant mean curvature. In the case where the basic building block of the cubic phase is a surfactant bilayer—the usual case is binary lipid-water systems—there is in addition another imaginary surface that describes the midplane (or midsurface) of the bilayer, and this surface must be a minimal surface by symmetry considerations. In this section we discuss each of the known bicontinuous cubic phase microstructures, with the aid of computer graphics that will demonstrate these principles in a visual way.

### A Representative Bilayer Structure

An example of a constant mean curvature surface is shown in FIG. 10A, together with two skeletal graphs. The surface shown has diamond cubic symmetry, space group #216. One must imagine an identical copy of the surface shown as being displaced so as to surround the other skeletal graph, leading to a double-diamond symmetry, space group Pn3m, #224. One form of inverted cubic phase has this Pn3m symmetry with water located the two networks lying ‘inside’ the two surfaces, and the surfactant hydrocarbon tails in the ‘matrix’ between these two networks, with the two surfaces themselves describing the location of the surfactant head groups, or more precisely the polar/apolar interface. A triply-periodic minimal surface, known as Schwarz's Diamond (or D) minimal surface (Schwarz, H. A., Gesammelte mathematische Abhandlungen; Springer: Berlin, Vol.1 (1890)), shown in FIG. 10B, can then be imagined as bisecting the hydrocarbon region. Calculations show that the standard deviation of the stretch distance, from each point on the polar/apolar dividing surface to the minimal surface, is only about 7% of the average distance (Anderson et al., Proc. Natl. Acad. Sci., in press). In the actual cubic phase, the constancy of the mean curvature of the interface might be compromised somewhat in order to achieve even more uniformity in the stretch distance. This would not, however, affect the average value of the mean curvature (Anderson et al., J. Phys. Chem. (submitted)), which is significantly toward the water.

If, on the other hand, the double-diamond symmetry were found in a normal cubic phase, with mean curvature on the average toward the hydrocarbon regions, then one would expect to find that the polar/apolar interfacial surface shown in FIG. 10A would look instead like interconnected cylindrical rods, because the necks and bulges in FIG. 10A would not correspond to water channels but rather to channels occupied by surfactant tails with a preferred stretch distance. Thus far, such a normal cubic phase has not been observed with this symmetry, but has with another symmetry discussed below (#230), and the principles are exactly the same.

It has recently been established (see below) that upon the addition of a protein, for example, to such a structure, a variant of the structure can form in which one of the two water networks is replaced (at least in part) by inverted micelles containing hydrated protein. This changes the space group of the structure, for example #224 changes to #217.

### A Monolayer Structure

The author has proposed another structure of quite a different nature or a cubic phase occurring in ternary systems involving quaternary ammonium surfactants (Anderson, D. M., Ph.D. Thesis, University of Minnesota, Minneapolis (1986)), and this cubic phase is the focus of much of the polymerization work that has been performed. The surfactant didodecyldimethylammonium bromide (DDAB), together with water and a variety of oils, forms a cubic phase whose location is shown in FIG. 11 for the case of hexene. Thus the cubic phase exists over a wide range of DDAB/water ratios, but requires a minimum amount of hexene. The same is true for a large number of ‘oils’ that have been investigated, including alkanes from hexene to tetradecane, alkenes, cyclohexane (Fontell et al., in preparation), and monomers such as methylmethacrylate (MMA) and styrene (Anderson, D. M., U.S. patent appln. Ser. No. 32,178, EPO Patent Appln. 88/304,625.2 and Japanese Patent Appln. No. 63-122,193 (1987)). The fact that the cubic phase region extends very close in composition to the L₂ phase region, but not as far as the binary surfactant/water edge, suggests that in this structure the surfactant is locally in the form of a monolayer rather than a bilayer.

The model proposed by the author for this cubic phase is shown, for the case of aqueous volume fraction equal to 47%, in FIG. 12. One must imagine the oil and the surfactant tails being located on the ‘inside’ of the dividing surface, water and counterions on the ‘outside’, and the quaternary ammonium head groups located at or near the depicted surface. The space group is Im3m, #229, which is the same as one of the bilayer cubic structures described below, but these two structures are very difference even though the indexing of the X-ray patters is the same. This structure will be referred to as the ‘I-WP’ structure (Anderson D. M., Ph.D. Thesis, University of Minnesota, Minneapolis (1986); Schoen, A. H., Infinite Periodic Minimal Surfaces Without Self-Intersections: NASA Technical Note D-5541 (1970); Natl. Tech. Information Service Document N70-29782, Springfield, Va. 22161), because the two skeletal graphs are the BCC or ‘I’ graph (threading the hydrophobic labyrinth) and the ‘wrapped package’ or ‘WP’ graph (threading the hydrophilic labyrinth). In FIG. 13 are shown three structures in the continuous, one-parameter family (not counting variations in lattice parameter) of I-WP structures, which correspond to aqueous volume fractions of: a) 30%; b) 47%; and c) 65%. This family of constant mean curvature surfaces (Anderson, D. M., Ph.D. Thesis, University of Minnesota, Minneapolis (1986)) is proposed to represent the progression in structure as the water/surfactant+oil ratio is increased; there is also an increase in lattice parameter with increasing water content, from just under 10 Å at low water to about 300 Å at the highest water contents. This family of structure models is supported by the following evidence:

1) The indexing and relative peak intensities in SAXS patterns from the cubic phase are fit well by the I-WP model, but not by alternative models (Anderson, D. M., Ph.D. Thesis, University of Minnesota, Minneapolis (1986));

2) TEM micrographs of a polymerized cubic phase match theoretical simulations using the model (Anderson, D. M., U.S. patent appln. Ser. No. 32,178, EPO Patent Appln. No. 88/304,625.2, and Japanese Patent Appln. No. 63-122183 (1987)), but not alternative models (see below);

3) Pulse gradient NMR self-diffusion date (Fontell in preparation) correlate well with theoretical calculations, in which the diffusion equation was solved in the model geometries by a finite element method (Anderson Wennerstrom in preparation);

4) Values of the area per surfactant head group, calculated from the SAXS lattice parameters assuming the I-WP models, increase from 47 Å to 54 Å² as the water fraction increased from 30% to 65% thus increasing the head group hydration; this compares well with a value of 54 Å² for the inverted hexagonal phase very near in composition;

5) The calculated mean curvature of the monolayer goes from toward water at low water content, through zero, to toward oil continuously as the water content increases from less than to greater than 50%; this is well-known in ternary microemulsion systems, and is very hard to reconcile with a bilayer model; furthermore, the mean curvature values in the inverted hexagonal phase at higher oil/surfactant concentration are more toward the water, which fits well with the idea of increased curvature toward water with increasing penetration of oil into the tail region of the monolayer (Ninham et al., J. Phys. Chem. 88:5855 (1984));

6) The wide range of hydrophobe/hydrophile ratios in the cubic phase region is also difficult to reconcile with a bilayer model, and in fact has never been observed to this extent in any bilayer cubic phase, but it is readily explained by the progression depicted in FIG. 13;

7) The proposed structure at low water content, shown in FIG. 13A, ties in very well with the microstructure that is now generally accepted for the low-water-content microemulsions in the near by L₂ phase region, namely a bicontinuous, monolayer structure with water lying inside a network of interconnected tubules.

### The Known Bicontinuous Cubic Phase Structures

Recording the structures that have been proposed for bicontinuous cubic phases:

#224, with the Schwarz Diamond minimal surface describing the midplane of a bilayer; also known as the ‘double-diamond’ structure, well-established in the glycerol monooleate (GMO or monoolein)/water system (Longley et al., Nature 303:612 (1983)), described in detail above; the double-diamond structure is also found in block copolymers (Thomas et al., Macromolecules 19(8):2197 (1986) and Anderson et al., Macromolecules, in press) (see the final section).

#227, obtained from #224 by replacing one of the water labyrinths with inverted micelles; observed when oleic acid is added to monoolein/water at acidic pH (Mariani et al., J. Mol. Biol. in press).

#229, the space group of two distinct structures:

a) the bilayer structure with the Schwarz Primitive minimal surface describing the midplane of a bilayer; this minimal surface has six ‘arms’ protruding through the faces of each cube; this structure has been more difficult to establish unambiguously, but appears to occur in monoolein/water systems and with a added cytochrome (Mariani et al., J. Mol. Biol. in press), and in sodium dodecyl sulphate/water (Kekicheff et al., J. Phys. 48:1571 (1987)).

b) the I=WP monolayer cubic phase described in detail above.

#230, with Schoen's ‘gyroid’ minimal surface (Schoen, A. H., Infinite Periodic Minimal Surfaces Without Self-Intersections: (NASA Technical Note D-5541) (1970); Natl. Tech. Information Service Document N70-29782 Springfield, Va. 22161) describing the midplane of a bilayer (Hyde et al., Z. Krist 168:213 (1984) the two water networks in this structure are enantiomorphic, and characterized by screw symmetries rather than reflectional or rotational; this appears to be the most common cubic structure, at least in lipids; the normal form of this structure also exists, in which the two enantiomorphic networks are filled with surfactant, and the minimal surface is the midplane of an aqueous network; this normal form occurs in some simple soaps (Luzzati et al., Nature 220:485 (1968)).

#212, obtained from #230 by replacing one of the water labyrinths with inverted micelles; this is the only known cubic phase with a non-centrosymmetric space group; found in the monoolein/water/cytochrome-c system (Mariani et. al, J. Mol. Biol., in press), and also by the author at the same composition but with monolinolein replacing monoolein (see below).

It is interesting to note that, in contrast to the number of bicontinuous cubic phase structures which apparently exist, only one cubic phase structure is now recognized that is not bicontinuous. Furthermore, this structure does not consist of FCC close-packed micelles, but rather a complicated packing of nonspherical micelles (Fontell et al., Mol. Cryst. Liq. Cryst. 1(1,2):9(1985)).

### Preparation and Characterization of Polymerized Cubic Phases

The first bicontinuous cubic phases too be polymerized (Anderson, D. M., U.S. patent appln. Ser. No. 32,178; EPO Patent Appln. No. 66/304,625.2 and Japanese Patent Appln. No. 63-122,193 (1987)) were the ternary DDAB/water/hydrophobic monomer phases described above, which were interpreted as having the ‘I-WP’ structure. This surfactant was chosen primarily because it was previously known to form bicontinuous phases—cubic phases and microemulsions—with many oils or oil-like compounds, including hexane through tetradecane (Mariani et al., J. Mol. Biol., in press), alkenes (Ninham et al., J. Phys. Chem. 88:5855 (1984)), cyclohexane (Chen et al., J. Phys. Chem. 90:842 (1986)) brominated alkanes (present author, unpublished), and mixtures of alkanes (Chen et al., J. Phys. Chem. 90:842 (1986)). The location of the cubic phase region in these various systems is rather independent of the choice of hydrophobe, which suggests that the hydrophobe is largely confined to (continuous) hydrophobic channels, having little direct effect on the interactions in the head group region. This makes it an ideal system for investigating polymerization by substituting a hydrophobic monomer.

The composition chosen for the initial experiments was 55.0% DDAB, 35.0% water, and 10.0% methylmethacrylate (MMA), which had been purified by vacuum distillation and to which had been added 0.004 mg/ml of the initiator azobisisobutyronitrile (AIBN). Upon stirring the solution became highly viscous and showed optical isotropy through crossed polarizers, two signs characteristic of the cubic phase (an early name for the cubic phase was in fact the ‘viscous isotropic phase’). With other oils such as decane, this composition yields a bicontinuous cubic phase, as indicated by SAXS (Anderson, D. M., Ph.D. Thesis, University of Minnesota, Minneapolis (1986) and Fontell, et al. Acta Chem. Scand. A40:247 (1986)) and NMR self-diffusion (Fontell, et al., Acta Chem. Scand. A40:247 (1986)). After equilibrating for one week at 23° C., two samples were prepared for polymerization. The first sample was prepared for SAXS; the phase was smeared onto the end of the plunger of a large syringe, and pushed through an 18 gauge needle into 1.5 mm i.d. X-ray capillary. The second sample was loaded into a quartz, water-jacketed reaction cell, and nitrogen gas was continually pumped over the sample.

The capillary and the quartz cell were placed in a photochemical reactor having four 340 nm UV lamps, for 36 hours of exposure. At the end of this time the samples were opaque while in appearance. The second sample could be rendered clear by the use of a refractive-index matching fluid. To do this, first a large amount of ethanol was used to remove the DDAB, the water, and monomeric MMA. Then the sample was dried in a vacuum oven, to yield a solid but highly porous material. Butyl benzene, which has a refractive index (n=1.4898 at 20° C.) very close to that of PMMA (1.4893 at 23° C.), was imbibed into the porous material, thereby rendering it clear. Upon drying off the butyl benzene, the material once again turned opaque. This is apparently a result of microcrystallites whose sizes are on the order of the wavelength of light; at this low volume fraction of monomer (10.0%), it is easy to imagine that the homogeneity of the polymerized PMMA could be disturbed at the microcrystallite boundaries. Below a system is discussed that yields clear materials.

The polymerized sample in the capillary was examined with the modified Kratky Small-Angle X-Ray camera at the University of Minnesota. Due to beam-time limitations (five hours, at 1000 Watts of Cu K∝ radiation), the statistics in the data re not particularly good, but (FIG. 14) clearly long-range order is indicated by the presence of Bragg peaks, which are indexed to a BCC lattice in FIG. 14, the lattice parameter being 118 Å. The maintenance of cubic crystallographic order through polymerization has also been confirmed recently in K. Fontell's laboratory. The capillary used in the Kratky camera was broken open and the components placed in ethanol, and the insoluble PMMA removed and weighed to confirm polymerization.

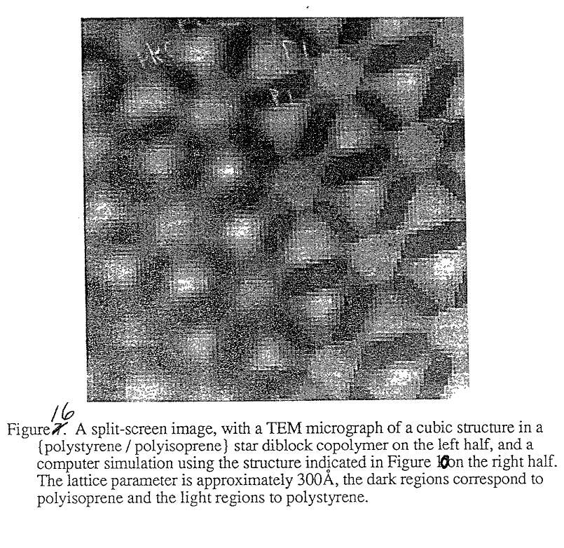

The standard method for visualization of microporous polymeric materials is to dry the sample with supercritical drying, which dries the pores without exposing them to the disruptive surface tension forces associated with normal evaporation. However, due in part to equipment problems, and in part to the small scale of the pores, this has not yet been performed on a polymerized cubic phase. Transmission electron microscopy has, however, been performed on an air-dried sample. The second sample above was ultramicrotomed at room temperature, and examined in a Jeol 100 CX electron microscope operating at 100 KV in TEM mode. Not only the drying process but also, of course,the microtoming procedure have strong disruptive effects on this highly-porous material. Nevertheless, the resulting micrograph (FIG. 15A; magnification 1,000,000×) indicates regions of periodic order, and in fact the entire field of view in the micrograph gives indications of being a (disrupted) single microcrystallite. An optical transform of the negative also substantiated the cubic symmetry. FIG. 15B is a simulation of the micrograph using the ‘I-WP’ model structure; a (111) projection of the model structure was calculated by computer, but sending rays through the model and calculating the portion of each ray that lies in void, and in polymer.

### Incorporation of Proteins into the Polymerized Structures

Experiments are now being performed in which proteins, and in particular enzymes, are incorporated into bicontinuous cubic phases and the resulting reaction medium permanented by polymerization. It is well established that the activity and stability of enzymes are generally optimal when the environment of the enzyme is closest to the natural in vivo environment, and the lipid bilayer that makes up bicontinuous cubic phases is the normal environment of functioning integral proteins. Polymerization of this continuous bilayer, one example of which is described below, creates by virtue of the bicontinuity a solid, microporous material that allows continuous flow of reactants and products. Furthermore the environment of the protein is precisely controlled sterically and electrostatically, as well as chemically. Control of the geometry of the porespace could be utilized to bias the registry between the enzyme and substrate toward the optimal orientation and proximity, in addition to providing further control of the chemistry by selection on the basis of molecular size. The electrostatic nature of the porewalls is very homogeneous due to the strong tendency for lipid polar groups to maintain an optimal separation, and it is known that the specificity of many enzymes is sensitive to changes in net charge. In addition the biocompatibility of the presently described materials render them of potential importance in controlled-release and extracorporeal circuit applications.

### Immobilization of Glucose Oxidase

The enzyme glucose oxidase was incorporated into the aqueous phase of a cubic phase similar to that polymerized in the previous section, and this aqueous phase polymerized by the addition of monomeric acrylamide. Except for a slight yellowish color from the strongly-colored glucose oxidase, the result was an optically clear polymerized material. The concentration of enzyme in the aqueous phase was 10.3 mg/ml, the acrylamide concentration was 15.4 wt %, and hydrogen peroxide as initiator was present at 0.3 w/w % of the monomer. This aqueous solution was mixed in a nitrogen atmosphere with 24.3 wt % DDAB and 10.93 wt % decane, and the solution centrifuged for one hour to remove any remaining oxygen. This water content, 64.8%, was chosen based on SAXS studies of the cubic phase as a function of water content in similar systems (Anderson, D. M., Ph.D. Thesis, University of Minnesota, Minneapolis (1986); also K. Fontell, unpublished) Above about 63 vol % water, the lattice parameter is larger than 175 Å with either decane or decanol, and according to the model shown in FIG. 13C the aqueous regions should be large enough to contain the enzyme.

Two samples were prepared for polymerization. One sample was simply placed in a quartz tube and polymerized for X-ray analysis. The other was smeared onto a nylon backing which had been shaped to fit on the end of a pH probe. Both samples were bathed in nitrogen during UV irradiation. The first sample was about 1.5 mm thick and after polymerization was a clear solid which could be handled easily; this was loaded into a flat SAXS cell with mica windows. Indexing of the resulting peaks to a BCC lattice indicated a lattice parameter of 320 Å. The second polymerized sample was soaked for one day in ethanol to remove the DDAB and decane, and then secured over the tip of a pH probe, and the enzyme was shown by the method of Nilsson et al. (Nilsson et al., Biochim. Biophys. Acta. 320:529 (1973)) to have retained its activity in the polymerized cubic phase. This example was intended only or demonstration of a general application, namely in biosensors, and is not particularly impressive in itself because a simple polyacrylamide gel has enough porosity to pass glucose. Nevertheless, in many cases the substrates to be detected are of higher molecular weight than glucose and the porespace created by the cubic phase microstructure can be tailored to the size of the substrate. In the next example the porosity is due solely to the cubic phase microstructure.

### Enzyme Immobilized in a Lipid-water Cubic Phase

At the time of this report the author is completing an experiment which demonstrates that proteins can be incorporated, in fairly high concentrations, into bicontinuous cubic phases made with polymerizable lipids that are biocompatible. Glycerol monooleate, or ∝-monoolein, is an uncharged, biocompatible lipid (the β-form is found in mushrooms), with one fatty acid chain containing a single double bond. A variant of monoolein with a conjugated diene in the chain is monolinolein, and the monolinolein-water phase diagram is known to be nearly identical with that of monoolein-water (Lutton, E. S., J. Am. Oil Chem. Soc. 42:1068 (1966)). As discussed above, the #212 cubic phase structure has been found in the monoolein/water/cytochrome-c system, and the present author has found the same structure at 6.7 wt % cytochrome, 14.8% water, and 78.5% monolinolein, where the monolinolein contains 0.4% AIBN. After equilibration, this cubic phase was placed in the UV photochemical reactor in a water-jacketed cell and bathed in nitrogen in the usual manner. After 48 hours the sample had polymerized and could be held by a tweezers, and was a deep red color, as in the unpolymerized phase, due to the strongly-colored protein. Presently work is under way to further characterize this material.

### Potential Technological Applications

The polymerization of bicontinuous cubic phases provides a new class of microporous materials with properties that have never before been attainable in polymeric membranes. The most important of these properties are now discussed in turn, and for each an application is briefly discussed to illustrate the potential importance of the property in a technological, research, or clinical application.

1) All cells (pore bodies) and all pore throats are identical in both size and shape, and the sizes and shapes are controlled by the selection of the composition and molecular weights of the components, over a size range which includes that from to 10-250 Å pore diameter and potentially into the micron range. Cell shape cover a range including that from substantially cylindrical to spherical, and cell diameter-to-pore diameter ratios which cover a range including that from 1 to 5, and connectivities which cover a range including that from 3 to 8 pore throats emanating from each cell.

Application: Clearly one important application of microporous materials in which the effectiveness is critically dependent on the monodispersity of the pores is the sieving of proteins. In order that an ultrafiltration membrane have high selectivity for proteins on the basis of size, the pore dimensions must first of all be on the order of 25-200 Å, which is an order of magnitude smaller than the smallest pore dimensions of typical microporous materials. In addition to this, one important goal in the field of microporous materials is the attainment of the narrowest possible pore size distribution, enabling isolation of proteins of a very specific size, for example. Unless, as in the present material, the pores are all exactly identical in size and shape, then in any attempt to separate molecules or particles on the basis of size, the effectiveness will be reduced when particles desired in the filtrate are trapped by pores smaller than the design dimension or oddly-shaped, and when particles not desired in the filtrate pass through more voluminous pores. Applications in which separation of proteins by molecular weight are of proven or potential importance are immunoadsorption process, hemodialysis, purification of proteins, and microencapsulation of functionally-specific cells.

2) The porespace comprises an isotropic, triply-periodic cellular structure. No prior microporous polymeric material, and no prior microporous material of any composition with pore dimensions larger than 2 nanometers, has exhibited this level of perfection and uniformity.

Application: Recently the author has become involved with studies of superfluid transitions which require microporous materials exhibiting long-range, triply-periodic order. In the Laboratory off Atomic and Solid State Physics at Cornell University, a group lead by Dr. John D. Reppy has been investigating the critical behavior of liquid ⁴He in microporous media (Chan et al., Phys. Rev. Lett., submitted). Certain theoretical treatments have predicted that the critical exponents characterizing the fluid—superfluid transition are different for disordered than for periodic porous media. The experiments described in the paper now being submitted for publication were performed using disordered media: Vycor, aerogel, and xerogel. The group is now proceeding on to a parallel set of experiments using the ordered microporous medium described herein.

3) In certain forms of the material, the microproous polymer creates exactly two distinct, interwoven but disconnected porespace labyrinths, separated by a continuous polymeric dividing wall, thus opening up the possibility of performing enzymatic, catalytic or photosynthetic reactions in controlled, ultrafinely microporous polymeric materials with the prevention of recombination of the reaction products by their division into the two labyrinths. This together specific surface areas for reaction on the order of 10³-10⁴ square meters per gram, and with the possibility of readily controllable porewall surface characteristics of the two labyrinths.

Application: There are in fact two distinct biological systems in which Nature uses cubic phases (in unpolymerized form, of course) for exactly this purpose. Electron micrographs of the prolamellar body of plant etioplasts have revealed bicontinuous cubic phase microstructures (Gunning et al., Biochemistry of Chloroplasts Goodwin, Ed., Academic: London (1967) Vol. 2, pp. 655-676), and lipid extracts from these etioplasts have been shown to form cubic phases in vitro (Ruppert et al., Z. Pflyanzenphysiol. 90:101 (1978)). The prolamellar body develops into the thylakoid membrane of photosynthesis, which is again a continuous bilayer structure, with the stroma side acting as a cathode and the intrathylakoid side as an anode. Tien (Solution Behavior of Surfactants, Mittal et al., Eds. Plenum: New York (1982); Vol. 1, pp. 229-240) states that the chlorophyll dispersed in the lipid bilayer acts as a semiconductor, in that the absorption of light excites an electron to the conduction band and leaves a hole in the valence band. There are at least two reasons why the separation of the aqueous phase into two distinct compartments is important in natural photosynthesis: first, as well as providing an appropriate environment for the pigments, the bilayer acts as a barrier to prevent back-reactions; and second, with the two systems of accessory pigments located in distinct parts of the membrane, each electron/hole pair can be generated by two photons, thus providing an upgrading of photon energy. The endoplasmic reticulum, or ER, which is the site of the biosynthesis of many of the proteins needed by the cell, may also be a bicontinuous cubic phase, for certain electron micrographs indicate cubic order (Alberts et al., Molecular Biology of the Cell, pp. 335-339 (1983), Garland, N.Y.). Here the presence of two continuous aqueous labyrinths, one of which is continuous also with the exterior of the cell, creates a very large amount of surface area for reaction and continuity of ‘inner’ and ‘outer’ volumes to prevent saturation of concentration gradients which are the driving force for transmembrane transport. Clearly there is great potential impact in capturing and fixating such systems of high enzymatic activity and fundamental biological importance.

4) The microporous material exhibits in all cases a precisely controlled, reproducible and preselected morphology, because it is fabricated by the polymerization of a periodic liquid crystalline phase which is thermodynamic equilibrium state, in contrast to other membrane fabrication processes which are nonequilibrium processes.

Application: As is well-known in the industry, any microporous material which is formed through a nonequilibrium process is subject to variability and nonuniformity, and thus limitations such as block thickness, for example, due to the present material, the microstructure is determined at thermodynamic equilibrium, thus allowing uniformly microporous materials without size or shape limitations to be produced. As an example, the cubic phase consisting of 44.9 wt %, DDAB, 47.6% water, 7.0% styrene, 0.4% divinyl benzene (as cross-linker), and 0.1% AIBN as initiator has been partially polymerized in the author's laboratory by thermal initiation; the equilibrated phase was raised to 85° C., and within 90 minutes partial polymerization resulted; SAXS proved that the cubic structure was retained (the cubic phase, without initiator, is stable at 65° C.). If full polymerization by thermal initiation is possible, then such a process could produce uniform microporous materials of arbitrary size and shape.

5) Proteins; in particular enzymes, can be incorporated into the cubic phase bilayer and then fixated by the polymerization, thus creating a permanented reaction medium inheriting the precision of the present material, and maintaining to the highest possible extent the natural environment of the protein. This was illustrated in one of the experiments reported above. Many proteins and enzymes are specifically designed to function in a lipid bilayer, with hydrophilic and hydrophobic regions that match those of the natural bilayer. As shown by K. Larson and G. Lindblom (Larsson et al., Disp. Sci. Tech. 3:61 (1982)), a very hydrophobic wheat fraction, gliadin, can be dispersed in monoolein, and a bicontinuous cubic phase formed on the addition of water; in this case the protein is in the lipid regions of the cubic phase. Examples of other proteins and enzymes which can be incorporated into bicontinuous cubic phases, at thermodynamic equilibrium, have been reviewed (Ericsson et al., Biochim. Biophys Acta. 729:23 (1983)).

Application: Immobilized enzymes offer many advantages over enzymes in solution, including dramatically increased stability in many cases as well as higher activity and specificity, broad temperature and pH ranges, reusability, and fewer interferences from activators and inhibitors. to name a single example in the growing field of immobilized enzymes for medical assays, enzyme tests can distinguish between a myocardial infarction and a pulmonary embolism, while an EKG cannot. The present methods for immobilizin enzymes such as adsorption and covalent bonding have serious drawbacks. Absorbed enzymes easily desorb upon changes in pH, temperature, ionic strength, etc. The covalent bonding of enzymes usually involves harsh chemical conditions which seriously reduce enzymatic activity and cause significant losses of expensive enzymes. Recently a process has been developed for covalently bonding enzymes to collagen in such a way as to avoid exposing the enzyme to harsh chemistry (Coulet et al., Biochimie 62:543 (1980)). However, collagen is an extremely powerful platelet antagonist, activating fibrin and leading to immediate clotting, making it totally unsuitable for applications involving contact with blood. As shown above, enzymes can be immobilized in polymerized bicontinuous cubic phases with the enzyme continually protected in a natural lipid-water environment throughout the process.

6) The components can be chosen so that the material is biocompatible, opening up possibilities for use in controlled-release drug-delivery and other medical and biological applications that call for nontoxicity. It is known that many biological lipids from bicontinuous cubic phases, and many possibilities exist to modify such lipids to add polymerizable double or triple bonds to the tails, or to fix the structure using an aqueous-phase polymerization.

Application: Bicompatible materials of the type described are being investigated as polymerized drug-bearing cubic phases for controlled-release applications with high stability. The combination of the biocompatibility and entrapping properties of many cubic phases with the increased stability upon polymerization could lead to new delivery systems, and even the possibility of first-order drug release—release in response to physiological conditions—by incorporating proteins and enzymes, as described above, as biosensors.

### Polymeric Cubic add Other Liquid Crystalline Phases

While the primary emphasis of this chapter has been on polymerized liquid crystals, important insight into cubic phases and the driving forces behind their formation can be gained by comparing these with polymeric analogues, in particular with bicontinuous phases of cubic symmetry that occur in block copolymers and in systems containing water and a polymeric surfactant. There are two fundamental reasons why the observation of bicontinuous cubic phases in block copolymers is of tremendous value in helping to understand cubic phases in general: first, the applicability of statistical approaches, and the comparative simplicity of intermolecular interactions (summarized by a single Flory interaction parameter), make the theoretical treatment of block copolymer cubic phases (Anderson et al., Macromolecules, in press) much more straightforward than that of surfactant cubic phases; and second the solid nature and higher lattice parameters in the copolymer cubic phases make them readily amenable to electron microscopy (Thomas et al., Macromolecules 19(8):2197 (1986)).

The cubic microstructure that has in fact been observed in block copolymers is the #224 structure discussed above, with one of the blocks located in the two channels lying on the ‘inside’ of the surface, and the other block in the ‘matrix’ on the ‘outside’ of the surface, so that the surface itself describes the location of the junctions between the unlike blocks. In the polymer literature this structure has been referred to as the ‘ordered bicontinuous double-diamond’, or ‘OBDD’ structure. The structure occurs in medium-MW star diblock copolymers at higher arm numbers, and apparently also in linear diblocks at higher-MW (Hasegawa et al., Macromolecules 20:1651 (1987)), but always at compositions where the matrix component is between 62 and 74 vol. %. In early experiments, bicontinuity was indicated by vapor transport, and also by an order of magnitude increase in the storage modules over that of the cylindrical phase at the same composition but lower arm number. TEM tilt-series, together with SAXS measurements, taken at the University of Massachusetts at Amherst, have provided accurate and detailed data on the structure (Thomas et al., Macromolecules 19(8):2197 (1986)). In FIG. 16 is shown a split image, with electron microscopy data on the left half, and on the right half a computer simulation using the constant mean curvature dividing surface shown in FIG. 10A. The agreement is remarkable.

A theoretical treatment (Anderson et al., Macromolecules, in press) of the OBDD structure, employing the Random-Phase Approximation (RPA), yields accurate predictions of the lattice parameters from input data on the two blocks, and rationalizes the occurrence of the OBDD at compositions just below 74 vol. % as being due largely to a very low interfacial surface area for the model structure at these compositions, together with a mean curvature that is intermediate between lamellae and cylinders. One important conclusion from the theory is that the interface is very close to constant mean curvature, and this is supported by comparisons of the TEM data with simulations based on various interfacial shapes. However, care must be exercised in carrying over these ideas to the surfactant case, because in the small molecule case there is a higher penalty for variations in end-to-end distances for surfactant tails as compared to polymer chains. Nevertheless, the concepts of interfacial mean curvature, uniformity in stretch distances, and low interfacial areas apply in qualitatively similar ways in the two cases and appear to be the fundamental driving considerations for the occurrence of bicontinuous cubic phases in general.

And finally, a work should be said about cubic phases made from polymeric surfactants. Groundwork was laid by Kunitake et al. (Kunitake et al., J. Am. Chem. Soc. 103:5945 (1981)), who produced vesicles from polymeric surfacants. Very recently, polymeric surfactants of the ethoxylated alcohol type were shown to form cubic phases (Jahns et al., Coll. Polymer Sci. 265:304 (1987)). However, these authors were unaware of the notion of bicontinuity in cubic phases, and interpreted their results solely in terms of close-packed micelles. In particular they were unaware of the fact that low-MW ethoxylated alcohol surfactants (such as C₁₂E₆) form, in the same region of the phase diagram as their polymeric cubic phase, a bicontinuous cubic phase of the Ia3d type. With this knowledge in mind, it is quite possible that their polymeric cubic phase was indeed bicontinuous, but unfortunately the authors did little to characterize the phase. Since polymeric surfactants are far from ‘typical’ polymers, it is difficult to ascertain from first principles what the properties of such a phase should be, whether they should have mechanical properties reflective of glassy polymers or closer too those of liquid crystals, for example. An experimental complication is the fact that there are no cubic phases in the phase diagram for the monomeric surfactant. This example serves to remind us that the exact relationship between polymeric and polymerized bicontinuous cubic phases is as yet unknown, and many interest questions remain as to how far the analogy can be carried and whether or not there exists a continuum path between small molecule liquid crystalline and macromolecular bicontinuous states.

### A Related Microstructure

### Isotropic Bicontinuous Solutions in Surfactant-Solvent Systems

### The L₃ Phase

The structure of the isotopic L₃ phase observed in many surfactant-water or surfactant-water-oil systems is analyzed. It is pointed out that the L phase generally appears in equilibrium with a dilute solvent phase on one hand and a lamellar liquid-crystalline phase on the other. Irrespective of the detailed chemical nature of the stem, the one-phase region is remarkably narrow in one direction, indicating that the thermodynamic degrees of freedom are effectively reduced by one due to an internal constraint in the phase. In accordance with previous work it is argued that the basic structural unit in the L₃ phase is a surfactant bilayer. Furthermore we conclude that the L₃ phase appears when there is a spontaneous mean curvature towards the solvent at the polar/apolar interface. It is shown that for a system which has such a curvature towards the solvent, the surface formed by the bilayer midplane has a negative average Gaussian curvature <K>. By virtue of the Gauss-Bonnet theorem the bilayer under such circumstances has a multiply-connected structure. The conclusion is then that under conditions when there is a spontaneous mean curvature towards the solvent, it is possible to reach a low free energy state by forming multiply connected bilayer structures, as in many cubic phases, rather than planar bilayers. When interbilayer forces are weak the structure tends to be disordered, lea mg to an isotropic solution (L₃) rather than an ordered cubic structure.

To minimize local variations in curvature at the polar/apolar interface, we demonstrate that the midplane surface should be close to a minimal surface. We then show that a certain dimensionless group associated with a given periodic minimal surface has approximately the same value for all of the well-known isotopic minimal surfaces. Assuming a minimal midplane surface, we can then show that for a given thickness, a bilayer structure with a prescribed area-averaged mean curvature can only exist at a single volume-fraction. This explains the internal constraint in the L₃ phase, which is manifested in the narrow character of the L₃ phase. Applying the equations which express this constraint, and using results from a theory due to Cantor to account for the effect of water/head group interactions on water penetration, we present fits of these narrow Phase-existence regions to the theory, and rationalize the temperature dependence of the L₃ phases in a variety of nonionic surfactant systems.

The emerging picture of the L₃ phase is that the solution structure is characterized by a highly-connected bilayer, extending in three dimensions, thus appearing bicontinuous in, e.g., NMR self-diffusion experiments, and having an average mean curvature at the polar/apolar interface towards the solvent. The basic driving force forming an L₃ rather than a lamellar phase is thus not an entropy increase associated with forming finite lamellar patches, as previously suggested, but rather the opportunity to obtain an optimal curvature of the surfactant monolayer.

1. Introduction

Surfactant-water-oil system show an amazingly rich phase behavior, which is related to the fact that there s a large number of ways to divide space into polar and apolar regions with a given surface-to-volume ratio. Adjacent polar and apolar regions are separate, by a film rich in oriented surfactant molecules. The (oil+surfactant chains)/(water+head groups) volume ratio determines the area per polar group and the spontaneous (or ‘preferred’) curvature of the surfactant monolayer. The optimal aggregate structure in a particular case is determined to a large extent by general physical characteristics and systems which are chemically very different can show analogous phase behavior.

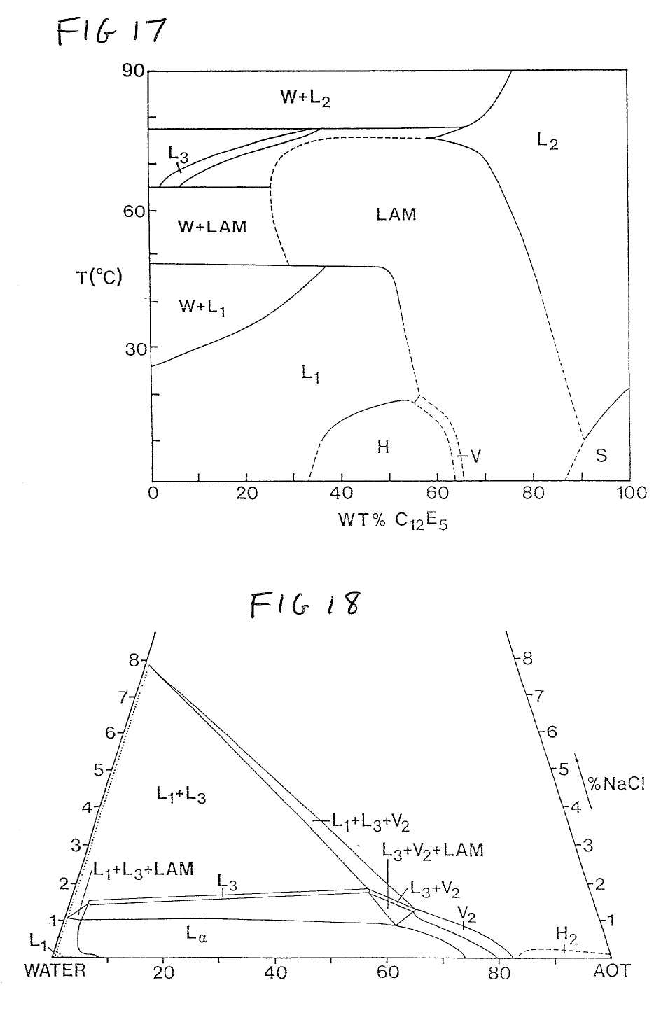

One example of a phase that shows the same characteristic behavior irrespective of the chemical details is the so called L₃ phase (sometimes called the ‘anomalous phase’). This phase has been observed in a number of binary nonionic surfactant water systems (Harusawa et al., Colloid & Polymer Sci. 252:613 (1979); Lang et al., J. Chem. Phys. 73:5849 (1980); Bostock et al., Colloid Interface Sci 73:368 (1980); Mitchell et al., J. Chem. Soc. Faraday Trans 1 79:975 (1983) and Persson et al., Colloid Interface Sci. 102:527 (1984)); a representative phase diagram is shown in FIG. 17. The same type of phase is found in some ionic surfactant/water systems in the presence of salt (Fontell, K. In “Colloidal Dispersions and Micellar Behaviour”; American Chemical Society; Washington D.C. (1975); ACS Symp. Ser. No. 0 p.270; Bellocq et al., Colloid interface Sci. 79:419 (1981); Ghosh et al., Colloid Interface Sci. 100:444 (1984) Ghosh et al., J.Phys. Chem. 91:4528 (1987)). An example from the early studies of Fontell (Fontell, K. In “Colloidal Dispersions and Micellar Behaviour”; American Chemical Society; Washington D.C. (1975); ACS Symp. Ser. No. 0 p.270) on the AOT-NaCl-H₂O system is shown in FIG. 18. In oil-water-surfactant systems L₃ phases can be found that are either rich in water or rich in oil (Kunieda et al., J. Disp. Sci. Techn. 3:233 (1982); Kahlweit et al., Angew. Chem. 24:654 (1985); Bellocq et al., in “Microemulsions:Structure and Dynamics” S.Briberg and P.Bothorel eds. CRC Press p.33 (1987); Olsson et al., J.Phys. Chem. 90:4083 (1986)). An example is shown in FIG. 19. The same type of phase has been identified also for dipolar (Laughlin R. G. Adv. Liq. Cryst. 3:99 (1978)) and zwitterionic (Marignan et al., J. Phys. Chem. 92:440 (1988)) surfactants, and with a triglyceride as oil (Kunieda et al., J. Phys. Chem. 92:185 (1988)).

The L₃ phase is an isotropic solution that is generally in equilibrium with both the dilute solution and a lamellar liquid crystalline phase. It is rather viscous, shows flow birefringence² and scatters light strongly (Lang et al., J. Chem. Phys. 73:849 (1980) and Fontell, K. In “Colloidal Dispersions and Micellar Behaviour”; American Chemical Society; Washington D.C. (1975); ACS Symp. Ser. No. 0 p.270), showing the presence of extended surfactant aggregates. As seen in FIGS. 17 and 18 the L₃ phase has a very narrow stability range, with practically only one degree of freedom rather than two as given by Gibbs' phase rule under the given circumstances.

The appearance of an L₃ phase is correlated with the presence of a lamellar phase, which strongly indicates that the aggregate structure is locally of a bilayer type. On the basis of detailed diffusion measurements it was suggested that the phase consists of disordered lamellae (Nilsson et al., J. Phys. Chem. 88:4764 (1984)). This qualitative conclusion was given a more quantitative formulation first by Miller and Ghosh (Miller et al., Langmuir 2:321 (1986)) and more recently by Cates et al. (Cates et al., Europhys. Lett. 5:73 (1988)). The latter paper contains a detailed model for the entropy increase on forming randomly oriented finite sheets from an ordered lamellar structure. Opposing the breakdown of the lamellar structure is the stiffness of the bilayer, which is assumed to have zero spontaneous curvature. However, it has been shown (Anderson et al., J. Chem. Phys., Submitted) that the area-averaged mean curvature in the mode of Cates et al. is moderately toward the solvent.

In the present paper we reanalyze the problems of the structure and occurrence of the L₃ phase by using differential geometry to describe structures of nonzero curvature. In this way we arrive at a suggested structure that provides a natural rationalization of the intriguing properties of the L₃ phase.

2. The Spontaneous Mean Curvature of the Surfactant Layers

An ideal surfactant is insoluble in both water and oil resulting in a self-association of the surfactant molecules, which in the first stage can be considered to lead to the formation of a monolayer film. Depending on the circumstances this film can curve towards the apolar side, or towards the polar side, or it can curve on the average towards neither. One of the most useful concepts for the qualitative understanding of phase equilibria in surfactant systems is based on the geometrical characterization of surfactant molecules suggested by Tartar (Tartar, H. V. J. phys. Chem. 59:1195 (1955)) and later developed by Tanfor (Tanford, C. “The hydrofobic effect” Wiley: New York (1973)) and by Israelachvili and coworkers (Israelachvili et al., J. Chem. Soc. Faraday Trans. 2 72:1525 (1976); and Israelachvili et al., Rev. Bikophys. 13:121 (1980)). The crucial dimensionless quantity is the v/la ratio formed by the molecular volume v, the molecular length l and the polar group cross-sectional area a. When v/la equals unity one has optimal conditions for a lamellar structure, while for v/la>1 the surfactant film prefers to curve towards the water, while for v/la<1 the optimal curvature is in the other direction. Although extremely useful for qualitative arguments, it is difficult to use this approach for more quantitative discussions, in particular since the area per polar group a depends on composition and temperature in a complex way. A concept related to the v/la ratio that has more general character is the notion of the spontaneous curvature, H0, of the surfactant monolayer. This was first introduced for amphiphile systems by Helfrich (Helfrich, W. Z., Naturforsch. 28c:693 (1973 )) when discussing phospholipid bilayer systems. The virtue, and the weakness, of this approach is that we can introduce a certain H₀ while leaving the question of the molecule source of the particular value unanswered.

For an ionic double chain surfactant, as for example AOT of FIG. 18, v/la is often close to unity and a lamellar phase is stable over a wide concentration range. At high water contents, where the electrostatic interactions are strongest (Jönsson et al., Colloid Interface Sci. 80:482 (1981) and Jönsson et al., J. Phys. Chem. 92:338 (1987)) the spontaneous curvature of the monolayer is towards the apolar region, i.e. H₀ is positive, while at low water contents will less influence from electrostatic interactions the spontaneous curvature is negative. This leads to the formation of a bicontinuous (Lindblom et al., Biophys. Chem. 6:167 (1977)) cubic and ultimately to a reversed hexagonal phase on increasing the surfactant content. When salt is added to this system the electrostatic contribution to the curvature free energy will decrease, driving H₀ towards negative values. For a given concentration of salt in the aqueous region, the effect on W of the salt will be largest at high water contents, where the electrostatic contributions are largest. With more than 1.5% NaCl in the system the spontaneous curvature is expected to be towards the water over the whole stability range of the L₃ phase shown in FIG. 18.

For nonionic surfactants based oligomeric ethylene oxide (EO) chains as the polar group, the phase behavior is strongly influenced by temperature, so that the higher the temperature the less hydrophilic is the surfactant. As the hydrophilicity, and thus the expected water penetration, decreases with increasing temperature, we expect that the spontaneous mean curvature should decrease. The details of the phase equilibria depend on the number of carbons in the chain and on the number of EO groups (Mitchell et al., J. Chem. Soc. Faraday Trans 1 79:975 (1983)), but the general pattern is the same in systems showing L₃ phases. At lower T-values there is a region where the lamellar phase is an equilibrium with a dilute solution. At higher T an L₃ phase intervenes in such a way that at the low-T end the L₃ phase has a higher water content than at the high-temperature end. For C12E₅ in FIG. 17 one can follow the full behavior from micellar solutions and normal hexagonal liquid crystals at low temperatures, to the appearance of a lower critical point (cloud point), and the formation of a lamellar phase on increasing T; then at still higher T, we find an L₃ phase and in some systems such as C16E₄, a bicontinuous inverted cubic phase (Mitchell et al., J. Chem. Soc. Faraday Trans 1 79:975 (1983)), and finally an L₃ phase. Although it is not necessarily true in these systems that the L₃ phase consists of inverted micelles, all of these progressions appear to be consistent with a change in mean curvature from toward oil in the normal structures at low T, to toward water in the inverted structures at high T, supporting the hypothesis that the mean curvature is toward water in the binary L₃ phase.

Studies of microemulsion systems, like the one shown in FIG. 19, further substantiate this conclusion concerning the change in the sign of H0. At high water contents (say α=0.1), there are at low temperatures slightly swollen normal micelles in equilibrium with excess oil. On the increasing T one enters the one-phase microemulsion channel. At the lower end there are highly swollen micelles (Olsson et al., J.Phys.Chem. 90:4083 (1986) and Olsson et al., J.Phys.Chem., In press (1988)). At the high temperature end, H₀−¹ is larger than the radius of the largest monodispersed spheres that can be formed and a dramatic change in aggregate shape and size occurs (Olsson et al., J.Phys.Chem. 90:4083 (1986) and Olsson et al., J.Phys.Chem., In press (1988)). At still higher T a lamellar phase is formed, and at approximately 45-50C in the case of C₁₂E₅, we reach the condition H₀≈0, since there the lamellar phase shows maximum stability. By further increasing T, we expect the spontaneous mean curvature H₀ to become negative, and thee one finds the L₃ phase. This branch of the L₃ phase connects to the L₃ phase in the binary system, making it plausible that H₀<0 also for the binary L₃ phase.

The conclusions about the H₀ values in the ternary system are given further support by the observation that at high α values, where oil is the dominating medium, the behavior is reversed. One finds water droplets in oil at high T, then a lamellar phase and finally an L₃ phase on lowering the temperature. Thus studies at high and low α values give the same conclusions concerning the spontaneous curvature of the surfactant film, namely that the mean curvature is toward the more abundant solvent. This ‘criss-cross’ pattern, in which the L₃ phase crosses the main microemulsion channel, showing an opposite temperature dependence, is observed in other similar systems. In the present paper this is interpreted in terms of a fundamental difference in the relationship between the spontaneous mean curvature H0 and the oil/(oil+water) ratio α for the monolayer—microemulsion—and bilayer—L₃ phase—microstructures. In the monolayer case, the mean curvature is toward the less abundant solvent (Anderson, D., Ph.D. Thesis, University of Minnesota (1986)) (whether discrete or continuous), whereas in the case of a bicontinuous bilayer structure the mean curvature is toward the more abundant solvent (Anderson et al., J. Chem. Phys., Submitted).

The phenomenological conditions under which the L₃ phase is observed suggests the following conjecture: An L₃ phase is formed when the locally preferred structure is a bilayer, but when the surfactant monolayer has a spontaneous curvature towards the abundant solvent. To analyze the consequences of this conjecture we make use of some of the recent advances in the application of differential geometry to the study of surfactant aggregates structures (Anderson et al., J. Chem. Phys., Submitted; Helfrich, W. Z., Naturforsch. 28c:693 (1973); Anderson, D., Ph.D. Thesis, University of Minnesota (1986); Hyde et al., Z. Kristallogr. 168:213 (1984); Anderson et al., Proc. Natl. Acad. USA, In press (1988)).

3. Curvature Free Energies

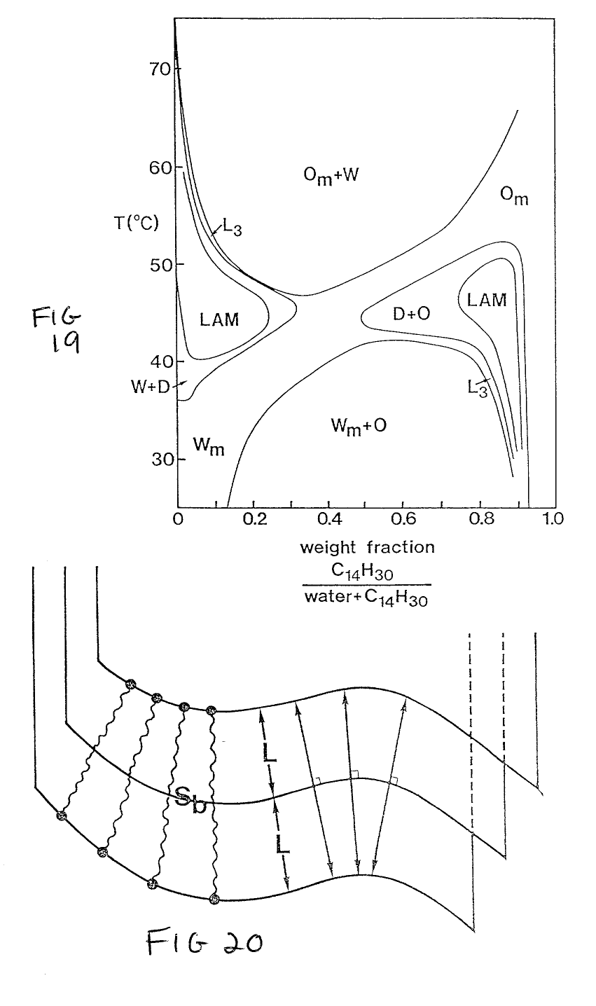

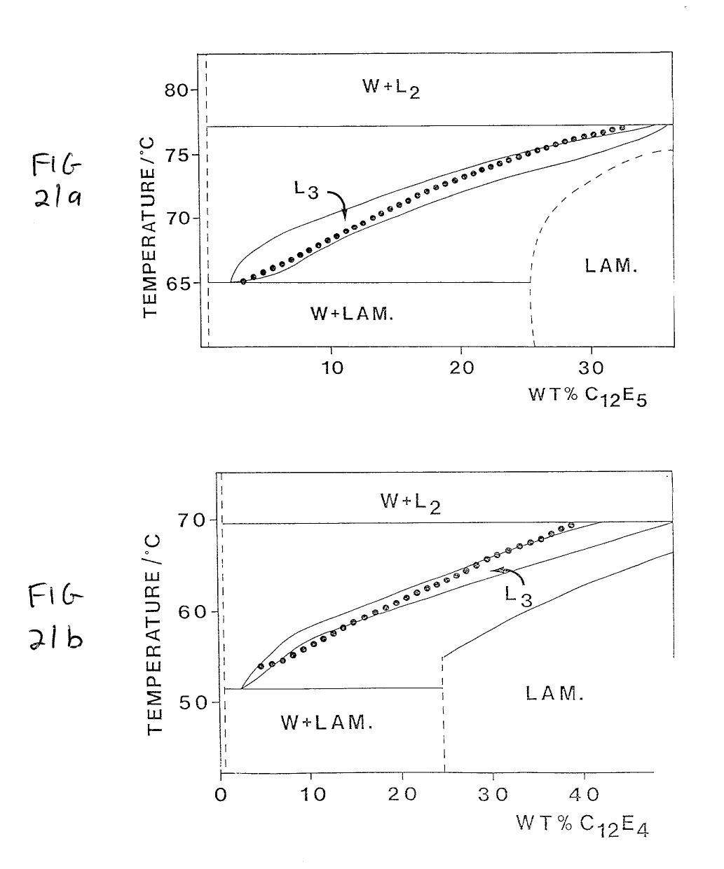

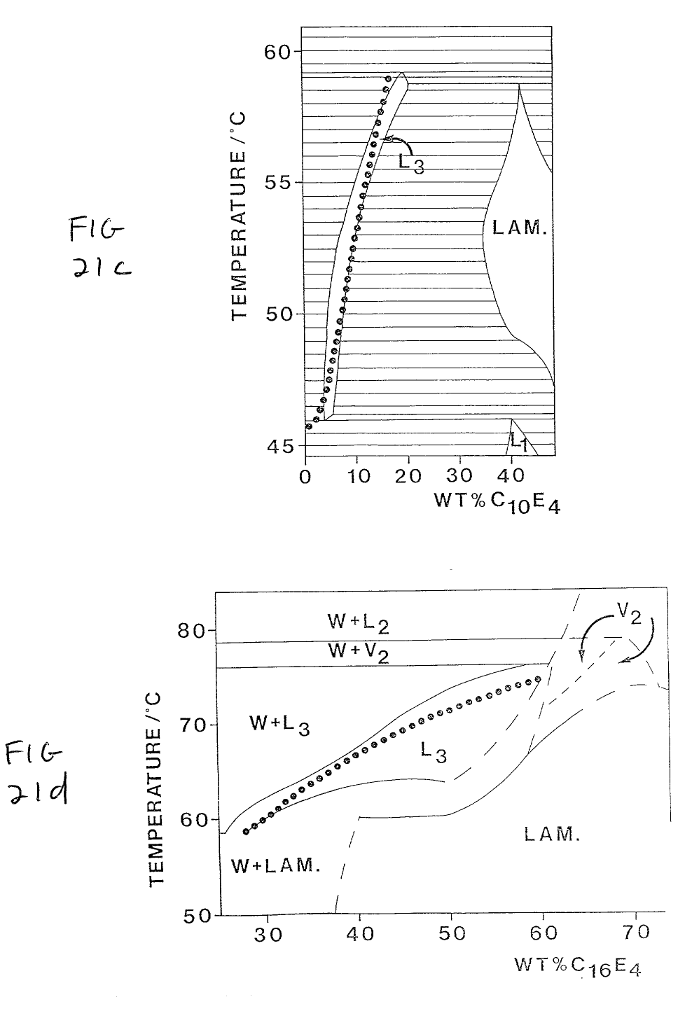

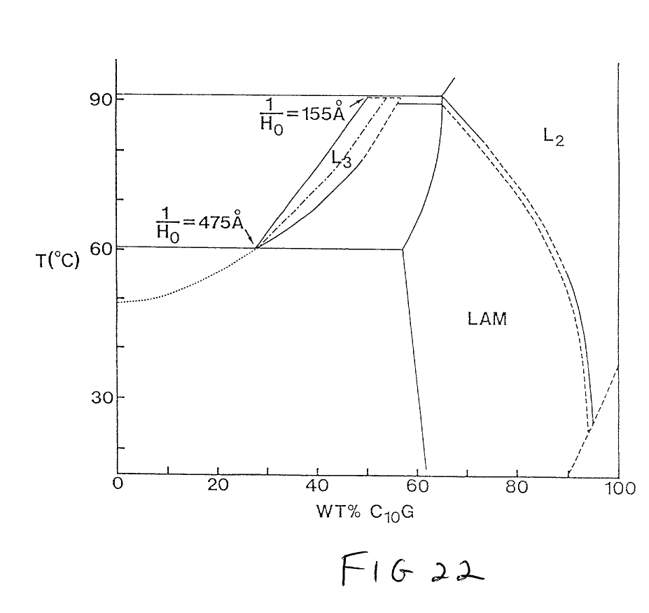

For a surfactant bilayer one can identify three approximately parallel surfaces, one at the midplate of the bilayer, here denoted the base surface, and two parallel surfaces a distance on either side of the base surface describing the polar/apolar interface (see FIG. 20). We want to assign the curvature energy of the surfactant monolayer in relation to the headgroup plane because interactions are strongest in this region. Let HL denote the pointwise mean curvature on the parallel surface. The area-weighted average mean curvature <H_{L}> over the two displaced surfaces is (Anderson et al., J. Chem. Phys., Submitted).

<H_{L}>=L<K>/(1+L²<K>) (1)

where <K> is the average Gaussian curvature of the base surface. Note that this average <H_{L}> is independent of the mean curvature H_{b}. Normally ¦L²<K>¦<1 (see Appendix), so that if we require <H_{L}> to be negative in accordance with the conjecture for the L₃ phase presented above, the base surface should have a negative Gaussian curvature. By virtue of the Gauss-Bonnet theorem (Struik, D. J. “Lectures on Classical Differential Geometry” Addison & Wesley, Cambridge, Mass. (1980)).

<K>=2πX_{E} (2)

where X_{E} is the Euler characteristic of the surface which is related to the connectivity of the surface. Through eq.(2) a negative <K> necessarily implies that the surface is highly connected. The larger the value of −<H_{L}>, the larger is −<K> and thus X_{E} and the more connected is the surface per unit volume. Examples of such highly-connected surfaces are periodic minimal surfaces (Schwartz, H. “Gesammelte Mathematische Abhandlungen”, Vrlag-Springer, Berlin (1890)). The more common minimal surfaces D,P and the gyroid have, for example, Euler characteristics X_{E}^{U} of −2,−4 and −8 per unit cell, respectively. This shows by a straightforward, but somewhat esoteric, to geometrical argument that a bilayer structure with negative average mean curvature towards the solvent can only be formed through building up a highly-connected surfactant aggregate. The sole restriction is that branch points with three or four monolayer films meeting are not allowed. For systems with H₀<0 but H₀L¦<<1, branch points are clearly energetically unfavorable.

In an expansion to second order the curvature free energy area density, g_{c}, is

g_{c}=K_{B}(H_{L}−H₀)² (3)

where K_{B} is the elastic bending constant. The total curvature free energy G_{c}, per area A is then

G_{c}=K_{B}<(H_{L}−H₀)²> (4)

where the area A includes both of the parallel surfaces. To minimize G_{c} it is clearly advantageous to have H_{L} close to H₀ not only on average but also at each point. This latter condition is difficult to satisfy for two parallel surfaces. In the Appendix we show that the base surface that gives a minimum in G_{c} must be a minimal surface, i.e., the mean curvature H_{b} of the base surface is zero. The reason for this is that one has the optimal homogeneity between the two parallel surfaces in such a case.

We thus find that the bilayer midplane in the L phase is close to a minimal surface. To obtain more quantitative relations, we begin by dividing the structure into cubes of edge length a. This characteristic length a is chosen so that the portion of the base surface enclosed in a cube is on the average of Euler characteristic X_{E}^{U} approximately equal to −4; we will see that it will cancel out in the final result, so we do not need a more precise definition here. The Euler characteristic per volume V is

X_{E}/V=X_{E}^{U}/a³ (5)

For a cubic system a would be the lattice parameter. The area A₀ of the base surface is similarly given by the product of the surface area ça² of the characteristic unit times the number of units

A₀=ça²V/a³ (6)

where the dimensionless constant ç is given by the particular structure. The volume fraction Φ_{B} of bilayer is, using Steiner's equation with H_{b}=0

Φ_{B}=2A₀L(1+<K>L²/3)V (7)

Solving eqs. (1,2,5-7) provides a relation between the volume fraction and the average mean curvature

$${φ}_{B}^{2} = \frac{2 {C}^{3}}{π {/}_{E}^{U}} L < {H}_{L} > \frac{{( 3 - 2 L < {H}_{L} > )}^{2}}{9 {( 1 - L < {H}_{L} > )}^{3}} \qquad ( 8 )$$

The factor −2ç³/(πX_{E}^{U}) depends on the particular structure. However a reference to periodic minimal surfaces shows that although these different surfaces have different Euler characteristics and different values for the constant ç the dimensionless group 2å³/X_{E}^{U}) is remarkably insensitive to the particular structure as illustrated in Table 1, except for deviations at highly negative Euler characteristics, which are physically less realistic (the largest value of −X_{E}^{U} for any known binary cubic phase is 8). Below we will set −2ç³/πX_{E}^{U})=2.2.

TABLE 1
Values of the dimensionless group −2ç³/πX_{E}^{U}) for minimal
surfaces of cubic symmetry whose areas are known. Surfaces are named as
in ref. 30. The space group listed is that for a cubic phase with the
minimal surface forming the midplane of a bilayer, except in those cases
indicated by an asterisk (*), which cannot support a symmetric bilayer (the
areas in these cases were computed numerically in ref. 30). The Euler
characteristic per unit cell X_{E}^{U} and the surface area per unit cell (with a
lattice parameter of unity) ç vary considerably from surface to
surface, while the dimensionless group- 2ç³/(πX_{E}^{U}) remains quite
constant for small values of −X_{E}^{U}. Cubic phases corresponding to
the surfaces above the dotted line have been reported in experiments.

| Surface | Space Group | X_{EU} | ç | −2ç³/πX₃^{U}) |
|---|---|---|---|---|
| D | Pn3m | −2 | 1.919 | 2.249 |
| P | Im3m | −4 | 2.345 | 2.053 |
| G | Ia3d | −8 | 3.091 | 2.350 |
| I-WP | Im3m* | −12 | 3.466 | 2.210 |
| C(P) | Im3m | −16 | 3.510 | 1.721 |
| F-RD | Fm3m* | −40 | 4.740 | 1.700 |

Within this approximation, eq.(8) shows that for a given structural unit there exists a unique relation between the volume fraction of bilayer and the average mean curvature over the displaced surfaces. If we require that <H_{L}>=H₀, eq.(8) imposes an internal constraint and the formal number of degrees of freedom is reduced by one and eq. (8) is changed to

$$\begin{matrix}{φ}_{B}^{2} = 2.2 L ( - {H}_{0} ) & \frac{{( 3 - 2 {LH}_{0} )}^{2}}{9 ( 1 - {LH}_{0} )}\end{matrix} \qquad ( \text{8a} )$$

≈2.2L(−H₀) (8b)

Thus by analyzing curvature free energies of a bilayer aggregate we have arrived at the remarkable result that when there is a spontaneous mean curvature towards the solvent, in the optimal structure the bilayer midplane forms a highly-connected surface at a distant optimal volume fraction of bilayer that is determined by the dimensionless product H₀L of the spontaneous curvature and the bilayer half-width.

It is important to point out that the result given in equation (8b) is not sensitive to the assumption that the bilayer is of constant thickness. As discussed in ref. 33 in the context of bicontinuous cubic phases, an alternative description of the polar/apolar interface is in terms of surfaces of constant mean curvature H_{L}, which show a variation in the distance from the minimal surfaces related to the Schwarz “D” or “Diamond” minimal surface, is approximately 7% of the average distance <L>, whereas the variation of mean curvature over the parallel surfaces considerably larger than this (not surprisingly, since mean curvature is second-order derivative property). We now derive an approximate formula analogous to equation (8b) for this particular family of constant-mean curvature models, to demonstrate that, at least for the case of structures with the Schwarz “D” minimal surface as the base surface, the result in equation (8b) is the same for the constant-mean-curvature interface as for the parallel surface interface.

The slope of the volume fraction vrs. mean curvature plot for the “D” family of constant-mean-curvature surfaces was estimated accurately in ref. 30 yielding Φ_{B}=−0.55928 h+. . . , where h=Ha is the mean curvature made dimensionless by multiplying with the lattice parameter. Since we will only be concerned with the highest order terms here, we can write an approximate formula for the relation between Φ_{B} and the average length <L>, as Φ_{B}=2<L>Aₘ/a +. . . , where Aₘ is the area of the minimal surface when a=1, which has the value Aₘ=−1.918893 . . . for the “D” surface. Multiplying these two equations gives ΦB²=−2.1464H<L>. This is very close to the result for to the family of surfaces parallel to the “D” minimal surface: Φ_{B}²=−2.24906 <H_{L}>L. Presently there has been no publication of a calculation of a aperiodic minimal surface, so there is no way to check whether or not we are correct in our assumption that these results for periodic minimal and constant-mean-curvature surfaces hold, at least approximately, for aperiodic analogues.

The bilayer volume fraction Φ_{B} is in general greater than the surfactant volume fraction Φ_{S} in the L₃ phase, because of the penetration of solvent into the bilayer. We define Φ_{SB} to be the volume fraction of surfactant in the bilayer region, that is, in the region between the two displaced surfaces:

φS=φSBφB (9)

The theory of Cantor (Cantor, R., Macromolecules 14:1186 (1981)) provides an estimate of Φ_{SB}. In the case of binary surfactant/solvent L₃ phase—particularly where the surfactant is closely related to diblock copolymers, as in the case of an ethoxylated alcohol surfactant—the melt/semidilute interface case treated by Cantor applies, and equation (47) of that paper implies that:

φSB=φJ/(φJ(1−f)+f), (10a)

where

φJ=c′(½−χ)−⅗T−⅖ (10b)

where φJ is the volume fraction of surfactant in the polar region, and ƒ is the volume fraction of the polar (EO) portion within the surfactant molecule. We have combined into a single constant c′ all of the numerical constants and those factors which have a lesser temperature dependence. In the case of ethoxylated alcohol surfactants, the interaction parameter X (not to be confused with an Euler characteristic!) between the water and ethylene oxide groups is known to be a strong function of temperature (Kjellander et al., Chem. Soc. Faraday Trans. 1 77:2053 1981)). It should be noted that if the chain stretching contribution to the free energy in the theory of Cantor is replaced by a term of the functional form (L_{I}-L₁₀)², which might be more appropriate for low-MW polar groups, the exponent of the term containing X remains between −⅔ and −½.

We approximate the temperature dependence of H₀ by retaining only the lowest order term in the Taylor series expansion, thus −H₀=c(T-T120). (The sign conventions in this formula must be changed for the case where the solvent is apolar). We do this on first principles, but it should be noted that the theory of Cantor also predicts a nearly-linear dependence of H₀ on temperature, at least in the case where both polar and apolar excess solvents exist. Equation (17) in ref. 36 shows that H₀ is a multiple of Q₂, which is linear in X; the other temperature dependencies in that expression are smaller, at least in the cases of most interest here where the temperature dependence of X is significant. The constant c^{½} will be combined with the factor (2.2 L)^{½} to yield a final constant c. The value of c′ must for the present be treated as a fitting parameter because the value of the bare surface tension X₁ in the theory of Cantor is unknown, but also because of the approximations involved in that theory and the present theory. Combining equations (8)-(10), the final expression for the optimal volume fraction of surfactant, at which <H_{L}>=H₀, is then:

φS=c(T-T₀)^{½}φJ/(φJ(1−ƒ),

where

φJ=c′(½−χ)^{-⅗}T^{-⅖} (11).

In this expression we have left out the correction term (3−2LH₀)/9(1−LH₀)^{3/2}, which is very close to unity whenever ¦LH₀¦<<1, this being the case at sufficiently low volume fractions. Furthermore, this correction term has a different functional form when constant mean curvature interfaces are assumed instead of constant width interfaces, so we choose to ignore this factor and use the first order term, i.e. eq. (8b), which is the same in the two cases.

From the point of view of demonstrating a good fit of experimental data using a small number of fitting parameters, it is unfortunate that the conversion of φ_{J} to φ_{SB} in equation (10a) means that c and c′ cannot be combined into a single fitting parameter, reducing the number of fitting parameters from 3 to 2. However, we have found, not surprisingly, that the final matches of experimental phase boundaries are very insensitive to the value of c′, and to a very large extent it is simply the product of c′ and c that determines the final results. We have in all cases taken c′ to be unity, but equally good results can be obtained with c′=½, for example. The two important parameters T₀ and c are fit to experimental data; for many polar groups, the temperature dependence of X is known from independent experiments.

4. Interpretation of the Experimental Phase Diagrams

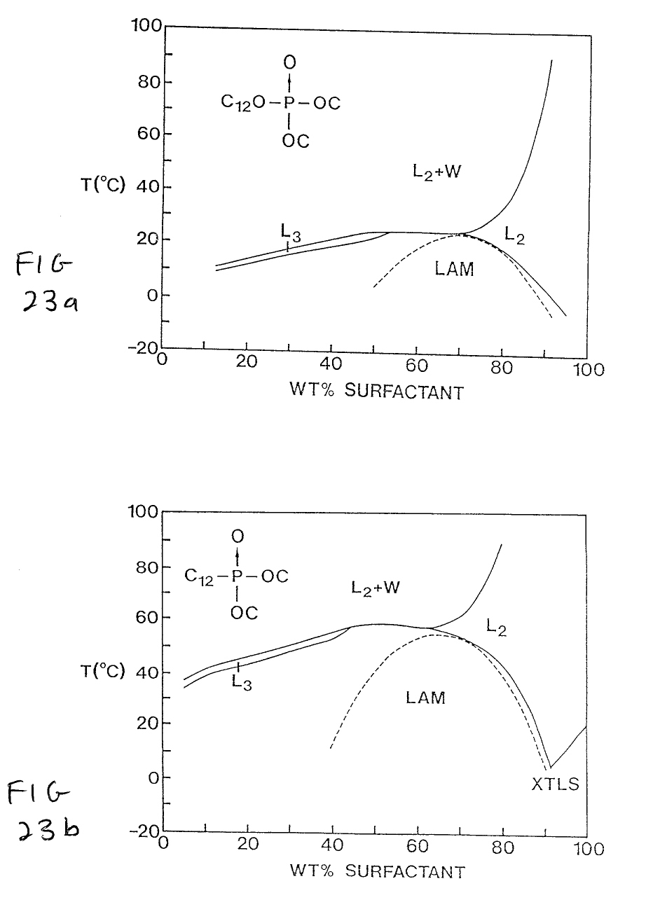

We now apply equation (11) to the location of the L₃ phase in those phase diagrams for nonionic surfactant/water systems tabulated by Sjoblom et al.38 which contain an L₃ region, as well as for one L₃ phase region in an ionic surfactant/water system. We begin with the ethoxylated alcohol/water systems. Kjellander and Florin (Kjellander et al., Chem. Soc. Faraday Trans. 1 77:2053 (1981)) have estimated the interaction parameters for the water/ethylene oxide interaction at three temperatures, namely 35, 45, and 69.5 C. By differencing their data, they estimated the enthalpic and entropic contributions to the interaction to be roughly −1460 cal mol−¹ and 5 cal mol−¹K−¹, respectively, at 40 C. For all of the cases shown in FIG. 21 we have used the expression X=2.876−483.5/T to obtain a fit of the data, which corresponds to enthalpic and entropic contributions of −1676 cal mol−¹ and 5.72 cal mol−¹K−¹, respectively. The values of φ_{J} resulting with this expression and the above formulae are in accord with standard estimates for the amount of water in the interfacial region, namely between about 2 and 7 water molecules per EO group, for temperatures below 70 C.

The fits for C₁₂E₅,C₁₂E₄,C₁₀E₄ and C₁₆E₄ are shown in FIGS. 21A-D, and the values for T₀ obtained from the fits are given in Table 2, which also includes the cloud point temperatures, T_{cp}, for comparison. In general for CₙEₘ surfactants, one would expect T₀, the temperature at which the spontaneous mean curvature in the binary system is zero, to increase with increasing m, because an increase in temperature acts to decrease the amount of water in the ethylene oxide regions (that is, increasing X causes an increase in Φ_{SB} by equation (10)), and thus counteract the increase in curvature toward hydrocarbon due to increased steric repulsion from more ethylene oxide groups. Similarly, T₀ should be expected to decrease with increasing n. These trends are observed except for the case of C₁₂E₄.

TABLE 2
Cloud point temperatures T_{cp}, and values of T₀ (estimated
temperature where the spontaneous mean curvature H₀ passes
through zero in the binary system), for the four ethoxylated alcohols
known to form L₃ phases. The entries are listed in order of
increasing HLB, defined as³⁸ the weight fraction of the
ethylene oxide portion of the molecule, multiplied by 20.
Intuitive arguments suggest at T0 should increase with increasing HLB,
because lower water penetration--and thus higher
temperatures--are required to reach the same balanced state
for more hydrophilic surfactants.

| Surfactant | HLB | T0 | Tcp |
|---|---|---|---|
| C₁₆E₄ | 9.2 | 35.0 | — |
| C₁₂E₄ | 10.7 | 53.5 | 5 |
| C₁₀E₄ | 11.6 | 45.3 | 20 |
| C₁₂E₅ | 11.7 | 64.5 | 26 |

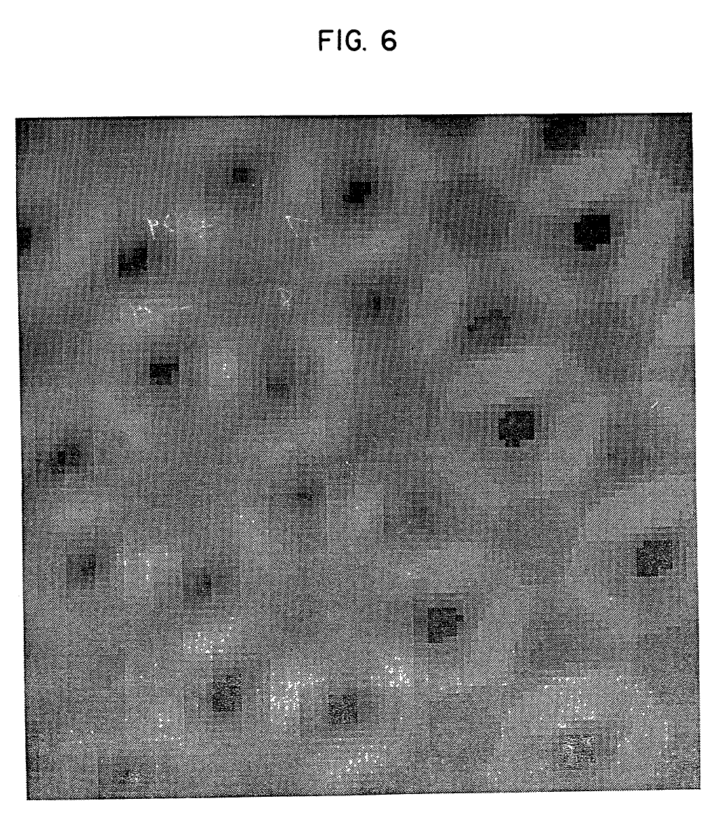

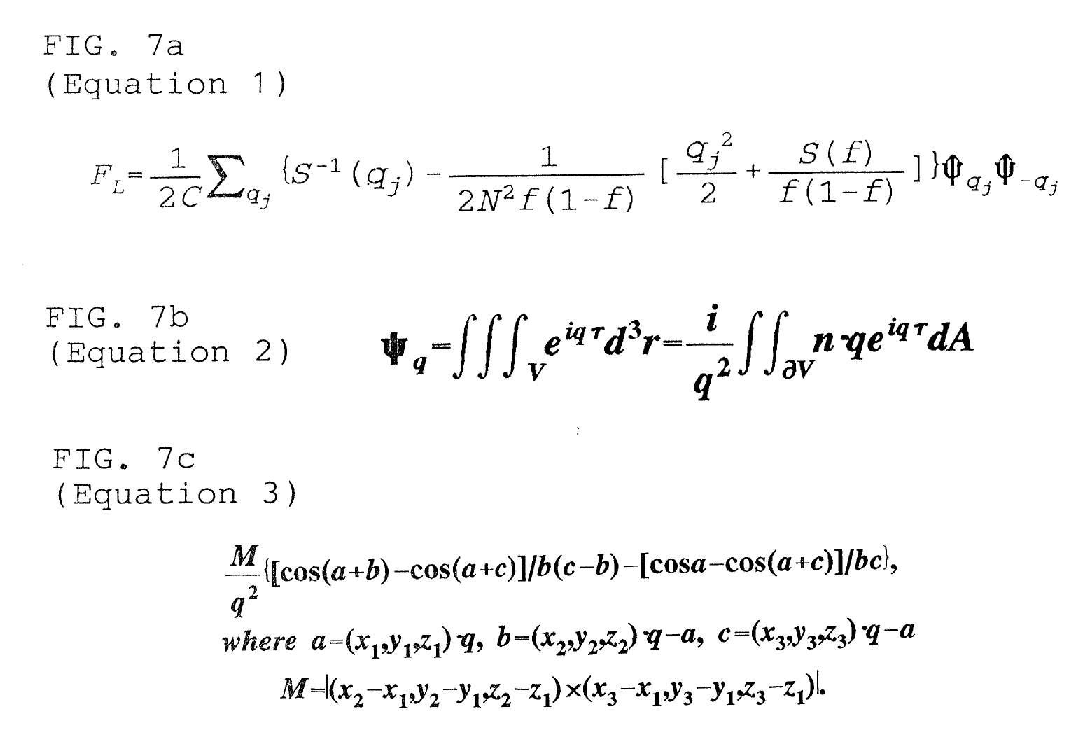

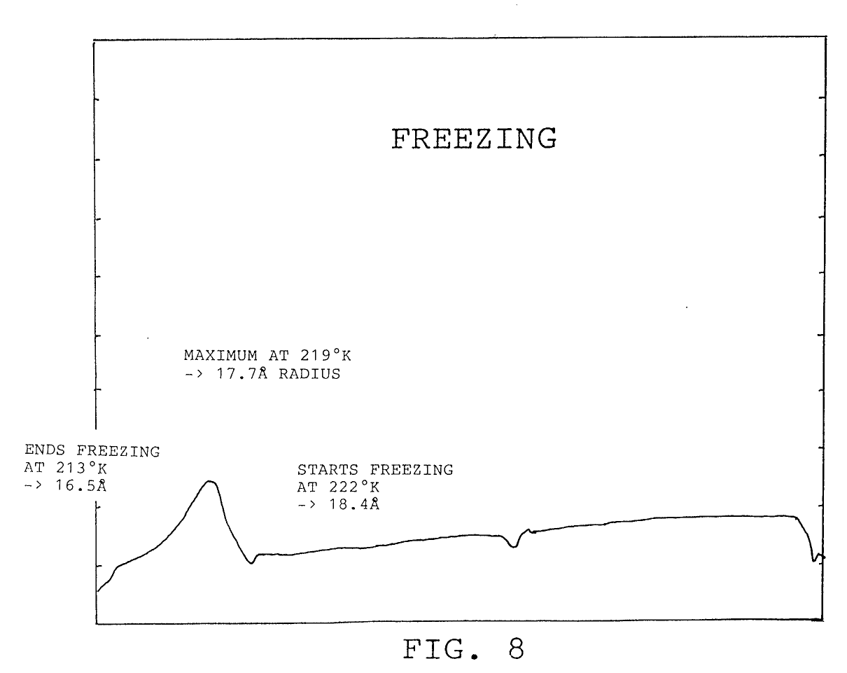

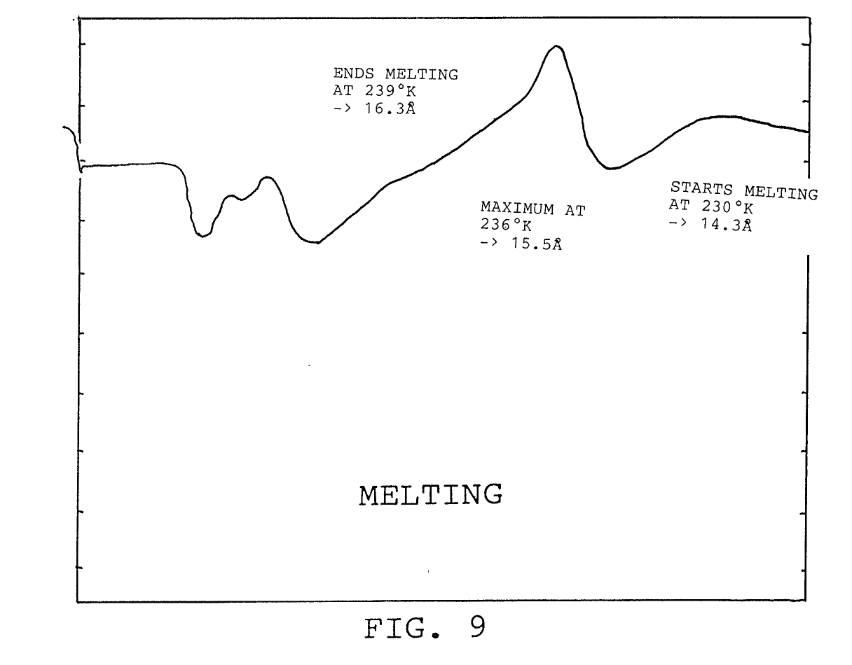

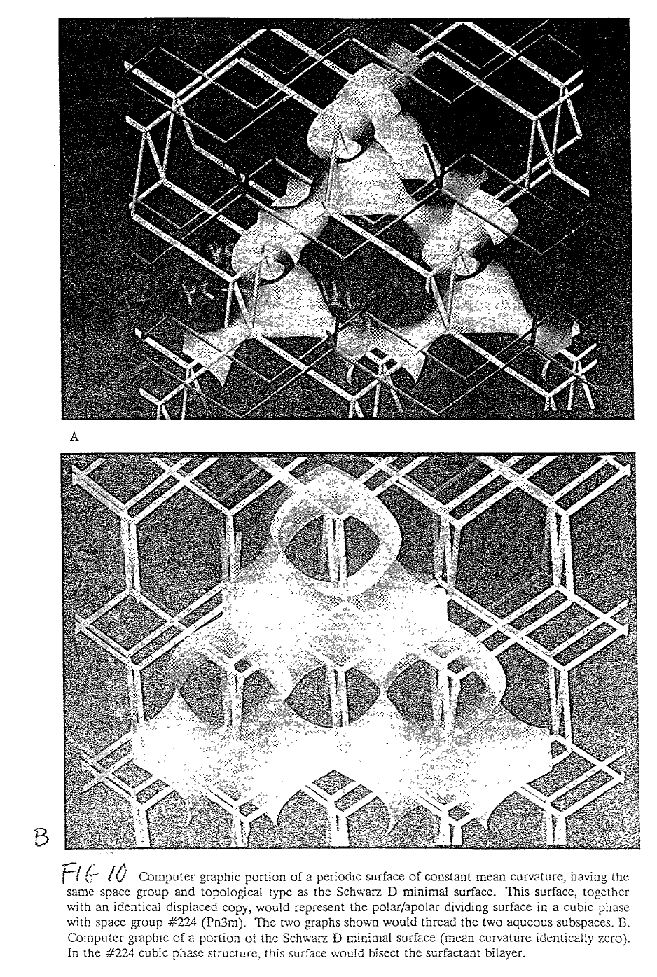

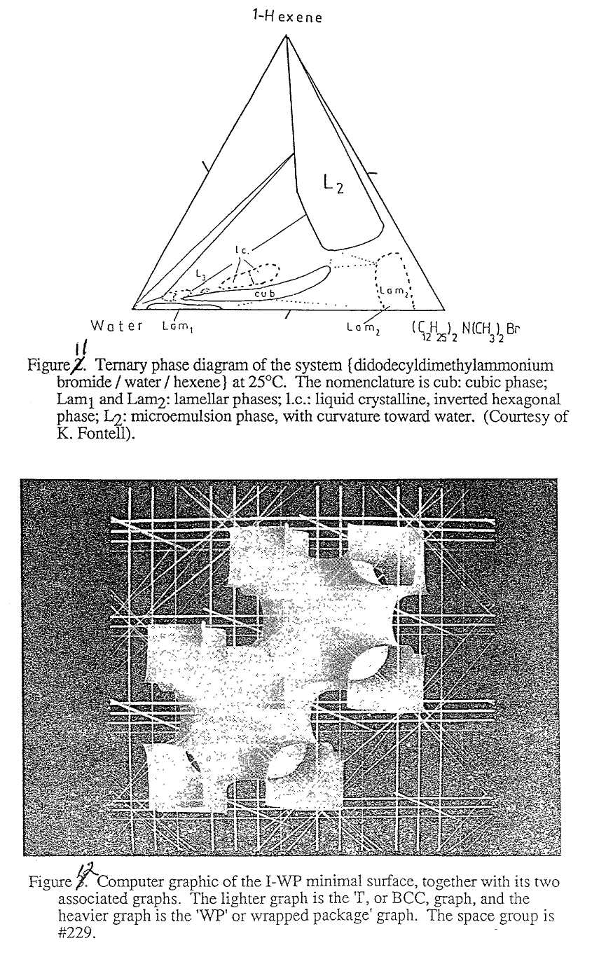

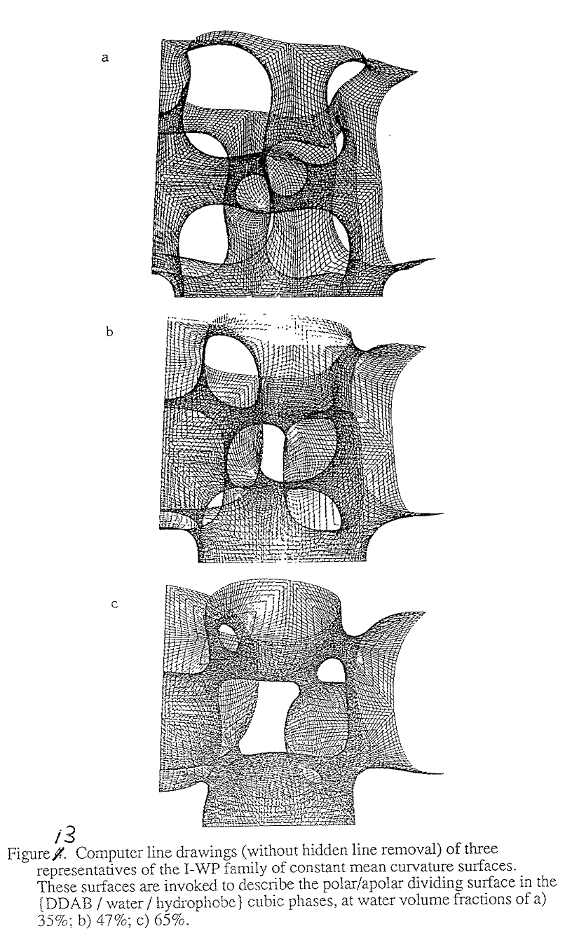

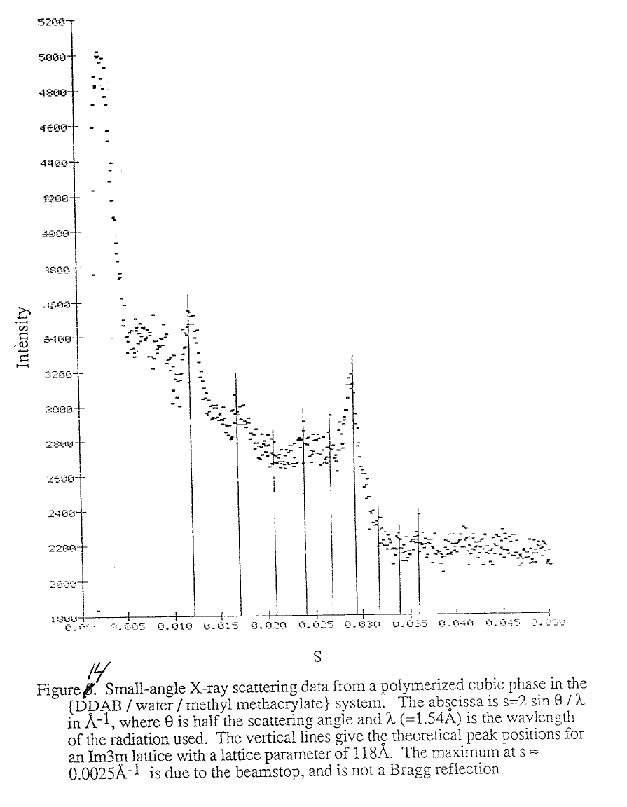

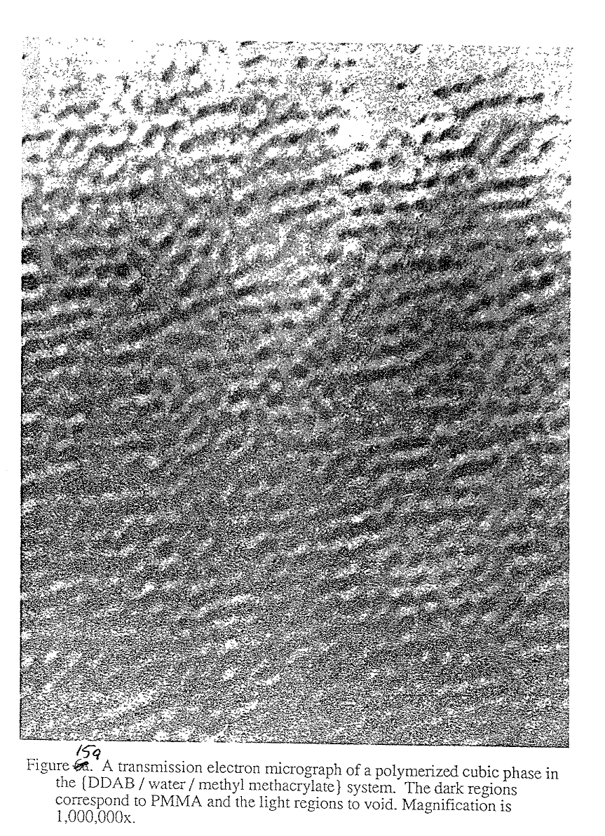

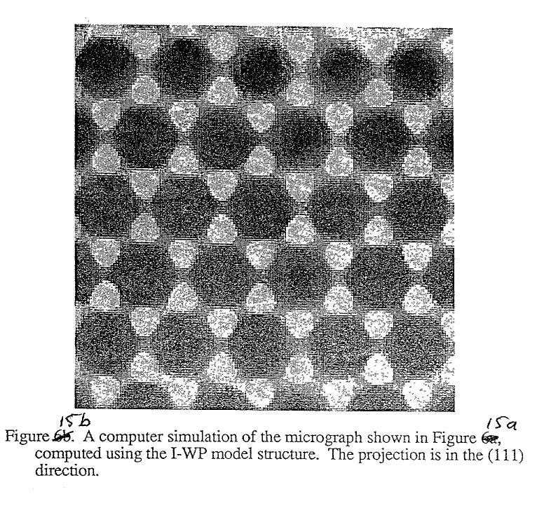

In the case of 1-O-decylglycerol (FIG. 6), the fit was obtained by assuming that the temperature dependence of X was negligible (Φ_{J}=constant for all T). In related monoglycerides, for example, it is known that the temperature dependence of the water/polar interaction is fairly weak, and that the phase behavior can be understood at lease qualitatively in terms of increasing chain disorder with increasing temperature (Larsson et al., N. Chem. Phys. Lipids 27:321 (1980)). This example illustrates the fact that, in the present theory, weakly temperature-dependent interactions will lead to a T vrs. Φ_{S} curve that is concave upward, whereas interactions that become strongly unfavorable at higher temperatures can lead to a curve that is convex. Further evidence of the lower temperature sensitivity in the C₁₀-glycerol system is in the much wider temperature range over which the surfactant concentration changes significantly: over 30 C. for the C₁₀-glycerol system, compared to roughly 15 C. for the CₙEₘ systems.

In this respect the phosphoryl surfactant systems containing L₃ phase regions are intermediate (FIG. 23). We have not attempted a curve fit with these systems because of because of the lack of data on the temperature dependence of X with these polar groups. However, this dependence appears to be non-negligible both from the lack of concavity of the L₃ phase region, and from the fairly narrow temperature ranges (roughly 20 C. in both cases) over which significant changes in ΦS occur.

In the simplest case for which the temperature dependence of the head group/water interaction appears to be least, namely C₁₀-glycerol, we have estimated, from our fit of theory to data (FIG. 22), a rough formula for the characteristic length of which we believe gives the correct order of magnitude for the length with composition. This formula gives a monotonic increase in a with decreasing concentration, from about 140 Å at φS=0.5 to about 230 Å at φS=0.27. In general for all the systems studied, the smallest curvatures and largest characteristic lengths are deduced to occur at the smallest surfactant concentrations. For the CₙEₘ systems, which reach to much lower values of Φ_{S}, it is more difficult to estimate the characteristic length because of the more complicated temperature dependencies, but it appears from order of magnitude estimates that this length could reach over 1,000 Å at the lowest concentrations. This is qualitatively in agreement with the observation of more rapid NMR relaxation (Nilsson et al., J. Phys. Chem. 88:4764 (1984)) and stronger light-scattering in this end of the L₃ phase.

For the AOT system in FIG. 18 the salt concentration in the aqueous regions of the L₃ phase increases as the bilayer volume fraction increases. Since the electrostatic forces, whose importance is decreased by the salt, favor a curvature towards the apolar region, H0 decreases—becomes more negative—with increasing Φ in qualitative agreement with eq. (8). The electrostatic effects are amenable to a quantitative analysis using Poisson-Biltzmann approach (Jönsson et al. Colloid Interface Sci. 80:482 (1981); Jönsson et al., J. Phys. Chem. 92:338 (1987), Khan et al., J. Phys. Chem. 89:5180 (1985)), but we postpone such a treatment to a later occasion.

The oil-water-surfactant system in FIG. 19 differs from the two other examples in that the bilayer in the L₃ phase can accommodate the less abundant solvent in addition to the surfactant. Thus the thickness L can vary with concentration. Furthermore for large fractions of solvent in the bilayer, the distance between the two opposing polar/apolar interfaces can show large local variations and the picture of a well-defined base surface breaks down. This complication is particularly pertinent for understanding how the L₃ phase joins with the main microemulsion channel in FIG. 19. However the behavior at low and high α-values is interesting enough. In FIG. 24 we have reproduced the branch of the L₃ phase at low α-values, where water is the abundant solvent. At T=73 C. this branch hits the α=axis and joins with the L₃ phase of the binary surfactant-water system of FIG. 17. Also shown in FIG. 24 is a theoretical line giving the fit of the ternary L₃ region to the present theory. We now describe the derivation of this theoretical curve.

To begin with, we have used the same expression as in the binary case (eq. ((11)) to account for the volume fraction 1−φⱼ of water in the polar region of the surfactant bilayer. The formulae of Cantor do not, however, apply in the case of the less abundant solvent, oil (tetradecane in FIG. 24), because an excess of the solvent was assumed in that theory. In fact, along the curve of interest in FIG. 24 the concentration of oil in the apolar region of the bilayer, β, will be take to be a function only of α. The temperature-dependence of β has been assumed to be negligible, in contrast to the case of water which is present in sufficient quantity to saturate the interface to the concentration given by a the Cantor theory, this latter concentration being a strong function of temperature.

Specifically, we have taken β to be given by:

β=βₘₐₓ/(1+βₘₐₓ),

where

βₘₐₓ=φ=ₒᵢₗ/(φₒᵢₗ+φHC) (12),

φHC representing the volume fraction in the sample due to the hydrocarbon portion of the surfactant; βₘₐₓ thus gives the volume fraction of oil in the apolar portion of the bilayer if all of the oil were located between the surfactant tails. Eq. (12) is the simplest possible formula which at very low oil content puts nearly all of the oil in the interface, and at higher oil contents puts increasing amounts in a separate bilayer between the ends of the surfactant tails. Given the temperature and concentrations, the values of φⱼ and β are computed from equations (11) and (12), and by applying geometrical arguments analogous to those used in the derivation of eq. (8) we arrive at an expression for the area-averaged mean curvature:

<H_{L}>=4γ²/2.2L_{O}(1+φⱼ−β)²=−0.000146/(1+φⱼ−β)²(Å−¹) (13),

where in the last term we have inserted the value β=0.166 for the surfactant concentration in FIG. 24, as well as the estimated value L₀=36 Å for the length of the C₁₂E₅ molecule; for large values of α the half-width L of the bilayer will be larger than this L₄, and this has been incorporated in eq. (13). All that is necessary now to complete the set of equations is an expression for the spontaneous mean curvature H₀.

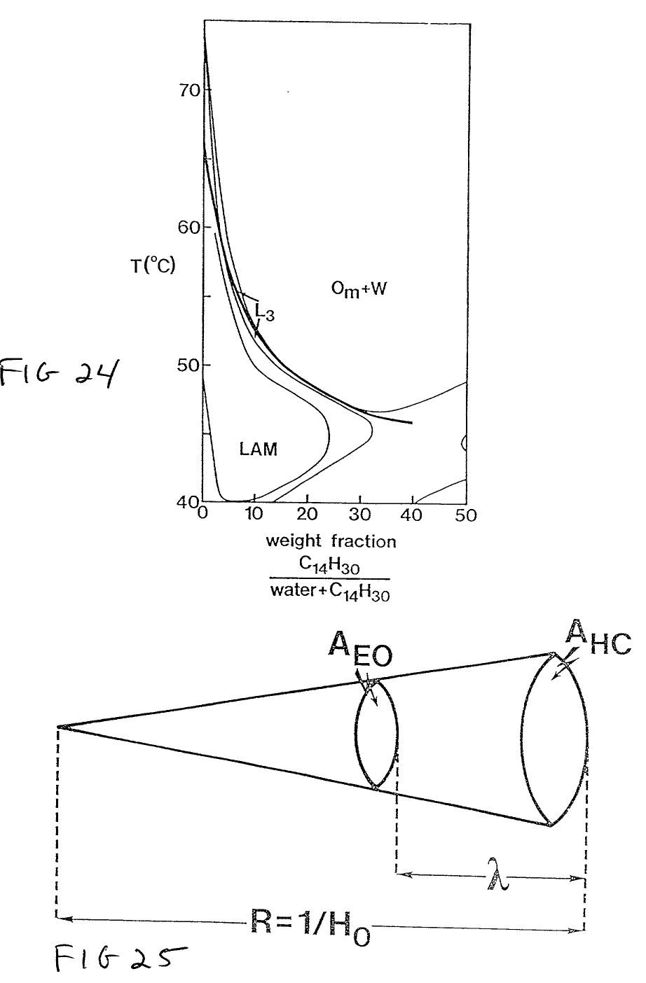

In the present theory, the changes in H₀ are brought about by the penetration of water and oil into the head and tail regions of the bilayer, thus increasing the effective areas A_{EO} and A_{HC} per ethylene oxide and hydrocarbon chain, respectively; a significantly larger effective area A_{HC} on the hydrocarbon side will lead to a significant mean curvature H₀ toward the water. In FIG. 25 we show schematically the relation between the areas A_{EO} and A_{HC}, drawn as spherical caps, and the spontaneous radius of curvature R₀=1/H₀. The distance 4×4 between these hypothetical caps is not entirely unambiguous, but clearly it is between one-half the total surfactant length and the full length. In the present case where we have taken the value of the surfactant length to be L₀=36 Å, we have taken =30 Å Let the superscript (0) refer to the areas A_{EO} and A_{HC} in the absence of solvents. Clearly

$${( {R}_{0} / {R}_{0} - λ ) )}^{2} = {A}_{HC} / {A}_{EQ} \begin{matrix} = ( {A}_{HC} ( 0 ) / ( 1 - β ) ) / {A}^{EO} ( 0 ) / φ J ) \\ = Ωφ j / ( 1 - β )\end{matrix}$$

where Ω=A_{HC}(0)/A_{EC}(0). Thus, solving for H₀=R₀⁻¹ gives

−H₀=(1−\sqrt{[}(1−β)/ΩφJ])/λ

The value of Ω is determined by the condition that, in the binary system (α=β=0), H₀=0 at T=64. C., where φⱼ=0.35 (this value of φⱼ corresponds to approximately 4.5 water molecules per EO group).

This then closes the set of equations, when the conditions <H_{L}>=H_{O}, which expresses the working hypothesis of the papers, is enforced. A computer was used to solve iteratively, at each temperature T of interest, for the value of α at which equations (13) and (15) yield the same value. As can if be seen from FIG. 24, the agreement between theory and data is quite good, especially in view of the fact that no attempt was made to improve the quality of the fit by choosing a form of the relation for β (equation (12)) which contained adjustable parameters. In fact, since the same formula used in the binary case for φ (equation (11), with c′=1) was used in the ternary case, the only adjustable parameter in FIG. 24 is λ, and the results are not sensitive to the value used; since as noted above 18 Å<λ<36 Å is required, we chose λ=30 Å.

Finally we note that there is an analogous behaviour of the L₃ phase at high α-values where oil is the abundant solvent. Also in this case it is necessary to invoke and α-dependence in H₀ to account for the experimentally observed local of the L₃ phase within the model. At low water contents the EO groups overlap and this could lead to an increased tendency to curve towards the oil which in this ease is the most abundant medium. One can note that the stability range of the lamellar liquid crystalline phase is consistent with this conclusion, in that at low α the lamellar phase extends to high temperatures, while at high α it extends to low temperatures (see FIG. 19).

5. Relative Stability of Lamellar, Cubic and L₃ Phases

The L₃ phase occurs in a phase diagram as an alternative to a lamellar phase and it is important to recognize the factors that influence the relative stability of the two phases. In previous studies (Nilsson et al., J. Phys. Chem. 88:4764 (1984); Miller et al., Langmuir 2:321 (1986); and Cates et al., Europhys. Lett. 5:73 (1988)) it has been emphasized that the L₃ phase is a disordered lamellar phase, with the implication that the essential factor favouring the formation of an L₃ phase is entropy. Here we have concluded that the most important factor is the formation of a bilayer structure with the optimal curvature towards the solvent. Clearly lamellar phases are stable over regions much larger than where we can expect that H₀=0 for the constituent monolayer. The curvature energy is thus not the only important contribution to the free energy. There is a free energy cost in forming a continuous bilayer structure in three dimensions in that one introduces local inhomogeneities; as noted in the Appendix, except for a plane, no minimal surface can have constant Gaussian curvature, which would be required in order that H_{L} be constant. At least with a single component in the bilayer it is intrinsically more favourable to have the locally uniform conditions of a planar bilayer rather than locally non-uniform conditions in the L₃ phase. The non-uniform conditions in the L₃ relative to the lamellar phase also affect the free energy contributions from the interbilayer interactions. Also in this case the situation in the lamellar phase with a given interbilayer distance is favourable. In fact is seems to be a necessary condition for the formation of an L₃ phase that the interbilayer interactions are weak. In relation to the lamellar phase this is not so much as to favour disorder, which it does, but rather that strong constraints on interbilayer distances which would favour the lamellar phase are absent.

Another alternative to the L₃ phase can in fact be seen as arising from a melted or disordered cubic structure. In a cubic phase it is also possible to achieve <H_{L}>=H₀ under the same mathematical conditions derived here, and the curvature energy can be at least as favourable in a cubic as in the L₃ phase. Here it is necessary to invoke an important free energy contribution from the disorder present in the L₃ phase. This disordering is favoured by weak interbilayer forces and in FIG. 18 it is seen how the L₃ phase joins up with the cubic phase at high surfactant concentration and thus strong interactions. A similar observation was made in ref. 4 for the nonionic surfactant C₁₆EO₄. In passing we also note that the arguments given for the narrow character of the L₃ phase can also be applied to some cubic phases.

6. Conclusions

It has been concluded that an L₃ phase forms under the condition that the surfactant has locally a bilayer structure. The monolayer has a spontaneous mean curvature H₀ towards the solvent. The average mean curvature of the monolayer <H_{L}> is optimally H₀ and this is realized by the bilayer forming a multiply-connected surface extending in three dimensions. The structure is disordered and undoubtedly undergoing continual thermal disruption. When the interbilayer interactions are weak, the entropy associated with fluctuations of the structure can favour this disordered structure over the ordered cubic phase. However, in contrast with previous work (Cates et al., Europhys. Lett. 5:73 (1988)), we argue that the competition between the L₃ phase and the lamellar phase is not one of entropy differences, but rather mean curvature differences, the L₃ satisfying the negative spontaneous mean curvature H₀ very closely; again in this competition it is necessary that interbilayer interactions be weak, otherwise the lamellar phase will be favoured. Because optimal mean curvature is the main impetus for the formation of the L₃ phase, we expect that it appears only when the condition <H_{L}>=H₀ is very closely satisfied, and we have shown that for a given H₀ the volume fraction is then uniquely given, thus rationalizing the narrowness of the L₃ phase regions.

In order to minimize the curvature energy, we have used minimal surfaces as models for the base surface, but we have refrained from giving a detailed picture of the structure in the L₃ phase. It has been possible to arrive at the general thermodynamic consequences without a detailed structural model, particularly in view of the apparent constancy of the ratio XE^{u}/Σ³, which is where the properties of the model base surface enter. Furthermore few attempts have been devoted to scattering or spectroscopic studies of the L₃ phase, partly because of the experimental difficulties of preparing a one phase sample. The striking diffusion results, that have been taken as a strong piece of evidence in favour of a lamellar structure are in fact equally consistent with a cubic structure (Anderson et al, To be published) and then most likely also with disordered structures with the same basic units. In particular, it has been proven by analytical calculation that the effective self-diffusion coefficient for a particle (viz., a surfactant head group) diffusing over an arbitrary minimal surface of cubic symmetry is exactly the same as that of the same particle diffusing in a lamellar structure, namely (Anderson et al, To be published) the obstruction factor is ⅔.

We have argued that the narrowness of the L₃ phase region is due to a constraint on the area-averaged mean curvature <H_{L}> of the polar/apolar interface, so that deviations of <H_{L}> from the spontaneous mean curvature H₀ are too costly, in view of the small free energy differences between the competing microstructures. This is the reason why it is particularly important that, at least in the limiting case of triply-periodic order, the results derived above using the parallel-surfaces description of the interface also hold for the closely-related surfaces of constant mean curvature, as was shown above. In the parallel-surface description, there is considerable variation in H_{L} over the interface, so that even though <H_{L}>=H₀ there are large deviations from H₀ pointwise. However, this is simply a consequence of the high sensitivity of H₀, which is a second derivative property, to the exact shape of the interface. Analysis of newly-discovered periodic surfaces of constant mean curvature (Anderson, D., Ph.D. Thesis, University of Minnesota (1986)) shows that, by allowing variations in the bilayer width on the order of 7%, the condition that H_{L}=H₀ can be satisfied pointwise over the entire interface (Anderson et al., Proc. Natl. Acad. USA, In press (1988)), at least for periodic structures. Because the study of these constant-mean-curvature surfaces is in its infancy, and because the traditional approach to the study of monolayer and bilayer shapes has been in terms of the curvature energy, we have used the parallel-surface description for most of the derivations. However, as argued elsewhere (Anderson et al., Proc. Natl. Acad. USA, In press (1988)), the constant-mean-curvature description appears to provide a more realistic description of the local inhomogeneities, and in analogy with the results given in ref.33 we argue that the bilayer in the L₃ phase can be fairly homogeneous in both width and mean curvature.

During the completion of this work we became aware of a recent small angle neutron scattering and conductivity study of some dilute surfactant/alcohol/brine systems by Porte et al. (Porte, et al., J. Phys. (France) 49:511 (1988)). For the L₃ phase, termed L₂^{*} by the authors, they conclude that the structure is locally a bilayer, from an analysis of the position of a broad hump in the scattering curves as a function of water concentration. They then address the matter of the larger-scale, topological description of the structure. Clearly a ‘foam’ structure, which has the same topology as an inverse micellar phase, is difficult to reconcile with the high conductivities. Certain other model structures are evaluated on the basis of a quantitative analysis of the position q_{c} of the hump in the scattering curves, in which it is assumed that the distance d^{*}=2π/q_{c} can be taken as an estimated cube size in a cubic tesselation with the bilayer lying on some of the cube faces. However, in such a picture the relation between q_{c} and the lattice parameter is not necessarily as simple as d^{*}=2/q_{c}, because for example the case illustrated in their FIG. 13 is of BCC symmetry (space group Im3m), so that the first scattering peak would occur at q_{c}=√2×2/d^{*}. In fact, recent work by Siegel₅₃, and by S. Leibler and T. Maggs (personal communication) has shown that the distinction between the bicontinuous topology and the lamellar phase with a high density of defects (ILA's) may be tenuous. With these cautionary comments in mind, the results of Porte et al. are consistent with the present model, as is the position of the L₂* phase relative to the Lα in their study: the L₂* lies at higher hexanol concentrations, and an average mean curvature toward water at these compositions is thus consistent with a reversal in spontaneous mean curvature from toward the apolar regions in the L₁ phase at low hexanol, to zero mean curvature in the L_{α} phase at intermediate hexanol concentrations, to towards water in the L₂* at higher hexanol concentrations.

The discussion in this paper has been basically confined to ‘typical’ L₃ phases. Since this is an isotropic solution it can continuously join with other isotropic solution. FIG. 19 shows how the L₃ branch is connected by a two-phase region to the isotropic L₂ phase at high surfactant concentrations. A detailed discussion of the structural changes occurring in the transition from one ‘type’ of phase to another should await further experimental studies of the systems. There exist also a number of systems where isotropic solution phases in some region show the narrow character that is typical of the L₃ phase, as for example in L₂ region of the H₂O-sodium octanoate-octanoic acid system⁴⁵; this is in fact closer to the behaviour in the previously mentioned system from reference 15 involving a zwitterionic surfactant, as well as that in the C₁₂E₃/water L₂ phase. At present we cannot determine whether or not there are any fundamental differences between those systems in which the L₃ phase region is disconnected from the L₂ (or joined by a two-phase L₂/L₃ coexistence region), and those systems in which the L₂ is more tenuous, particularly in ternary systems such as that in FIG. 19, in which the L₃ phase connects continuously (apparently) to the main microemulsion channel, where the latter channel progresses continuously from normal micellar solutions to inverted micellar solutions. We mention also the possibility that the L₃ phases in the binary ethoxylated alcohol systems may be essentially structureless solutions, in which case the L₂/L₃ coexistence would represent coexistence between a microstructured (L₃) and a structureless (L₂) solution.

As a final comment we note that the L₃ phase has a biologically highly interesting counterpart in the membrane system of the endoplasmatic reticulum (ER). Similar structures have apparently also been seen by Helfrich and Harbich in pure phospholipid-water systems (Helfrich et al., in “Physics of Amphiphilic Layers” eds. J. Meunier et al., Springer Verlag Berlin, p.58 (1987)).

### Appendix

In this invention it is proven that if a bilayer of constant width 2L is a local minimum of the curvature free energy G_{c} (equation 4), then the base surface representing the midplane of the bilayer must be a minimal surface. We stress that this is only a necessary condition, and not in general sufficient. The question of whether or not a bilayer structure based on a given minimal surface is in fact stable to local or global perturbations is much more involved, and although the present proof will show that only minimal surfaces need be considered as possible solutions to this stability question, we defer a full discussion of this question to a later date. It is noted that the present results remain valid even in the case where a saddle splay term (Helfrich, W. Z., Naturforsch. 28c:693 (1973)), proportional to the integral Gaussian curvature, is included.

In this Appendix an elementary proof is given which does not require the usual complex variable approach to the theory of minimal surfaces and constant mean curvature surfaces, of the fact that except for the case of planes (lamellae), a bilayer of constant width cannot also have constant mean curvature. Thus, as stated in the text, for the case of nonzero spontaneous mean curvature H₀, inhomogeneities in the bilayer are unavoidable.

Although the present application of this calculation is to the L₃ phase, it should be mentioned that the same results apply to binary surfactant/water cubic phases, and it is important to note that in all of the structures which have been substantiated for the cubic phases, with one exception, a minimal surface has been found which describes the midplane of the bilayer (see ref. 41 for a review). The exception is the discrete cubic phase of space group Pm3n, composed of elongated micelles (Fontell et al., Mol.Cryst.Liq.Cryst. 1(1-2):9 (1985)), where mean curvature energies appear to be a relatively minor factor in determining the structure.

In singling out curvature energies as the sole energy contribution in this calculation, we are of course exploring the consequences of only one limiting case, and in particular by ignoring entropic effects we are doomed to periodic solutions for the solution to the more specific problem, not treated here, of determining those structures that are in fact stable with respect to arbitrary perturbations. However, we are not seeking actual stable solutions here but rather deriving one property which is required of a local minimum, namely that the base surface is of zero mean curvature, and with this it can be argued that the base surface in the periodic L₃ phase is tending toward zero mean curvature in order to minimize the curvature free energy, throughout the course of thermally-driven fluctuations. Presently work is in progress to compute periodic surfaces of exactly zero mean curvature (Bohlen, D. Ph.D., Univ. of Minnesota; work in progress), which should be instructive. Before proceeding with their derivation, we again point out that there is an alternative description of the bilayer shape in terms of constant mean curvature surfaces. Triply-periodic surfaces of constant mean curvature have recently been discovered (Anderson, D., Ph.D. Thesis, University of Minnesota (1986); and Karcher, H. Preprint, Bonn, (1987)), and certain of these surfaces can be used to describe continuous-bilayer structures, which are symmetric with respect to a base surface that is minimal surface (Hyde et al., Z. Kristallogr. 168:213 (1984); and Anderson et al., Proc. Natl. Acad. USA, In press (1988)). In such a description the curvature energy given above can be made to vaqnish, but one can assign an energy cost to variations in the bilayer width—a stretching energy. One could then investigate a statement analogous to that treated in this Appendix, namely: if a bilayer with constant mean curvature at the polar/apolar interface is a local minimum of the stretching energy, then the midplane of this bilayer must be a minimal surface. However, to date the knowledge of surfaces of constant, nonzero mean curvature is too limited to permit any such analysis.

Special consideration of the class of perturbation in the present analysis is given because this class will be sufficient to prove that the base surface minimizing the curvature energy must necessarily be a minimal surface. This class will be the class of so-called ‘inextensional’ perturbations (Weatherburn, C. “Differential Geometry of Three Dimensions”, 2 vols., Cambridge University Press (1926). An inextensional deformation is one in which the length of any element of arc on the surface remains unchanged. Thus the coefficients of the first fundamental form remain unchanged, and by Gauss' Theorema Egregium, the Gaussian curvature remains unchanged. Furthermore, the differential area element dA remains unchanged. However, the mean curvature can change.

For an arbitrary base surface S, with mean curvature H(u,v) and Gaussian curvature K(u,v), the curvature energy G_{c} over the two displaced (parallel) surfaces a constant distance L away from S is given by:

$$\begin{matrix}{G}_{C} = {K}_{B} \{ \int {\int }_{S} [ \frac{H + LK}{1 + 2 LK + {L}^{2} K} - {H}_{0} ] 2 ( 1 + 2 LK + {L}^{2} K ) ⅆ A + \\ \int {\int }_{S} {[ \frac{- H + LK}{1 - 2 LK + {L}^{2} K} - {H}_{0} ]}^{2} ( 1 - 2 L H + {L}^{2} K ) ⅆ A \}\end{matrix}$$

using the well-known formula for the mean curvature of a parallel surface in terms of the mean and Gaussian curvatures of the base surface. We wish to test a base surface S_{b} for stability with respect to inextensional perturbations. Such a perturbation of S_{b} changes only the mean curvature H_{b} in equation (A1), to a new point function which we will call H_{ε}, where:

H_{ε}(u,v)=H_{b} (u,v)+εQ(u,v)

Q being an arbitrary test function., The Euler equation to be solved is thus:

$$\frac{ⅆ}{ⅆ ϵ} \vert ϵ = 0 \qquad {G}_{C} [ {H}_{ϵ} ] = 0$$

This becomes, upon simplification:

$$4 {K}_{b} \int {\int }_{{S}_{b}} \frac{{{H}_{b} ( 1 - {L}^{2} K )}^{2} ( 1 + {L}^{2} K )}{{( 1 + 2 L {H}_{b} + {L}^{2} K )}^{2} {( 1 - 2 L {H}_{b} + {L}^{2} K )}^{2}} Q ⅆ A = 0$$

In order for this to vanish for all test functions Q, it is necessary that either:

H_{b}(u,v)0

K(u,v)=±1/L²

for all (u,v). The first condition (A4) expresses the fact that S_{b} is a minimal surface. We show below that the second condition (A5) is unphysical.

Before proceeding to this, however, we note that these same conditions result from a much simpler requirement, namely that the value of the mean curvature at the two points, one on each displaced surface, which correspond to the same point on the base surface (i.e., with the same surface coordinates (u,v)), be the same, for each point on the base surface. Write H_{L}+ and H_{L}− for these two mean curvature values, and:

$${H}_{L}^{+} - {H}_{L}^{-} \frac{{H}_{b} + L K}{1 + 2 L {H}_{b} + {L}^{2} K} - \frac{- {H}_{b} + L K}{1 - 2 L {H}_{b} + {L}^{2} K} = \frac{2 {H}_{b} ( 1 - {L}^{2} K )}{( 1 + 2 L {H}_{b} + {L}^{2} K ) ( 1 - 2}$$

The condition that this difference vanish is given by (A4) or (A5). This can be expressed by saying that when, and only when, the base surface is a minimal surface, the bilayer has an additional symmetry with respect to the mean curvature of the two displaced surfaces.

We now show that the condition (A5) is unphysical, although interesting in the light of Bonnet's theorem. Bonnet's theorem states that the surface at a constant distance L from a surface of constant Gaussian curvature equal to −1/L² is of constant mean curvature. This is interesting in that if this situation were physical realizable, then we would be lead to interfaces of constant mean curvature (‘Bonnet translates’), as well as of constant width; in such a world one might expect to find base surfaces with constant Gaussian curvature. However, in deriving these results we are assuming that the polar/apolar interface lies at a constant distance L along the normal to the base surface. And in the case where the Gaussian curvature of the base surface is of magnitude 1/L₂, these normals, representing surfactant molecules, will necessarily intersect. This is because the Gaussian curvature K=k₁k₂ is of magnitude 1/L², then one of the principal curvatures, say k₁, must be of magnitude greater than or equal to 1/L. Rays of length L drawn from points along this line of curvature along the normal direction must intersect. This can also be seen by noticing that when L=1/┘k₁}, then the quantity 1-2LH+L²K vanishes, so that the differential area element dA_{L} vanishes, and the mean curvature H_{L} diverges—both signifying that the normal rays have intersected. Thus, the solution given by equation (A5) is physically unrealizable under the present assumptions, although in view of the fact that the Euler equation (A4) was derived without first constraining the problem to rule out unphysical solutions, it was necessary that (A5) be found as a formal solution, at least in the case where the mean curvature, −1/L, of the Bonnet translate equals the spontaneous mean curvature.

It was stated in the main text that inhomogeneities in the bilayer are a necessary consequence of nonzero spontaneous mean curvature. We have now shown that the requirements of homogeneity in width and in mean curvature (using equation (A6) lead to the necessary condition that the base surface S_{b} be a minimal surface. We now show that this condition is never in fact sufficient, except in the case of H₀=0 (lamellae); that is, in the case of nonzero spontaneous mean curvature H₀, the mean curvature over the polar/apolar interface cannot be identically H₀ when the width is constant. In ref. 33 this was referred to as ‘frustration’. We now give an elementary proof of this, based on a formula from elementary differential geometry known as the Mainardi-Codazzi relation, which singles out the basic cause for this frustration, in a way that is more intuitive, perhaps, than the usual proofs using the theory of complex variables in the treatment of minimal surfaces. Furthermore, this formula (equation (A9) below) will be important in an in-depth analysis of the more general stability problem, which will be the subject of a future publication, and we give here a simpler instance of its importance. The Mainardi-Codazzi relation is also pivotal in the (rather involved) proof, due to Hilbert, that there exists no complete surface with constant, negative Gaussian curvature (Willmore, T. “An Introduction to Differential Geometry”, Oxford University Press, London (1959)).

The base surface S_{b} must be a minimal surface, H_{b}=0. The mean curvature over the polar/apolar interfaces then given by:

H_{L}=LK₀/(1=L²K₀)

In order for H_{L} to be constant, it is clear that the Gaussian curvature over the base surface K₀ must be constant, and that this constant value be nonzero if we require H_{L}=H₀. At this Point the usual complex variable approach⁵² can be used to show that the Gaussian curvature of a minimal surface cannot be constant, but we use instead a formula derived from the Mainardi-Codazzi relation, a fundamental relation in the differential geometry of arbitrary surfaces.

We take the (u,v) parametric curves to be the lines of curvature with k corresponding to the direction v-const., and then −k is the curvature along the direction u=const. The Mainardi-Codazzi relations are then:

(1/\sqrt{E})dK/du=−K(dG/du)/G\sqrt{E}=−2KKgu,

(1/\sqrt{G})dK/dv=−K(dE/dv)/E\sqrt{G}=2KKgv

using the usual formula for the geodesic curvatures k_{gu} and k_{gv} of the lines u=const. and v=const., resp. But the left hand sides of these equations represent the two components of the surface gradient of k. In the more general case of a surface of constant mean curvature, and for the present case of a minimal surface, this can be expressed in the alternative form (Willmore, T. “An Introduction to Differential Geometry”, Oxford University Press, London (1959)).

∇_{S}K=−(K1−K2)²(aKgv+bKgu) (H=constant),

∇_{S}K=4K(aKgv+bKgu) (H=0)

where ∇ₛ is the surface operator and a and b are the unit vectors in the u and v directions. This second equation (A9) is the heart of the present argument, because it is straightforward to show that the geodesic curvatures of the lines of curvature cannot both vanish identically on a minimal surface except when K=0, so that by (A8) (or (A9)), the gradient of K cannot vanish, except for the case of the plane.

To prove this, assume that k_{gu}=k_{gv}=0 at every point of S_{b}. Then we apply Liouville's formula, which states that the geodesic curvature, along a line which makes an angle ν with the curve v=const., is k_{g}=dv/ds+k_{gv} cos v+k_{gv} sin ν. In particular, consider the line given by ν=π/4; by this formula K_{g}=0 along such a curve, and by Euler's theorem for the normal curvature kₙ=k₁ cos²ν+K_{g} sin²ν=0, using k₂=−k₁. But then the space curvature k=√(k_{g}²+kₙ²)=0, and this means that the surface is a ruled surface because there is a straight line through every point. However, as is well known, the only minimal surface that is also a ruled surface is the right helicoid, which can be verified by solving a simple o.d.e. for the vanishing mean curvature of a ruled surface (analogous to the proof that the catenoid is the only minimal surface of revolution). Since the right helicoid is not of constant Gaussian curvature (the steps taken above are necessary but not sufficient), the proof is finished.

What is claimed:

1. A stabilized microporous material comprising:
a continuous, regular, branched and interconnected porespace morphology comprising pore bodies and pore throats, having a globally uniformm effective pore size, in which the pore bodies and the pore throats are substantially identical in size and shape respectively,
wherein the stabilized material arises from polymerization of an unpolymerized precursor component in combination with other components, all components forming a bicontinuous cubic phase at thermodynamic equilibrium, in a process comprising the steps of:
a. combining said unpolymerized precursor component with said other components,
b. thoroughly mixing all components,
c. allowing the resulting mixture to equilibrate to the bicontinuous cubic phase, and
d. polymerizing said precursor component.

2. A material as recited in claim 1, wherein the unpolymerized precursor component is in an aqueous phase.

3. A material as recited in claim 1, wherein the unpolymerized precursor component is in an hydrophobic phase.

4. A material as recited in claim 1 wherein the unpolymerized precursor component is in a polymerizable surfactant phase.

5. A stabilized microporous material as recited in claim 1 and having an effective pore diameter on the order of 10 nanometers.

6. A stabilized microporous material as recited in claim 1, wherein the porespace is isotopic and triply-periodic, and
wherein the effective pore size is larger than 2 nanometers (20 Angstroms).

7. A stabilized microporous material as recited in claim 1, wherein the standard deviation of effective pore size is of the order of magnitude of 3%, and
wherein the effective pore size is larger than 2 nanometers (20 Angstroms).

8. A material as recited in claim 1, wherein biologically active agents are incorporated at substantially preselected locations in the porespace.

9. A material as recited in claim 1, wherein the poresepace is at least partially filled with an active fluid agent for actively or passively controlled release.

10. A material as recited in claim 1, wherein said material consists essentially of biocompatible materials.

11. A polymeric microporous material comprising two distinct, interwoven but mutually disconnected porespace labyrinths, each of which is continuous, regular, branched and interconnected with itself, each having globally uniform effective pore size; the distinct pore space labyrinths separated by a continuous stabilized dividing wall, the wall having two distinct surfaces, each surface facing one respective porespace labyrinth.

12. A polymeric microporous material as recited in claim 11, wherein the two district surfaces have different ion selectivity.

13. A polymeric microporous material as recited in claim 11, wherein the two distinct surfaces have different chiralty characteristics.
